(19) United States
(12) Patent Application Publication
(10) Pub. No.: US 2019/0277833 A1
(43) Pub. Date: Sep. 12, 2019

(54) ANTI-METHANOGENIC COMPOSITIONS

(71) Applicant: Synthetic Biologics, Inc., Rockville, MD (US)

(72) Inventors: Klaus GOTTLIEB, Rockville, MD (US); Michael KALEKO, Rockville, MD (US); Vincent J. WACHER, Rockville, MD (US); Steven M. MUSKAL, Rockville, MD (US)

(21) Appl. No.: 16/302,425

(22) PCT Filed: May 12, 2017
(86) PCT No.: PCT/US2017/032454

§ 371 Date: Nov. 16, 2018

Related U.S. Application Data
(60) Provisional application No. 62/338,549, filed on May 19, 2016.

(51) Int. Cl. G01N 33/50; A61P 3/10; A61K 31/45; A61K 31/366; A61K 31/40; A61K 31/4192; A61K 31/4375; C12N 9/06; C12N 9/02; A61P 1/06; A61P 3/04

(52) U.S. Cl. CPC G01N 33/5041; A61P 3/10; A61K 31/45; A61K 31/366; A61K 31/40; A61K 31/4192; A61K 31/551; C12N 9/0026; C12N 9/0095; C12N 9/0051; A61P 1/06; A61P 3/04; A61K 31/4375

(57) ABSTRACT

The present invention relates, in part, to treatment of various diseases and disorders such as GI disorders through modulation of F420-dependent enzymes including specific enzymes involved in methanogenesis.

### CROSS-REFERENCE TO RELATED APPLICATION

This application claims priority to U.S. Provisional Application No. 62/338,549, filed May 19, 2016. The contents of which is incorporated herein by reference in it's entirely.

### FIELD OF THE INVENTION

The present invention relates, in part, to treatment of various diseases and disorders such as GI disorders using compounds that modulate F₄₂₀-dependent enzymes.

### BACKGROUND OF THE INVENTION

The microbiome, which refers to the community of commensal, symbiotic, and pathogenic microorganisms living in humans and other animals, plays an important role in both health and disease. For example, while the majority of microorganisms inhabiting the gastrointestinal (GI) system of humans have a beneficiary role in, for example, aiding digestion, a minority of such commensal organisms have been implicated in the pathogenesis of various diseases.

Recent studies have suggested that certain methane producing microorganisms inhabiting the gut known as methanogens may play a causative role in a number of GI diseases and disorders. Methane (CH₄) production in humans is derived from methanogenic archaea in the intestines. These organisms serve a critical biological function by removing the by-products of bacterial fermentation of polysaccharides, notably hydrogen gas (H₂) and short-chain fatty acids (SCFAs). The dominant methanogen inhabiting the human gut is the archaea, Methanobrevibacter smithii (M. smithii). In vitro susceptibility testing has demonstrated that methanogens such as M. smithii are highly resistant to most classes of antibiotics. Further, complete eradication of intestinal methanogens via a single course of therapy is unlikely using broad spectrum antibiotics, leading to methanogen recolonization and methanogenesis returning to pathogenic levels. Continuous use of antibiotics is also associated with various side effects and increased risk of developing antibiotic resistance. Further still, long-term use of antibiotics may disrupt the otherwise potentially beneficial bacterial intestinal microbiome and gastrointestinal flora.

Statins are among the most commonly prescribed drugs in the world. Statins are a class of cholesterol-lowering drugs that inhibit the enzyme HMG-CoA reductase (HMGR), an enzyme that plays a central role in the production of cholesterol. Recent studies have indicated that statins can also impact methanogenesis. Importantly, statins have their methane-reducing effects in a microbiome-protecting manner and exert their activity by a targeted effect on methane-producing organisms in a non-microbicidal manner thus avoiding collateral damage to the gut microbiome (and therefore are distinguishable from antibiotics, for example). Specifically, statins appear to inhibit archaeal growth. Consistent with the classical HMGR-inhibitory mode of action, it is generally believed that statins inhibit methane production via their effect on cell membrane biosynthesis, mediated by inhibition of HMGR. Specifically, HMGR catalyzes the first step in the biosynthesis of the archaeal isoprene polymers (polyprenols) archaeol and caldarchaeol, which are the main constituents of the M. smithii cell membrane.

However, the HMGR/statin interaction assumes that statins are present in the open ring hydroxyacid form. This is the bioactive form used for cholesterol-lowering pharmacologically. But, recent data has shown that the closed-ring lactone form of statins is particularly useful for methane reduction. This suggests that the beneficial microbiome effects of statins may be provided in a HMGR-independent manner.

What is needed is a more complete understanding of the pharmacology of statins for use in modulating methanogenesis, including for use in the treatment of various human GI diseases and disorders. Further still, there remains a need for safe and effective therapeutic agents for the long term suppression of enteric methanogenesis and/or excessive methane production in the treatment of methanogen-related diseases and disorders.

### BRIEF SUMMARY OF THE INVENTION

Accordingly, the present invention provides, inter alia, methods of modulating F₄₂₀-dependent enzymes (i.e., enzymes that utilize F₄₂₀ as a coenzyme) in a subject. In various embodiments, the F₄₂₀-dependent enzymes are methanogenesis-related enzymes such as those enzymes that participate in the methanogenesis pathway. In an embodiment, the F₄₂₀-dependent enzyme is the F₄₂₀-dependent enzyme mtd (UniProt designation—A5UMI1 (F₄₂₀-dependent methylenetetrahydromethanopterin dehydrogenase)).

In one aspect, the present invention provides methods for identifying statin analogs or derivatives and/or compounds that can modulate specific F₄₂₀-dependent enzymes. In some embodiments, methods of the invention allow for the identification of statin analogs or derivatives and/or compounds that have high affinity for methanogenesis-related F₄₂₀-dependent enzymes. In an embodiment, the present invention provides methods for identifying statin analogs or derivatives and/or compounds that have high affinity for mtd/A5UMI1 (F₄₂₀-dependent methylenetetrahydromethanopterin dehydrogenase). In some embodiments, the statin analogs or derivatives and/or compounds bind to and inhibit the activity of the methanogenesis-related F₄₂₀-dependent enzymes.

In another aspect, the methods described herein eradicate or reduce methane production, which is causative of, or correlative with, various methanogen-associated disorders, including, for example, constipation, irritable bowel syndrome (IBS) (e.g. irritable bowel syndrome with constipation (IBS-C)), diabetes and obesity. In an embodiment, the present invention provides for methods of inhibiting or reducing methanogenesis and/or methane accumulation by administering a statin, a statin analog or derivative, or a compound or formulation disclosed herein, inclusive of compounds of Formulas I-VI and Compounds (1)-(131), to a subject in need thereof. In various embodiments, the present invention provides for methods of treating or preventing a methanogen-associated disorder optionally selected from one or more of IBS, such as IBS-C, diabetes, and obesity by administering a statin, a statin analog or derivative, or a compound or formulation disclosed herein, inclusive of compounds of Formulas I-VI and Compounds (1)-(131), to a subject in need thereof. In an embodiment, methods are provided for treating constipation using a statin, a statin analog or derivative, or a compound or formulation described herein. In another embodiment, methods are provided for reducing or eliminating enteric methane production using a statin, a statin analog or derivative, or a compound or formulation described herein.

In various embodiments, the present methods provide for a manner for selecting patients who are likely to respond to treatment with a statin, a statin analog or derivative, and/or a compound disclosed herein such as the lactone form of a statin or statin analog or derivative or a compound disclosed herein. For instance, the patients can be gastrointestinal (GI) disorder patients, such as, for example, irritable bowel syndrome patients. Accordingly, the various methods of treatment described herein may involve a step of profiling a patient for methanogenesis-related F₄₂₀-dependent enzyme status. Various methods are provided that allow for patient evaluation, e.g., diagnosis or prognosis, based on the patient's F₄₂₀-dependent enzyme status. In some embodiments, methods of the invention are directed to patients who are likely to respond to treatment with a lactone form of a statin or a stain analog or derivative based on their F₄₂₀-dependent enzyme status. In some embodiments, methods of the invention are directed to patients who are likely to respond to treatment with a compound disclosed herein based on their F₄₂₀-dependent enzyme status.

In various embodiments, the present invention relates to a pharmaceutical composition comprising one or more compounds of Formulas I-VI and Compounds (1)-(131) and a pharmaceutically acceptable excipient.

### BRIEF DESCRIPTION OF THE SEVERAL VIEWS OF THE DRAWINGS

FIG. 1 shows the methanogenesis pathway and a potential role of lovastatin. Coenzymes F₄₃₀ and F₄₂₀ play crucial roles in the methanogenesis pathway. In-silico molecular docking of the methanogenic enzyme F₄₂₀-dependent NADP oxidoreductase (fno) showed that both lovastatin and mevastatin had higher affinities for the F₄₂₀ binding site on fno than did F₄₂₀ itself. As such, lovastatin acts as inhibitor of fno. Fno is not shown in the diagram because it catalyzes an alternative pathway of methanogenesis that utilizes alcohol and methanol as substrates. Without wishing to be bound by theory, it is believed that other enzymes, such as those in the hydrogen-CO₂-methanogenesis pathway as depicted herein, that require the coenzyme F₄₂₀ would also be bound and/or inhibited by lovastatin. MF—methanofuran, MP—methanopterin, CoM—coenzyme M, Fd—ferredoxin, CoB—coenzyme B.

FIG. 2 shows coenzymes in the methanogenesis pathway. The final step in the methanogenesis pathway is catalyzed by the key enzyme methyl-coenzyme M reductase (Mcr). In its active site Mcr contains a unique active group, a nickel porphinoid (left panel), called coenzyme F₄₃₀. Coenzyme F₄₂₀ (right panel) participates in two earlier steps in the methanogenesis pathway and is also responsible for the characteristic fluorescence of methanogens. In-silico protein-ligand docking experiments suggest that lovastatin may have higher affinity for the F₄₂₀ binding site than F₄₂₀ itself.

FIG. 3 shows that lovastatin lactone may have a different target in archaea than the hydroxyacid form. Simvastatin and lovastatin are commercially available statins that exist in the lactone form. Their cholesterol-lowering effect and the impairment of archaeal membrane synthesis through inhibition of HMGR require activation, i.e., the lactone ring needs to be opened to result in the hydroxyacid form. As can be seen, the stereochemistry of lovastatin lactone and hydroxyacid is significantly different. Without wishing to be bound by theory, it is believed that methanogenesis is preferentially inhibited by the lactone form of lovastatin and that lovastatin may have a different or an additional target other than HMGR. Possible targets for the lactone form are enzymes in the methanogenesis pathway that have F₄₂₀ as coenzyme.

FIG. 4A and FIG. 4B depict some embodiments of a modified-release formulation in the form of encapsulated beads which releases a first dose of statin at the duodenum and a second dose of statin at the ileum. Any of the statin analog or derivative or compound described herein may also be similarly formulated.

FIG. 5 depicts embodiments of modified-release formulations as multi-layer capsules or tablets for delivery of statins to the intestines (an illustrative commercial material is shown, related materials are known in the art). Any of the statin analog or derivative or compound described herein may also be similarly formulated.

FIG. 6A and FIG. 6B depict embodiments of modified-release formulations for colonic delivery of statins (an illustrative commercial material is shown, related materials are known in the art). Any of the statin analog or derivative or compound described herein may also be similarly formulated.

FIG. 7 depicts various embodiments of modified-release formulations in the form of capsules that delivers either one or two doses of statins to the intestines. Any of the statin analog or derivative or compound described herein may also be similarly formulated.

FIG. 8 shows a modeled quaternary structure of A5UMI1/31QZB (cyan) and A5UMI1/3IQZF (pink) after respective alignments onto chain-B and chain-F of 3IQZ within PyMOL.

FIG. 9 shows the top two scoring sites were A5UMI1_3IQZB and Q02394_4JJF.

FIG. 10 shows lovastatin and F420 docked into A5UMI1_3IQZB_SiteSeeker2.

### DETAILED DESCRIPTION OF THE INVENTION

The present invention is based, in part, on the surprising discovery that statins can directly modulate enzymes that utilize F₄₂₀ as a coenzyme (“F₄₂₀-dependent enzymes”). Specifically, statins (e.g., the lactone forms of statins) directly targeted enzymes in the methanogenesis pathway that have F₄₂₀ as a coenzyme (“F₄₂₀-dependent enzymes”), including mtd/A5UMI1 (F₄₂₀-dependent methylenetetrahydromethanopterin dehydrogenase). The present invention further provides compounds that bind to and target methanogenesis-related F₄₂₀-dependent enzymes.

### F₄₂₀-Dependent Enzymes

In various embodiments, the present invention provides methods of modulating the activity of various F₄₂₀-dependent enzymes, i.e., enzymes that uses F₄₂₀ as a coenzyme, using a statin, a statin analog or derivative, and/or other compounds described herein, inclusive of compounds of Formulas I-VI and Compounds (1)-(131). Coenzyme F₄₂₀ or 8-hydroxy-5-deazaflavin is a coenzyme involved in redox reactions in many methanogens and other bacterial lineages. The F₄₂₀ coenzyme is a flavin derivative.

Accordingly, in various embodiments, the present invention provides methods of modulating the activity of methanogenesis-related enzymes that use F₄₂₀ as a coenzyme. For example, the statin, statin analog or derivative, and compound disclosed herein may target an enzyme in the methanogenesis pathway, such as, for example, one or more of adh alcohol dehydrogenase, fdh formate dehydrogenase, mtd/A5UMI1 (F₄₂₀-dependent methylenetetrahydromethanopterin dehydrogenase), fno F₄₂₀-dependent NADP oxidoreductase, ftr formyl-MF:H4MPT formyltransferase, fwd formyl-MF dehydrogenase, hmd methylene-H4MPT dehydrogenase, mch methenyl-H4MPT cyclohydrolase, mtd F₄₂₀-dependent methylene-H4MPT dehydrogenase, mer F₄₂₀-dependent methylene-H4MPT reductase, mtr methyl-H4MPT:CoM-methyltransferase, mcr methyl-CoM reductase, mtaB methanol:cobalamin methyltransferase, and the heterodisulfide reductase system.

In some embodiments, the statin, statin analog or derivative, or compound disclosed herein directly targets methanogenesis-related enzymes that utilize F₄₂₀ as a coenzyme and/or as a substrate. Exemplary methanogenesis-related F₄₂₀-dependent enzymes include, but are not limited to, mtd/A5UMI1 (F₄₂₀-dependent methylenetetrahydromethanopterin dehydrogenase), fno F₄₂₀-dependent NADP oxidoreductase, mtd F₄₂₀-dependent methylene-H4MPT dehydrogenase, mer F₄₂₀-dependent methylene-H4MPT reductase, coenzyme F₄₂₀ hydrogenase, methylenetetrahydromethanopterin dehydrogenase, and F₄₂₀-dependent sulfite reductase. In an embodiment, the statin, statin analog or derivative, and compound disclosed herein directly targets mtd/A5UMI1 (F₄₂₀-dependent methylenetetrahydromethanopterin dehydrogenase). In various embodiments, the mtd/A5UMI1 is derived from Methanobrevibacter smithii or Methanobrevibacter ruminantium.

In some embodiments, the statin, statin analog or derivative, or compound disclosed herein inhibits the activity of the methanogenesis-related enzymes. In an embodiment, the statin, statin analog or derivative, or compound disclosed herein inhibits the activity of mtd/A5UMI1 (F₄₂₀-dependent methylenetetrahydromethanopterin dehydrogenase). In various embodiments, the activity of the methanogenesis-related enzymes is inhibited resulting in reduced methanogenesis.

### Statins

As used herein, “statin” refers to a class of compounds that is known in the art as inhibitors of HMG-CoA reductase used as lipid lowering agents. However, the prior use of the statin compounds does not necessarily imply a mechanism of action in the treatment of other diseases or disorders such as, for example, methanogenesis. That is, in some embodiments, the statin may inhibit the enzyme HMG-CoA reductase while in others it may have another manner of causing an effect. In some embodiments, the statin does not substantially inhibit the enzyme HMG-CoA reductase.

Illustrative statins useful for the invention include, but are not limited to, atorvastatin, cerivastatin, dalvastatin, eptastatin, fluindostatin, fluvastatin, lovastatin, mevastatin, pitavastatin, pravastatin, rosuvastatin, simvastatin, velostatin, and pharmaceutically acceptable esters, prodrugs, salts, solvates, enantiomers, stereoisomers, active metabolites, co-crystals, and other physiologically functional derivatives thereof. In one embodiment, the statin is lovastatin. In another embodiment, the statin is mevastatin. In yet another embodiment, the statin is simvastatin. In some embodiments, the statin is in either the lactone or hydroxyacid form. In some embodiments, the statin is the lactone form of one or more of atorvastatin, cerivastatin, dalvastatin, eptastatin, fluindostatin, fluvastatin, lovastatin, mevastatin, pitavastatin, pravastatin, rosuvastatin, simvastatin, velostatin. In some embodiments, the statin is the hydroxyacid form of one or more of atorvastatin, cerivastatin, dalvastatin, eptastatin, fluindostatin, fluvastatin, lovastatin, mevastatin, pitavastatin, pravastatin, rosuvastatin, simvastatin, velostatin.

In some embodiments, the statin is the lactone form of one or more of lovastatin, simvastatin, and mevastatin. In some embodiments, the statin is the lactone form of lovastatin.

In some embodiments, “statin” also refers to statin analogs or derivatives which may be used in the present invention. In some embodiments, the present invention utilizes statin analogs or derivatives which include pharmaceutically acceptable esters, prodrugs, salts, solvates, enantiomers, stereoisomers, active metabolites, co-crystals, and other physiologically functional derivatives of statins. In an embodiment, the present invention contemplates the use of lovastatin analogs or derivatives. Illustrative lovastatin analogs or derivatives are described, for example, in International Patent Application No. PCT/US2016/025214, the entire contents of which are hereby incorporated by reference. In various embodiments, any of these statin analogs or derivatives are in the lactone form (e.g. substantially in the lactone for, or in an equilibrium in which the lactone form is predominant of the beta-hydroxy form), where applicable.

In various embodiments, methods are provided to identify statin analogs or derivatives that exhibit improved binding to methanogenesis-related F₄₂₀-dependent enzymes. In various embodiments, methods are provided to identify statin analogs of derivatives that exhibit improved binding to methanogenesis-related F₄₂₀-dependent enzymes such as mtd/A5UMI1 (F₄₂₀-dependent methylenetetrahydromethanopterin dehydrogenase). In some embodiments, the statin analogs or derivatives bind to and inhibit the activity of the methanogenesis-related F₄₂₀-dependent enzyme such as mtd/A5UMI1 (F₄₂₀-dependent methylenetetrahydromethanopterin dehydrogenase). In some embodiments, the statin analogs or derivatives identified through methods of the invention are utilized as agents for inhibiting methanogenesis. In some embodiments, the statin analogs or derivatives identified through methods of the invention reduces or eliminates the production and/or accumulation of methane in the GI tract.

In some embodiments, the statin analogs or derivatives are identified by using high-throughput screening. In an embodiment, the statin analogs or derivatives are derived from chemical modifications of statins and statin analogs or derivative known in the art such as those disclosed herein, including those disclosed in International Patent Application No. PCT/US2016/025214, the entire contents of which are hereby incorporated by reference. In another embodiment, the statin analogs or derivatives are derived from chemical libraries. For example, combinatorial chemistry may be employed along with high-throughput screen to identify such statin analogs or derivatives.

In an illustrative embodiment, binding assays such as competitive binding assays may be performed as part of the high-throughput screening to identify statin analogs or derivatives that bind to F₄₂₀-dependent enzymes such as mtd/A5UMI1 (F₄₂₀-dependent methylenetetrahydromethanopterin dehydrogenase) with high affinity. For example, competitive binding assays may be performed to identify statin analogs or derivatives that bind to a F₄₂₀ enzyme with higher affinity than a lactone form of lovastatin and thus displaces the lactone form of lovastatin from the binding pocket of the F₄₂₀-dependent enzyme. In various embodiments, the statin analogs or derivatives bind methanogenesis-related F₄₂₀-dependent enzymes such as mtd/A5UMI1 (F₄₂₀-dependent methylenetetrahydromethanopterin dehydrogenase) with a K_{D} of less than about 500 μM, or about 100 μM, or about 10 μM, or about 1 μM, or about 900 nM, or about 800 nM, or about 700 nM, or about 600 nM, or about 500 nM, or about 400 nM, or about 300 nM, or about 200 nM, or about 100 nM, or about 90 nM, or about 80 nM, or about 70 nM, or about 60 nM, or about 50 nM, or about 40 nM, or about 30 nM, or about 20 nM, or about 10 nM, or about 9 nM, or about 8 nM, or about 7 nM, or about 6 nM, or about 5 nM, or about 4 nM, or about 3 nM, or about 2 nM, or about 1 nM.

In various embodiments, the statin or statin analogs or derivatives effectively inhibit the activity of the methanogenesis-related F₄₂₀-dependent enzymes. The inhibitory activity of the statin, statin analogs or derivatives may be evaluated using methods known in the art. In various embodiments, the statin or statin analogs or derivatives reduce the activity of the F₄₂₀-dependent enzymes, e.g. to about 75%, or about 70%, or about 60% or about 50%, or about 40%, or about 30%, or about 25%, or about 20%, or about 10%, or about 5% of the activity in the absence of statin or statin analogs or derivatives.

In some embodiments, the statin or statin analogs or derivatives effectively inhibit the activity of a methanogenesis-related enzyme such mtd/A5UMI1 to about 75%, or about 70%, or about 60% or about 50%, or about 40%, or about 30%, or about 25%, or about 20%, or about 10%, or about 5% of the activity in the absence of the statin or statin analogs or derivatives.

Solvate as used herein refers to a pharmaceutically acceptable solvate form of a specified therapeutic agent that retains the biological effectiveness of such agent. Examples of solvates include therapeutic agents of the invention in combination with, for example, water, isopropanol, ethanol, methanol, DMSO, ethyl acetate, acetic acid, or ethanolamine.

Prodrug, as used herein refers to a therapeutic agent that is converted under physiological conditions or by solvolysis or metabolically (e.g., in vivo) to a specified agent that is pharmaceutically active.

Active metabolite, as used herein refers to a pharmacologically active product produced through metabolism in the body of a specified therapeutic agent.

Co-crystal as used herein refers to a physical association of two or more molecules which owe their stability through non-covalent interaction. One or more components of this molecular complex provide a stable framework in the crystalline lattice. In certain instances, the guest molecules are incorporated in the crystalline lattice as anhydrates or solvates.

Additional Compounds that Modulate F₄₂₀-Dependent Enzymes

The present invention provides novel compounds that bind to and modulate the activities of methanogenesis-related F₄₂₀-dependent enzymes.

In various embodiments, methods are provided to identify compounds that exhibit improved binding to methanogenesis-related F₄₂₀-dependent enzymes. In some embodiments, methods are provided to identify compounds that exhibit improved binding to methanogenesis-related enzymes that utilizes F₄₂₀ as a coenzyme. In various embodiments, methods are provided to identify compounds that exhibit improved binding to methanogenesis-related F₄₂₀-dependent enzymes such as mtd/A5UMI1 (F₄₂₀-dependent methylenetetrahydromethanopterin dehydrogenase).

In some embodiments, the present invention provides compounds that bind to and inhibit the activity of a methanogenesis-related F₄₂₀-dependent enzyme such as mtd/A5UMI1 (F₄₂₀-dependent methylenetetrahydromethanopterin dehydrogenase). In some embodiments, the compounds identified through methods of the invention are utilized as agents for inhibiting methanogenesis. In some embodiments, the compounds identified through methods of the invention reduce or eliminate the production and/or accumulation of methane in the GI tract.

In some embodiments, the compounds are identified by using any of the binding assays or high-throughput screening described herein. In an embodiment, the compounds of the invention are identified using a Pharmacophoric Fingerprinting (PFP) methodology, as described, for example, in McGregor et al. (1999) J. Chem. Inf. Comput. Sci. 39:569-574 and McGregor et al. (2000) J. Chem. Inf. Comput Sci. 40:117-125, the entire contents of which are hereby incorporated by reference.

In various embodiments, the present invention provides compounds that bind to and modulate the activities of methanogenesis-related F₄₂₀-dependent enzymes.

In various embodiments, the present invention provides a compound having the structure of Formula I:

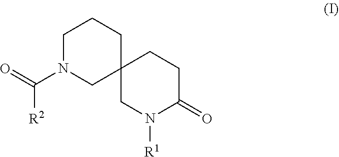

or pharmaceutically acceptable salts, stereoisomers, or prodrugs derivatives thereof, wherein: R¹ is selected from C₁-C₆ alkyl, hydroxy-C₁-C₆ alkyl, (C₃-C₆ cycloalkyl)-C₁-C₃ alkyl, and C₃-C₆ cycloalkyl; R² is selected from R^{hc}, —CH₂—R^{hc}, —CH₂CH₂—R^{hc}, C₃-C₆ cycloalkyl (optionally substituted with C₁-C₃ alkyl or hydroxy-C₁-C₃ alkyl), heteroalkyl (optionally substituted with one or more moieties independently selected from oxo, amino (—NH₂), (C₁-C₃ alkyl)amino, and di(C₁-C₃ alkyl)amino); and R^{hc} is selected from 5- or 6-membered heterocyclic ring (optionally substituted with one or more moieties independently selected from oxo, tetrazolyl, C₃-C₆ cycloalkyl, hydroxy-C₁-C₃ alkyl, C₁-C₃ alkyl, and amino (—NH₂)), heterocyclic ring system (optionally substituted with oxo, amino (—NH₂), (C₁-C₃ alkyl)amino, and di(C₁-C₃ alkyl)amino)).

In various embodiments, the compound is:

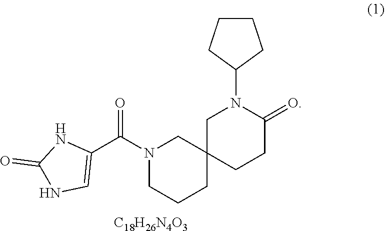 [SMILES: O=C(c1c[nH]c(=O)[nH]1)N1CCCC2(CCC(=O)N(C3CCCC3)C2)C1 ; "C18H26N4O3"]

In various embodiments, the compound is:

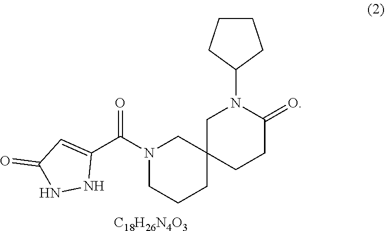 [SMILES: O=C(c1cc(=O)[nH][nH]1)N1CCCC2(CCC(=O)N(C3CCCC3)C2)C1 ; "C18H26N4O3"]

In various embodiments, the compound is:

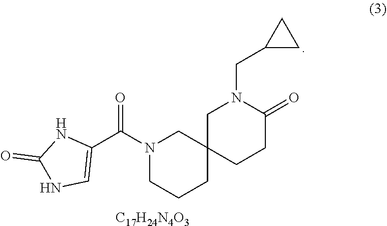

In various embodiments, the compound is:

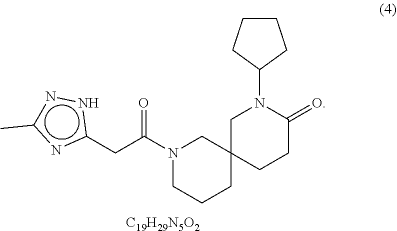

In various embodiments, the compound is:

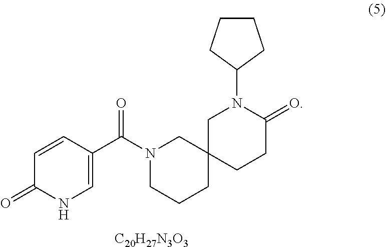 [SMILES: O=C(c1ccc(=O)[nH]c1)N1CCCC2(CCC(=O)N(C3CCCC3)C2)C1 ; "C20H27N3O3"]

In various embodiments, the compound is:

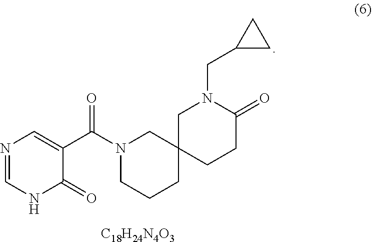

In various embodiments, the compound is:

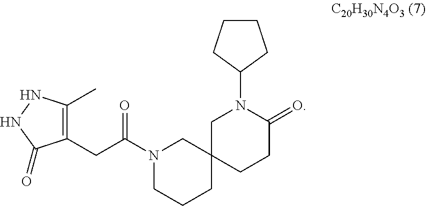 [SMILES: Cc1[nH][nH]c(=O)c1CC(=O)N1CCCC2(CCC(=O)N(C3CCCC3)C2)C1]

In various embodiments, the compound is:

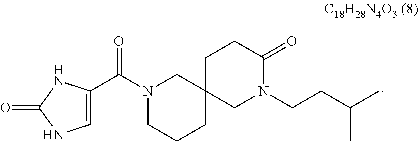 [SMILES: CC(C)CCN1CC2(CCCN(C(=O)c3c[nH]c(=O)[nH]3)C2)CCC1=O]

In various embodiments, the compound is:

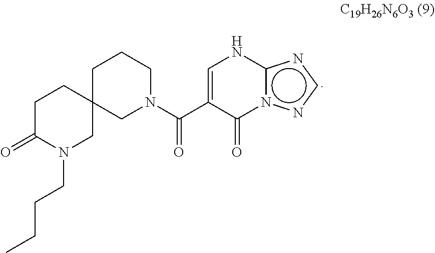

In various embodiments, the compound is:

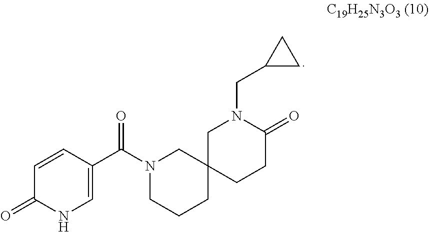 [SMILES: O=C1CCC2(CCCN(C(=O)c3ccc(=O)[nH]c3)C2)CN1CC1CC1]

In various embodiments, the compound is:

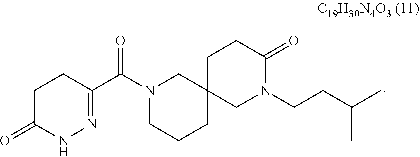 [SMILES: CC(C)CCN1CC2(CCCN(C(=O)C3=NNC(=O)CC3)C2)CCC1=O]

In various embodiments, the compound is:

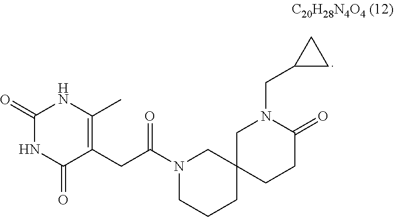 [SMILES: Cc1[nH]c(=O)[nH]c(=O)c1CC(=O)N1CCCC2(CCC(=O)N(CC3CC3)C2)C1]

In various embodiments, the compound is:

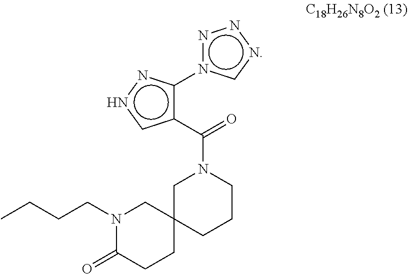

In various embodiments, the compound is:

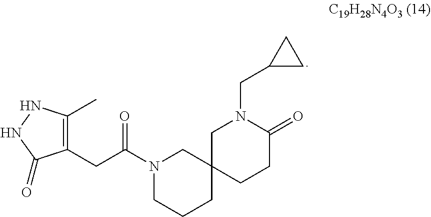 [SMILES: Cc1[nH][nH]c(=O)c1CC(=O)N1CCCC2(CCC(=O)N(CC3CC3)C2)C1]

In various embodiments, the compound is:

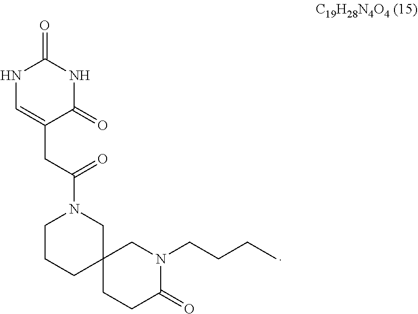 [SMILES: CCCCN1CC2(CCCN(C(=O)Cc3c[nH]c(=O)[nH]c3=O)C2)CCC1=O]

In various embodiments, the compound is:

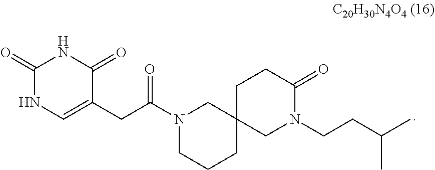 [SMILES: CC(C)CCN1CC2(CCCN(C(=O)Cc3c[nH]c(=O)[nH]c3=O)C2)CCC1=O]

In various embodiments, the compound is:

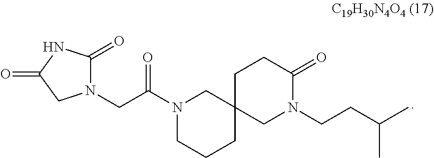 [SMILES: CC(C)CCN1CC2(CCCN(C(=O)CN3CC(=O)NC3=O)C2)CCC1=O]

In various embodiments, the compound is:

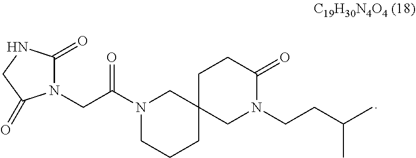 [SMILES: CC(C)CCN1CC2(CCCN(C(=O)CN3C(=O)CNC3=O)C2)CCC1=O]

In various embodiments, the compound is:

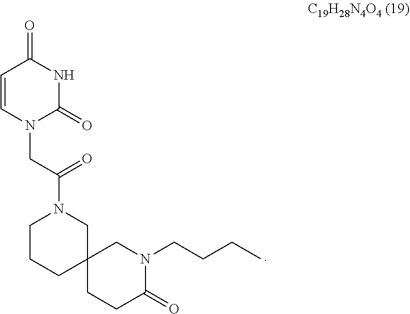 [SMILES: CCCCN1CC2(CCCN(C(=O)Cn3ccc(=O)[nH]c3=O)C2)CCC1=O]

In various embodiments, the compound is:

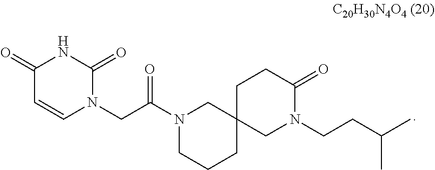 [SMILES: CC(C)CCN1CC2(CCCN(C(=O)Cn3ccc(=O)[nH]c3=O)C2)CCC1=O]

In various embodiments, the compound is:

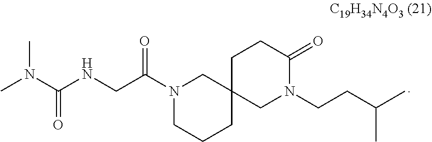 [SMILES: CC(C)CCN1CC2(CCCN(C(=O)CNC(=O)N(C)C)C2)CCC1=O]

In various embodiments, the compound is:

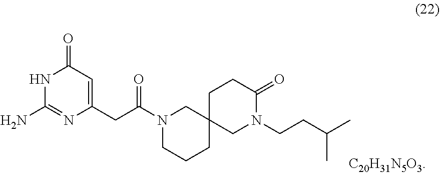 [SMILES: CC(C)CCN1CC2(CCCN(C(=O)Cc3cc(=O)[nH]c(N)n3)C2)CCC1=O]

In various embodiments, the compound is:

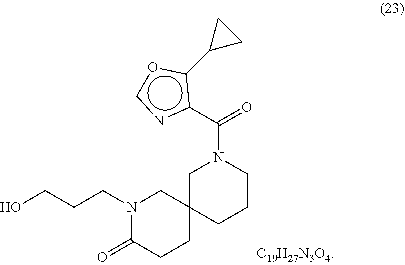

In various embodiments, the compound is:

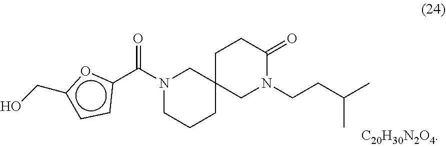

In various embodiments, the compound is:

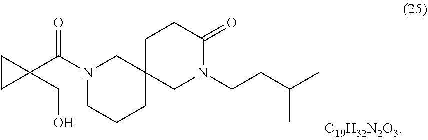 [SMILES: CC(C)CCN1CC2(CCCN(C(=O)C3(CO)CC3)C2)CCC1=O]

In various embodiments, the compound is:

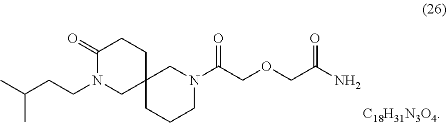 [SMILES: CC(C)CCN1CC2(CCCN(C(=O)COCC(N)=O)C2)CCC1=O]

In an embodiment, the present invention provides a compound having the structure of one of Formula IIa-IIc:

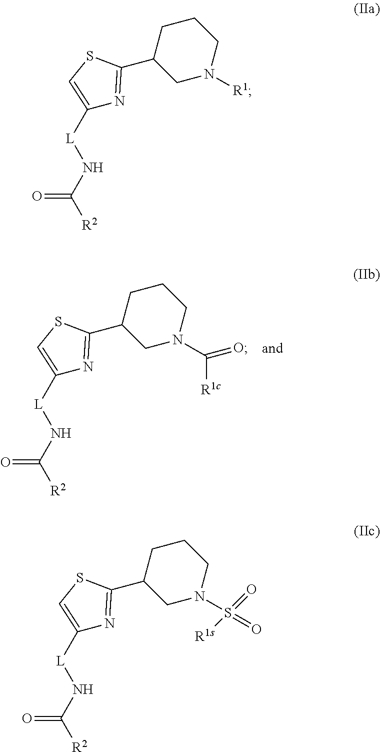

or pharmaceutically acceptable salts, stereoisomers, or prodrugs derivatives thereof, wherein: L is a bond selected from —CH₂— and —CH₂CH₂—; R¹ is selected from —C(O)R^{1c} and —SO₂R^{1s}; R^{1c} is selected from C₁-C₆ alkyl, C₃-C₇ cycloalkyl-C₁-C₃ alkyl, C₂-C₆ alkenyl, and C₃-C₇ cycloalkyl; R^{1s} is C₁-C₆ alkyl, C₃-C₇ cycloalkyl-C₁-C₃ alkyl, C₂-C₆ alkenyl, and C₃-C₇ cycloalkyl; and R² is selected from C₁-C₆ alkyl and C₃-C₆ cycloalkyl.

In various embodiments, the compound is:

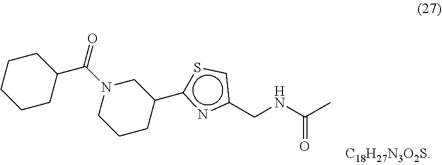

In various embodiments, the compound is:

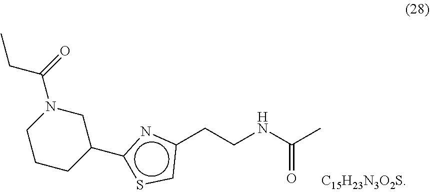

In various embodiments, the compound is:

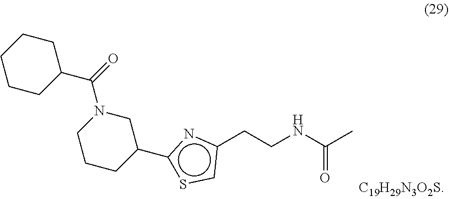

In various embodiments, the compound is:

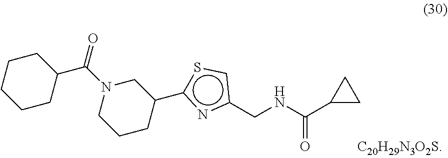

In various embodiments, the compound is:

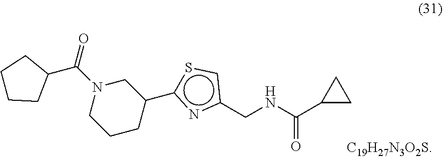

In various embodiments, the compound is:

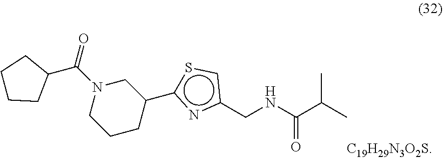

In various embodiments, the compound is:

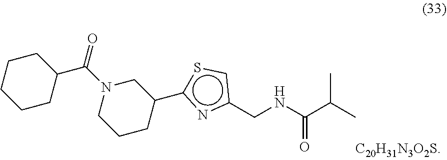

In various embodiments, the compound is:

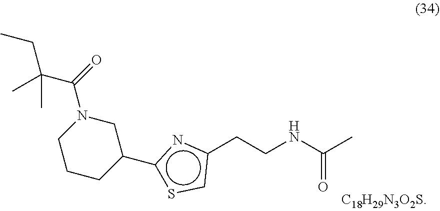

In various embodiments, the compound is:

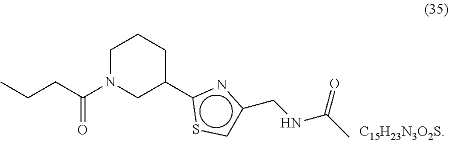

In various embodiments, the compound is:

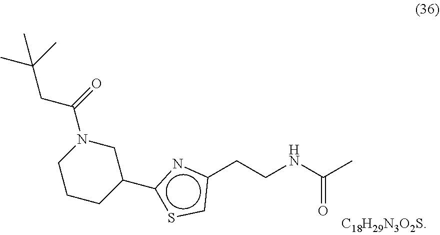

In various embodiments, the compound is:

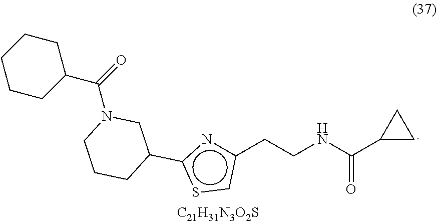

In various embodiments, the compound is:

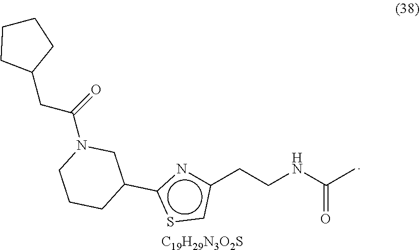

In various embodiments, the compound is:

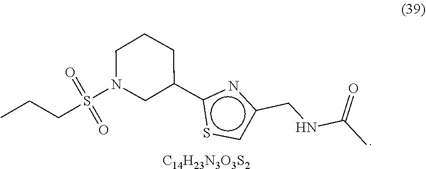

In various embodiments, the compound is:

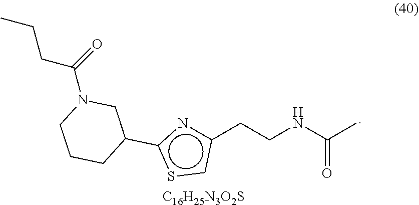

In various embodiments, the compound is:

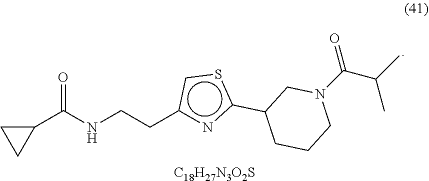

In various embodiments, the compound is:

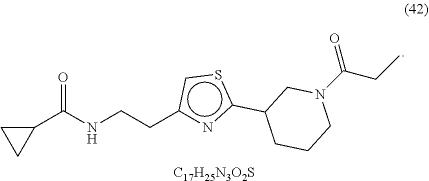

In various embodiments, the compound is:

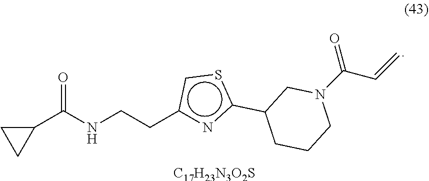

In various embodiments, the compound is:

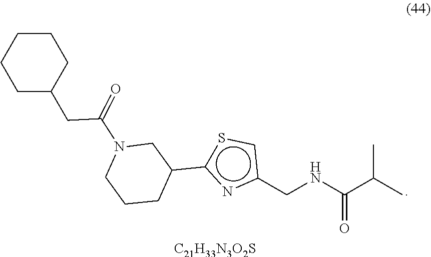

In various embodiments, the compound is:

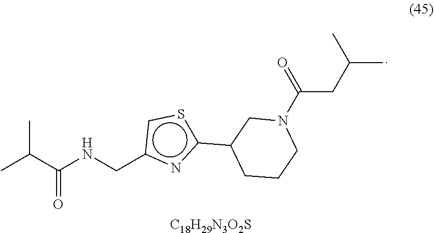

In various embodiments, the compound is:

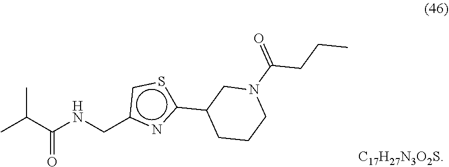

In various embodiments, the compound is:

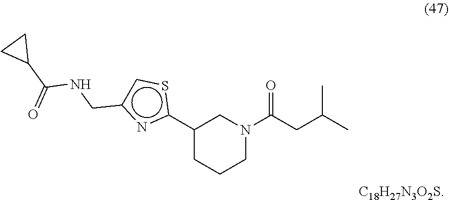

In various embodiments, the compound is:

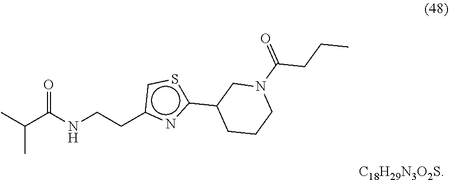

In various embodiments, the compound is:

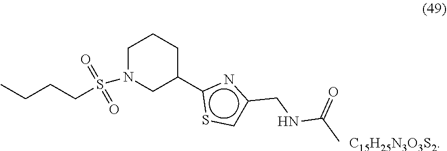

In various embodiments, the compound is:

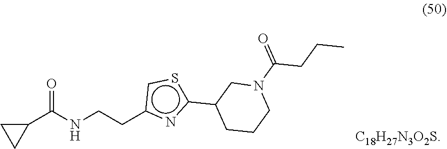

In various embodiments, the compound is:

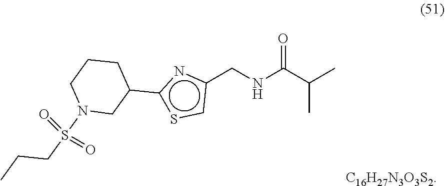

In various embodiments, the compound is:

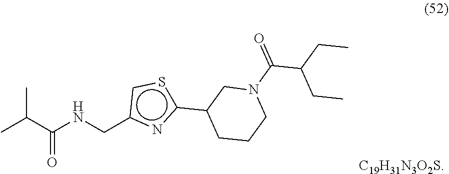

In various embodiments, the compound is:

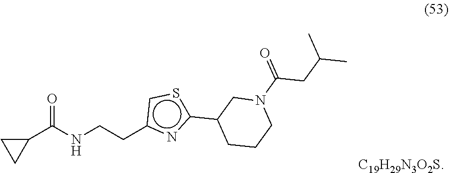

In various embodiments, the compound is:

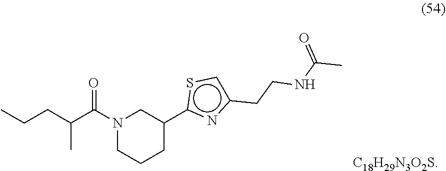

In various embodiments, the compound is:

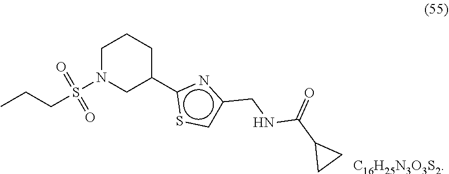

In various embodiments, the compound is:

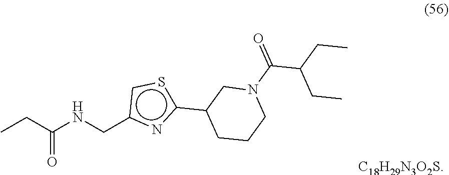

In an embodiment, the present invention provides a compound having the structure of Formula III:

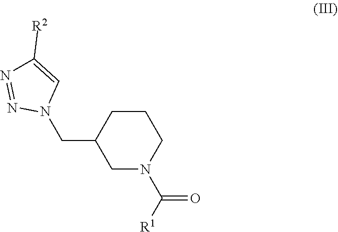

or pharmaceutically acceptable salts, stereoisomers, or prodrugs derivatives thereof, wherein R¹ is selected from C₅-C₈ cycloalkyl and 5- or 6-membered heterocyclic ring (optionally substituted with one or more moieties independently selected from oxo, C₁-C₃ alkyl, and amino (—NH₂)); and R² is selected from C₁-C₆ alkyl and hydroxy-C₁-C₆ alkyl.

In various embodiments, the compound is:

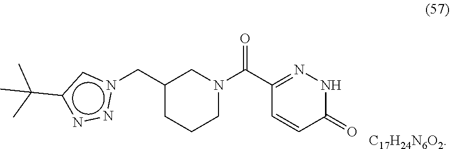

In various embodiments, the compound is:

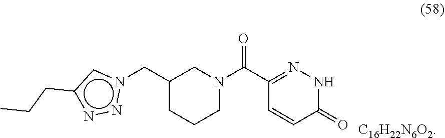

In various embodiments, the compound is:

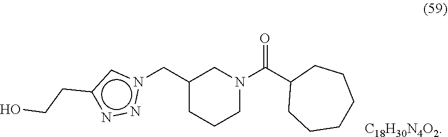

In various embodiments, the compound is:

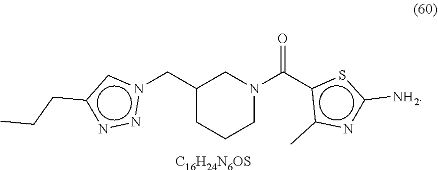

In various embodiments, the compound is:

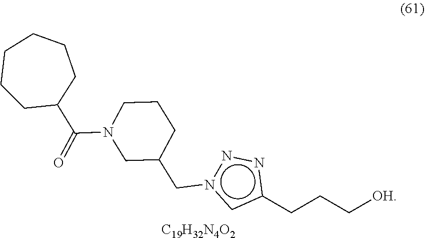

In an embodiment, the present invention provides a compound having the structure of Formula IV:

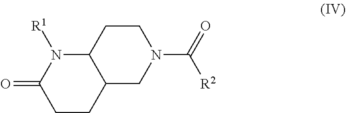

or pharmaceutically acceptable salts, stereoisomers, or prodrugs derivatives thereof, wherein: R¹ is selected from C₁-C₆ alkyl, hydroxy-C₁-C₆ alkyl, and (C₁-C₃ alkyl)thio-C₁-C₆ alkyl; R² is selected from R^{hc}, —CH₂—R^{hc}, —CH₂CH₂—R^{hc}, C₃-C₆ cycloalkyl (optionally substituted with carbamoyl (—C(O)NH₂) or N—(C₁-C₃ alkyl)-carbamoyl), C₂-C₆ alkenyl, C₁-C₆ alkyl (optionally substituted with (C₁-C₃ alkyl)sulfonamido (—NHSO₂(C₁-C₃ alkyl)), sulfamoyl (—SO₂NH₂), or N—(C₁-C₃ alkyl)-sulfamoyl), and (C₁-C₃ alkyl)thio-C₁-C₆ alkyl; and R^{hc} is a 5- or 6-membered heterocyclic ring (optionally substituted with one or more moieties independently selected from oxo and C₁-C₃ alkyl).

In various embodiments, the compound is:

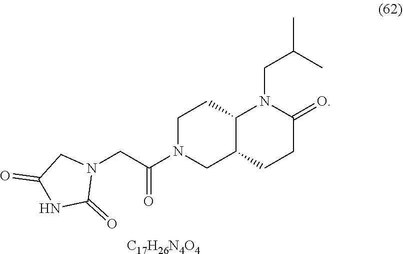

In various embodiments, the compound is:

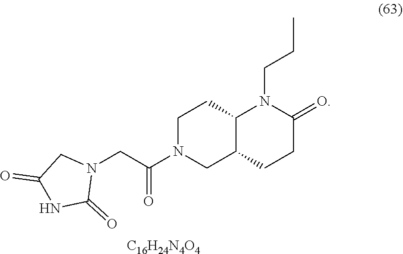

In various embodiments, the compound is:

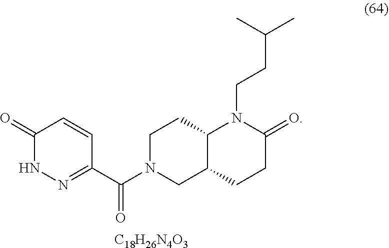

In various embodiments, the compound is:

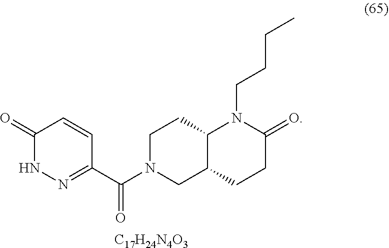

In various embodiments, the compound is:

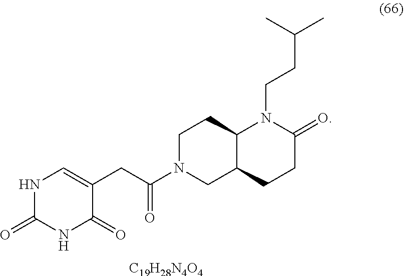

In various embodiments, the compound is:

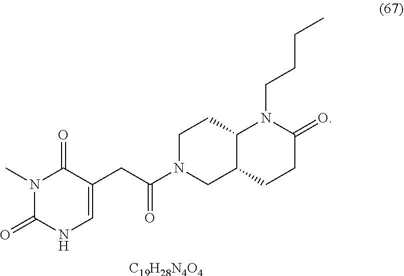 [SMILES: CCCCN1C(=O)CC[C@@H]2CN(C(=O)Cc3c[nH]c(=O)n(C)c3=O)CC[C@@H]21 ; "C19H28N4O4"]

In various embodiments, the compound is:

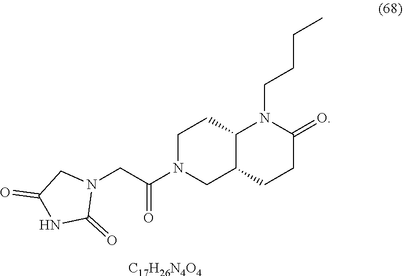

In various embodiments, the compound is:

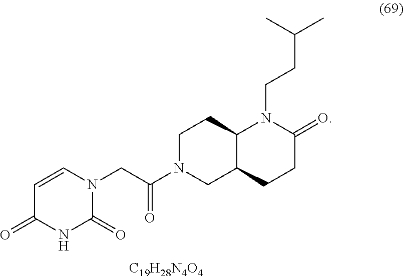

In various embodiments, the compound is:

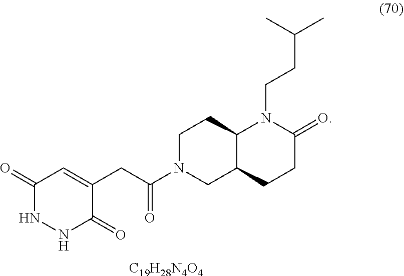

In various embodiments, the compound is:

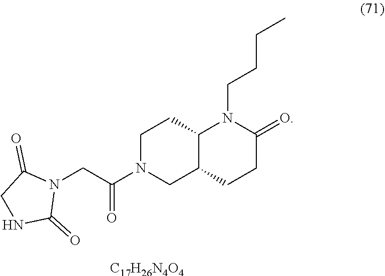 [SMILES: CCCCN1C(=O)CC[C@@H]2CN(C(=O)CN3C(=O)CNC3=O)CC[C@@H]21 ; "C17H26N4O4"]

In various embodiments, the compound is:

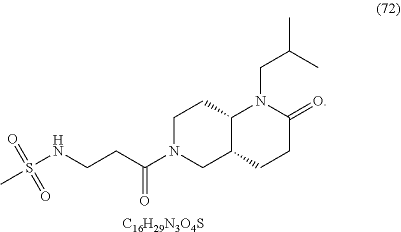

In various embodiments, the compound is:

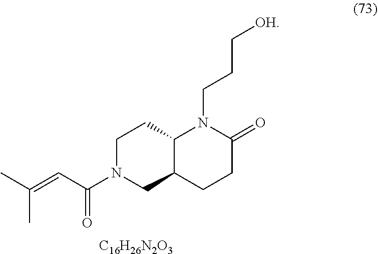

In various embodiments, the compound is:

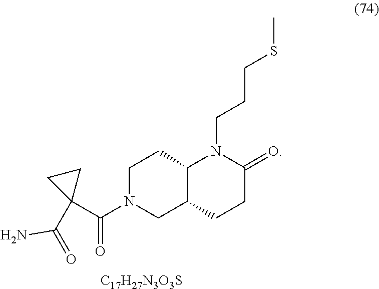

In various embodiments, the compound is:

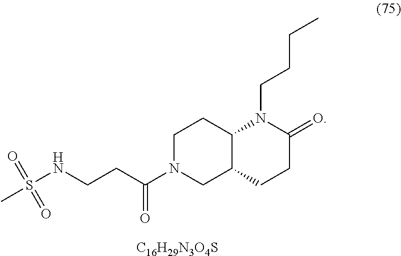

In various embodiments, the compound is:

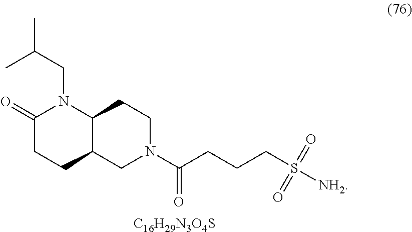

In various embodiments, the compound is:

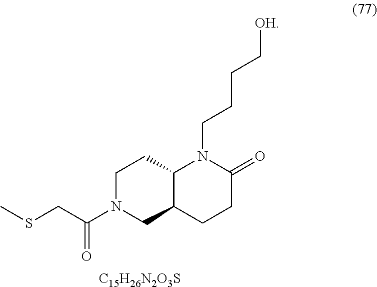

In an embodiment, the present invention provides a compound having the structure of Formula V:

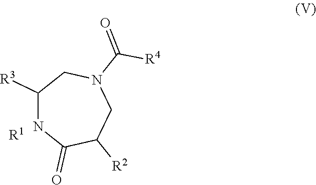

or pharmaceutically acceptable salts, stereoisomers, or prodrugs derivatives thereof, wherein: R¹ is selected from (C₃-C₆ cycloalkyl)-C₁-C₃ alkyl and C₂-C₆ alkenyl; R² is H and R³ is C₁-C₆ alkyl; or R² and R³ are joined to form a C₁-C₆ alkyl bridge; R⁴ is selected from —R^{hc}, —CH₂—R^{hc}, and —CH₂CH₂—R^{hc}; and R^{hc} is a 5- or 6-membered heterocyclic ring (optionally substituted with one or more moieties independently selected from oxo, amino (—NH₂), C₁-C₃ alkyl, and hydroxy).

In various embodiments, the compound is:

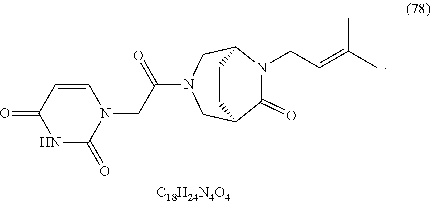 [SMILES: CC(C)=CCN1C(=O)[C@@H]2CC[C@H]1CN(C(=O)Cn1ccc(=O)[nH]c1=O)C2 ; "C18H24N4O4"]

In various embodiments, the compound is:

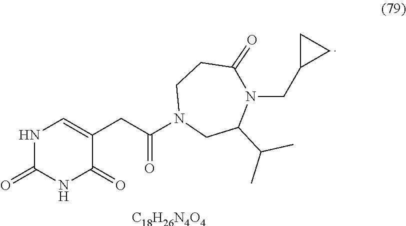

In various embodiments, the compound is:

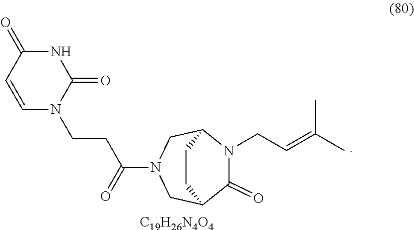

In various embodiments, the compound is:

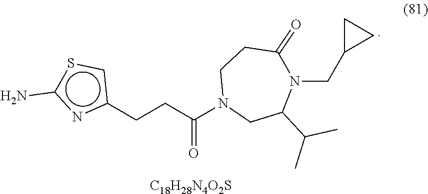

In various embodiments, the compound is:

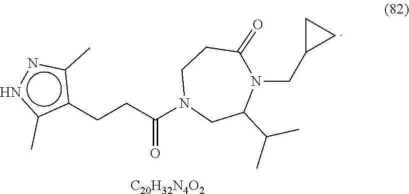

In various embodiments, the compound is:

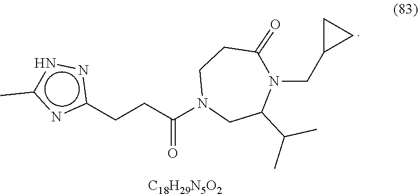

In various embodiments, the compound is:

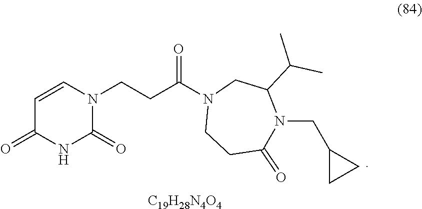 [SMILES: CC(C)C1CN(C(=O)CCn2ccc(=O)[nH]c2=O)CCC(=O)N1CC1CC1 ; "C19H28N4O4"]

In various embodiments, the compound is:

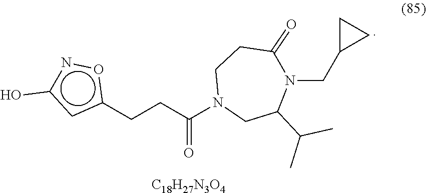

In various embodiments, the compound is:

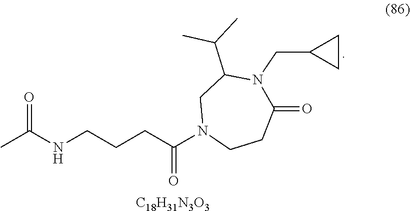

In an embodiment, the present invention provides a compound having the structure of Formula VI:

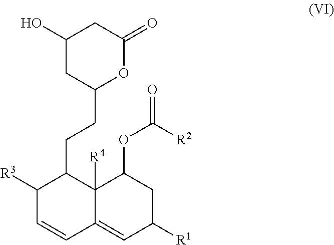

or pharmaceutically acceptable salts, stereoisomers, or prodrugs derivatives thereof, wherein: R¹ is selected from H, C₁-C₃ alkyl, =CH₂, =CHCH₃, —C(CH₃)₂, and =CHCH₂CH₃; R² is selected from C₃-C₆ cycloalkyl (optionally substituted with C₁-C₃ alkyl), C₁-C₆ alkyl, and halo-C₁-C₆ alkyl; (e.g. “haloalkyl” as alkyl with one or more halogen atoms, such as, by way of non-limitation, difluoro-C₁-C₆ alkyl); R³ is selected from H and C₁-C₃ alkyl; and R⁴ is selected from H, and C₁-C₃ alkyl.

In various embodiments, the compound is:

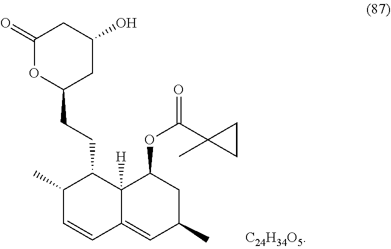 [SMILES: C[C@H]1C=C2C=C[C@H](C)[C@H](CC[C@@H]3C[C@@H](O)CC(=O)O3)[C@H]2[C@@H](OC(=O)C2(C)CC2)C1]

In various embodiments, the compound is:

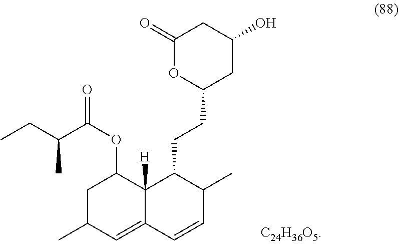 [SMILES: CC[C@H](C)C(=O)OC1CC(C)C=C2C=CC(C)[C@@H](CC[C@H]3C[C@@H](O)CC(=O)O3)[C@H]21]

In various embodiments, the compound is:

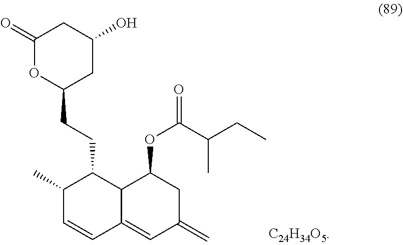 [SMILES: C=C1C=C2C=C[C@H](C)[C@H](CC[C@@H]3C[C@@H](O)CC(=O)O3)C2[C@@H](OC(=O)C(C)CC)C1]

In various embodiments, the compound is:

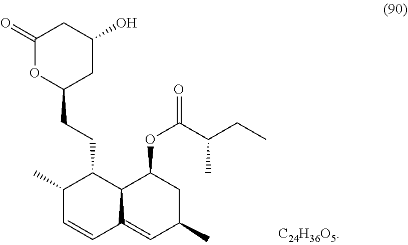 [SMILES: CC[C@H](C)C(=O)O[C@H]1C[C@@H](C)C=C2C=C[C@H](C)[C@H](CC[C@@H]3C[C@@H](O)CC(=O)O3)[C@H]21]

In various embodiments, the compound is:

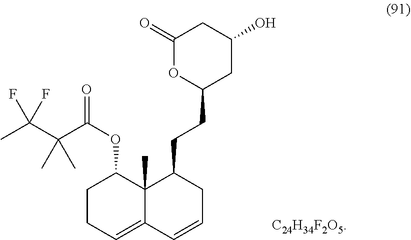 [SMILES: CC(F)(F)C(C)(C)C(=O)O[C@H]1CCC=C2C=CC[C@H](CC[C@@H]3C[C@@H](O)CC(=O)O3)[C@]21C]

In various embodiments, the compound is:

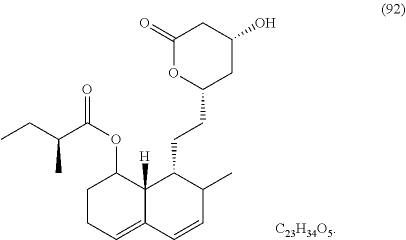 [SMILES: CC[C@H](C)C(=O)OC1CCC=C2C=CC(C)[C@@H](CC[C@H]3C[C@@H](O)CC(=O)O3)[C@H]21]

In various embodiments, the compound is:

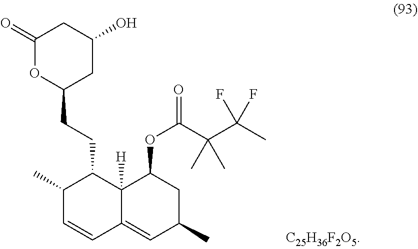 [SMILES: C[C@H]1C=C2C=C[C@H](C)[C@H](CC[C@@H]3C[C@@H](O)CC(=O)O3)[C@H]2[C@@H](OC(=O)C(C)(C)C(C)(F)F)C1]

In various embodiments, the compound is:

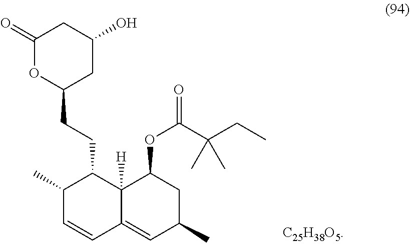 [SMILES: CCC(C)(C)C(=O)O[C@H]1C[C@@H](C)C=C2C=C[C@H](C)[C@H](CC[C@@H]3C[C@@H](O)CC(=O)O3)[C@H]21]

In various embodiments, the compound is:

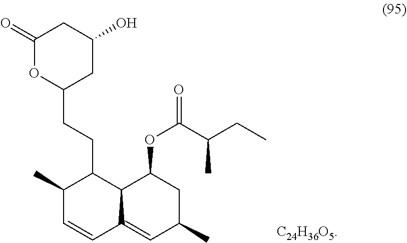

In various embodiments, the compound is:

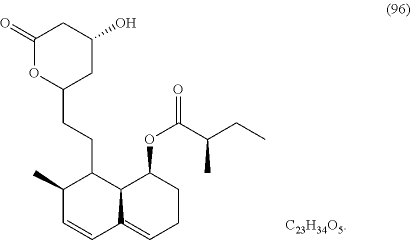

In various embodiments, the compound is:

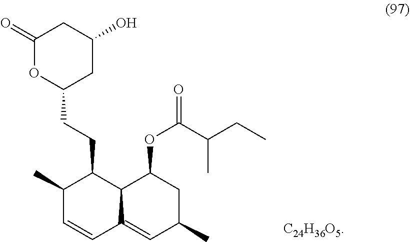 [SMILES: CCC(C)C(=O)O[C@H]1C[C@@H](C)C=C2C=C[C@@H](C)[C@@H](CC[C@H]3C[C@@H](O)CC(=O)O3)[C@H]21]

In various embodiments, the compound is:

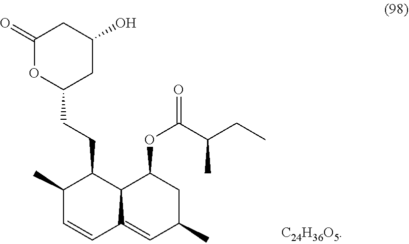 [SMILES: CC[C@@H](C)C(=O)O[C@H]1C[C@@H](C)C=C2C=C[C@@H](C)[C@@H](CC[C@H]3C[C@@H](O)CC(=O)O3)[C@H]21]

In various embodiments, the compound is:

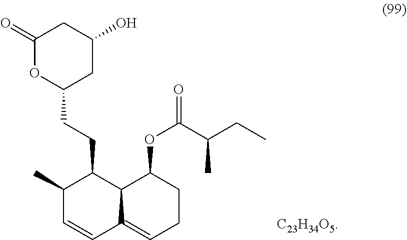 [SMILES: CC[C@@H](C)C(=O)O[C@H]1CCC=C2C=C[C@@H](C)[C@@H](CC[C@H]3C[C@@H](O)CC(=O)O3)[C@H]21]

In various embodiments, the compound is:

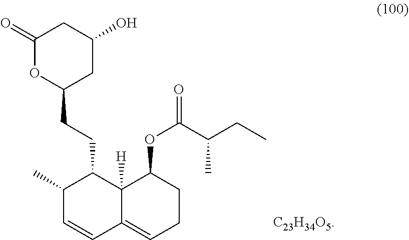 [SMILES: CC[C@H](C)C(=O)O[C@H]1CCC=C2C=C[C@H](C)[C@H](CC[C@@H]3C[C@@H](O)CC(=O)O3)[C@H]21]

In various embodiments, the compound is:

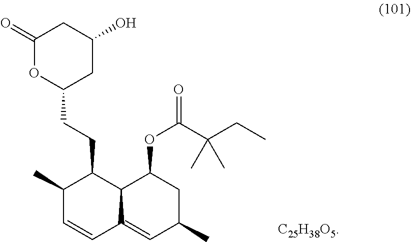 [SMILES: CCC(C)(C)C(=O)O[C@H]1C[C@@H](C)C=C2C=C[C@@H](C)[C@@H](CC[C@H]3C[C@@H](O)CC(=O)O3)[C@H]21]

In various embodiments, the compound is:

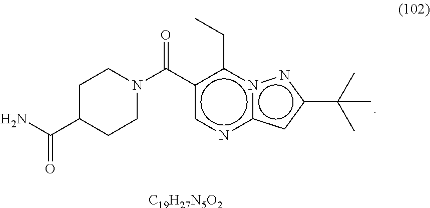

In various embodiments, the compound is:

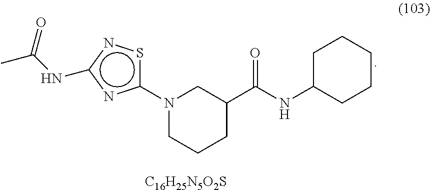

In various embodiments, the compound is:

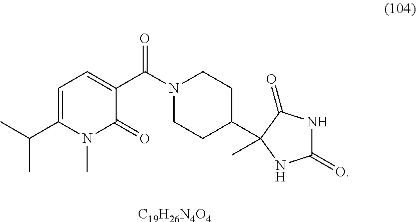 [SMILES: CC(C)c1ccc(C(=O)N2CCC(C3(C)NC(=O)NC3=O)CC2)c(=O)n1C ; "C19H26N4O4"]

In various embodiments, the compound is:

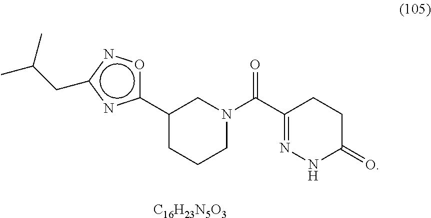

In various embodiments, the compound is:

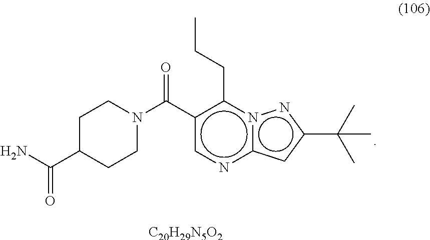

In various embodiments, the compound is:

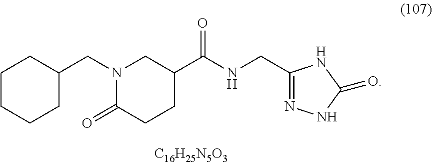 [SMILES: O=C(NCc1n[nH]c(=O)[nH]1)C1CCC(=O)N(CC2CCCCC2)C1 ; "C16H25N5O3"]

In various embodiments, the compound is:

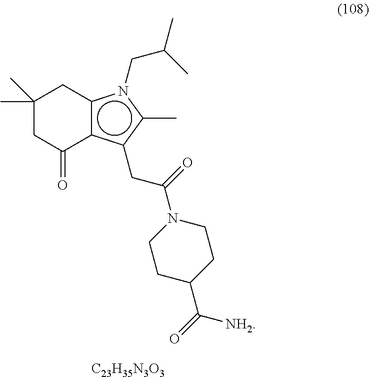

In various embodiments, the compound is:

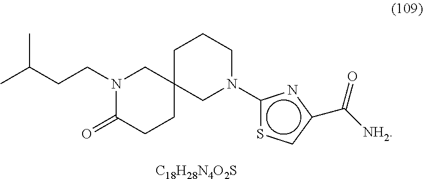

In various embodiments, the compound is:

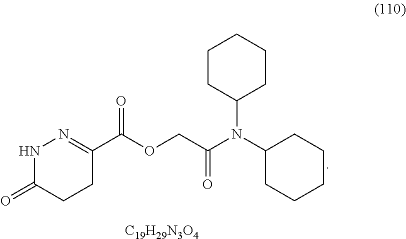 [SMILES: O=C1CCC(C(=O)OCC(=O)N(C2CCCCC2)C2CCCCC2)=NN1 ; "C19H29N3O4"]

In various embodiments, the compound is:

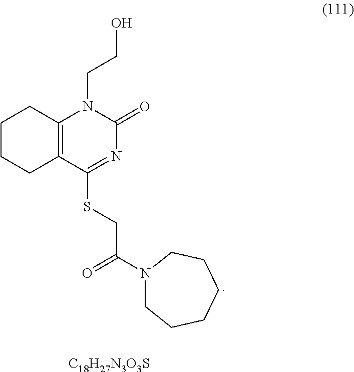 [SMILES: O=C(CSc1nc(=O)n(CCO)c2c1CCCC2)N1CCCCCC1 ; "C18H27N3O3S"]

In various embodiments, the compound is:

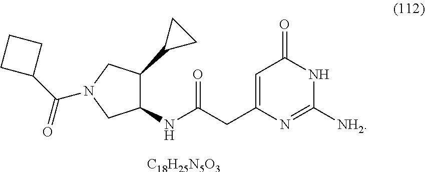 [SMILES: Nc1nc(CC(=O)N[C@H]2CN(C(=O)C3CCC3)C[C@H]2C2CC2)cc(=O)[nH]1 ; "C18H25N5O3"]

In various embodiments, the compound is:

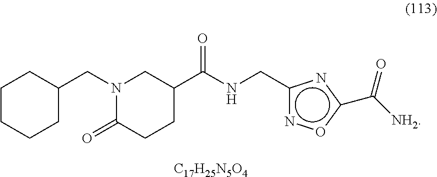

In various embodiments, the compound is:

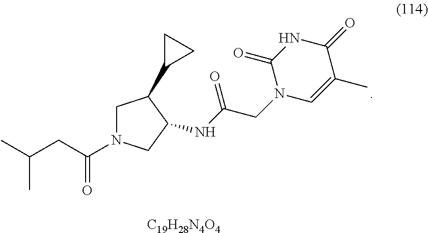 [SMILES: Cc1cn(CC(=O)N[C@@H]2CN(C(=O)CC(C)C)C[C@H]2C2CC2)c(=O)[nH]c1=O ; "C19H28N4O4"]

In various embodiments, the compound is:

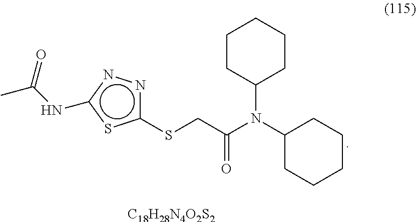

In various embodiments, the compound is:

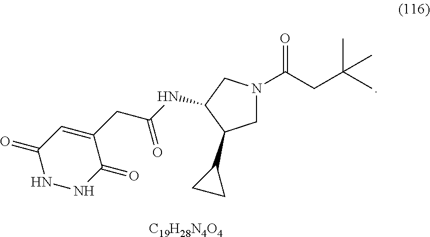 [SMILES: CC(C)(C)CC(=O)N1C[C@@H](NC(=O)Cc2cc(=O)[nH][nH]c2=O)[C@H](C2CC2)C1 ; "C19H28N4O4"]

In various embodiments, the compound is:

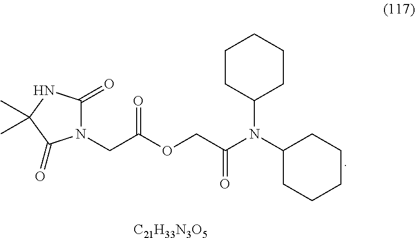 [SMILES: CC1(C)NC(=O)N(CC(=O)OCC(=O)N(C2CCCCC2)C2CCCCC2)C1=O ; "C21H33N3O5"]

In various embodiments, the compound is:

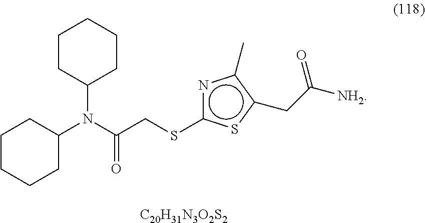

In various embodiments, the compound is:

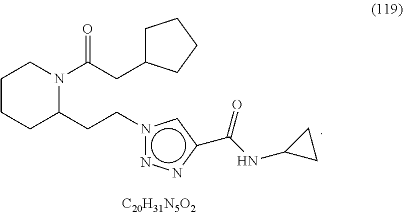

In various embodiments, the compound is:

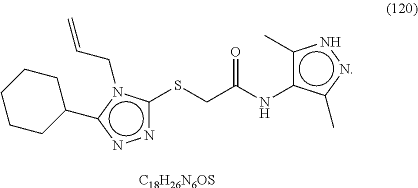

In various embodiments, the compound is:

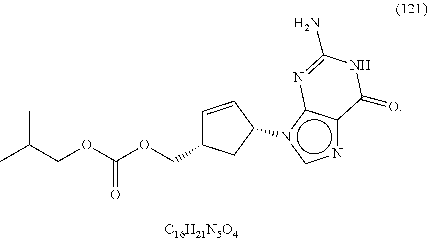

In various embodiments, the compound is:

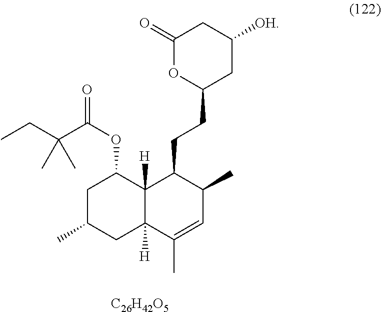 [SMILES: CCC(C)(C)C(=O)O[C@H]1C[C@@H](C)C[C@@H]2C(C)=C[C@H](C)[C@H](CC[C@@H]3C[C@@H](O)CC(=O)O3)[C@@H]12 ; "C26H42O5"]

In various embodiments, the compound is:

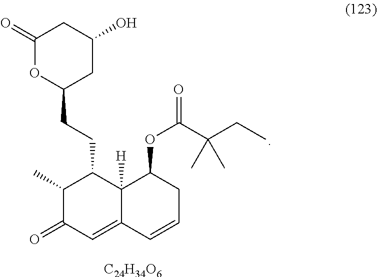

In various embodiments, the compound is:

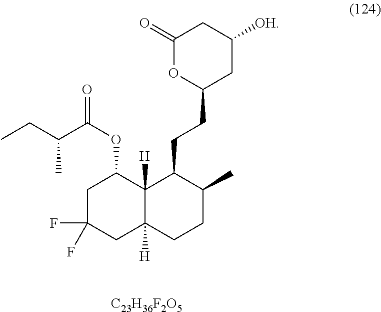 [SMILES: CC[C@@H](C)C(=O)O[C@H]1CC(F)(F)C[C@@H]2CC[C@H](C)[C@H](CC[C@@H]3C[C@@H](O)CC(=O)O3)[C@H]21 ; "C23H36F2O5"]

In various embodiments, the compound is:

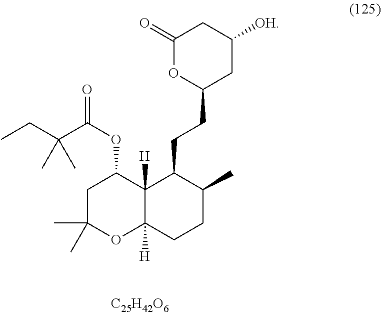 [SMILES: CCC(C)(C)C(=O)O[C@H]1CC(C)(C)O[C@@H]2CC[C@H](C)[C@H](CC[C@@H]3C[C@@H](O)CC(=O)O3)[C@@H]12 ; "C25H42O6"]

In various embodiments, the compound is:

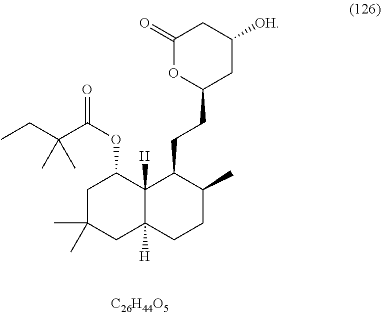 [SMILES: CCC(C)(C)C(=O)O[C@H]1CC(C)(C)C[C@@H]2CC[C@H](C)[C@H](CC[C@@H]3C[C@@H](O)CC(=O)O3)[C@H]21 ; "C26H44O5"]

In various embodiments, the compound is:

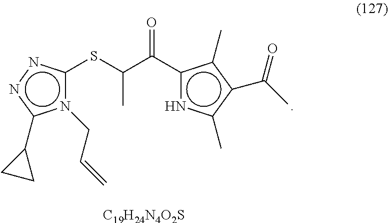

In various embodiments, the compound is:

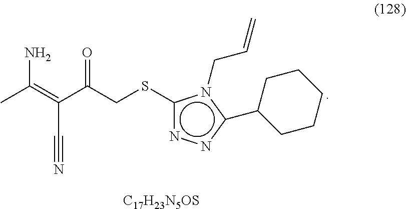

In various embodiments, the compound is:

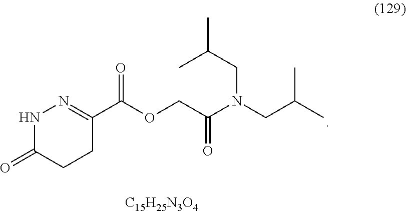 [SMILES: CC(C)CN(CC(C)C)C(=O)COC(=O)C1=NNC(=O)CC1 ; "C15H25N3O4"]

In various embodiments, the compound is:

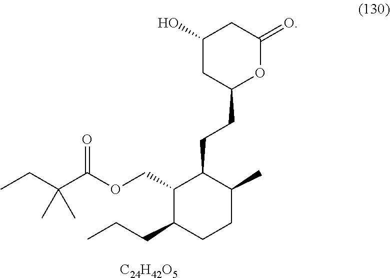

In various embodiments, the compound is:

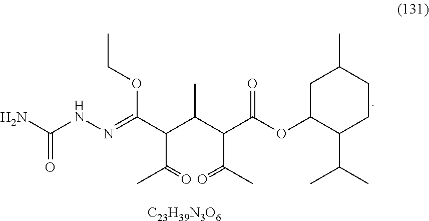

In various embodiments, the compounds of the invention exhibit enhanced binding to and/or specificity for methanogenesis-related F420-dependent enzymes compared to statins (e.g., lovastatins). In various embodiments, the compounds of the invention bind F₄₂₀-dependent enzymes such as mtd/A5UMI1 (F₄₂₀-dependent methylenetetrahydromethanopterin dehydrogenase) with a K_{D} of less than about 500 μM, or about 100 μM, or about 10 μM, or about 1 μM, or about 900 nM, or about 800 nM, or about 700 nM, or about 600 nM, or about 500 nM, or about 400 nM, or about 300 nM, or about 200 nM, or about 100 nM, or about 90 nM, or about 80 nM, or about 70 nM, or about 60 nM, or about 50 nM, or about 40 nM, or about 30 nM, or about 20 nM, or about 10 nM, or about 9 nM, or about 8 nM, or about 7 nM, or about 6 nM, or about 5 nM, or about 4 nM, or about 3 nM, or about 2 nM, or about 1 nM.

In various embodiments, the compounds the invention effectively inhibit the activity of the methanogenesis-related F₄₂₀-dependent enzymes. The inhibitory activity of the compounds of the invention may be evaluated using methods known in the art. In various embodiments, the compounds of the invention reduce the activity of the methanogenesis-related F₄₂₀-dependent enzymes, e.g. to about 75%, or about 70%, or about 60% or about 50%, or about 40%, or about 30%, or about 25%, or about 20%, or about 10%, or about 5% of the activity in the absence of the compounds.

In some embodiments, the compounds of the invention effectively inhibit the activity of a methanogenesis-related enzyme such mtd/A5UMI1 to about 75%, or about 70%, or about 60% or about 50%, or about 40%, or about 30%, or about 25%, or about 20%, or about 10%, or about 5% of the activity in the absence of the compounds.

In some embodiments, the present invention utilizes analogs or derivatives of compounds of the invention which include pharmaceutically acceptable esters, prodrugs, salts, solvates, enantiomers, stereoisomers, active metabolites, co-crystals, and other physiologically functional derivatives these compounds.

### Modified Release Profile

In one aspect, the present invention provides modified release formulations comprising at least one statin, statin analog or derivative, or compound disclosed herein, e.g. a compound modulating one or more methanogenesis-related F₄₂₀-dependent enzymes, wherein the formulation releases at least about 60% of the statin, statin analog or derivative, or compound disclosed herein after the stomach and into one or more regions of the intestinal tract.

In various embodiments, the statin, statin analog or derivative, or compound can inhibit the production of methane, inhibit methanogenesis, or inhibit the growth and/or proliferation of methanogens. In some embodiments, the statin or statin analog or derivative is in a hydroxyacid form which typically is, without wishing to be bound by theory, an effective inhibitor of HMG-CoA reductase, or in a lactone form which typically is, without wishing to be bound by theory, an ineffective HMG-CoA inhibitor.

In various embodiments, the statin or statin analog or derivative is in the lactone form, including substantially in the lactone form, at the site of delivery by the present formulations. For example, in some embodiments, the amount of GI tract-delivered statin or statin analog or derivative which is in the lactone form is more than about 95%, or more than about 90%, or more than about 85%, or more than about 80%, or more than about 75%, or more than about 70%, or more than about 65%, or more than about 60%, or more than about 55%, or more than about 50%, or more than about 25%.

In various embodiments, the modified-release formulations of the invention are designed to stabilize or prevent the interconversion of the lactone to the hydroxyacid form or other chemically defined form. In such embodiments, the stabilization of the lactone form or the prevention of the conversion to the hydroxyacid form may be achieved by varying factors such as pH, buffer concentration, and temperature of the formulation.

In various embodiments, the modified-release formulations of the present invention are designed for immediate release (e.g. upon ingestion). In various embodiments, the modified-release formulations may have sustained-release profiles, i.e. slow release of the active ingredient(s) in the body (e.g., GI tract) over an extended period of time. In various embodiments, the modified-release formulations may have a delayed-release profile, i.e. not immediately release the active ingredient(s) upon ingestion; rather, postponement of the release of the active ingredient(s) until the composition is lower in the gastrointestinal tract; for example, for release in the small intestine (e.g., one or more of duodenum, jejunum, ileum) or the large intestine (e.g., one or more of cecum, ascending, transverse, descending or sigmoid portions of the colon, and rectum). For example, a composition can be enteric coated to delay release of the active ingredient(s) until it reaches the small intestine or large intestine. In some embodiments, there is not a substantial amount of the active ingredient(s) of the present formulations in the stool.

In various embodiments, the modified-release formulation of the present invention releases (optionally as a first release) at least 60% of the statin, statin analog or derivative, or compound disclosed herein after the stomach into one or more regions of the intestine. For example, the modified-release formulation releases at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97, at least 98%, at least 99%, or 100% of the statin, statin analog or derivative, or compound disclosed herein in the intestine.

In various embodiments, the modified-release formulation releases (optionally as a first release) the statin, statin analog or derivative, or compound disclosed herein in the small intestine. In various embodiments, the modified-release formulation of the present invention releases at least 60% of the statin, statin analog or derivative, or compound disclosed herein in the small intestine. For example, the modified-release formulation releases at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% of the statin, statin analog or derivative, or compound disclosed herein in the small intestine (e.g., one or more of duodenum, jejunum, ileum, and ileocecal junction).

In other embodiments, the modified-release formulation releases (optionally as a first release) statin, statin analog or derivative, or compound disclosed herein in the large intestine. In various embodiments, the modified-release formulation of the present invention releases at least 60% of the statin, statin analog or derivative, or compound disclosed herein in the large intestine. For example, the modified-release formulation releases at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% of the statin, statin analog or derivative, or compound disclosed herein in the large intestine (e.g., one or more of cecum, ascending, transverse, descending or sigmoid portions of the colon, and rectum).

In certain embodiments, the modified-release formulation does not substantially release the statin, statin analog or derivative, or compound disclosed herein in the stomach.

In certain embodiments, the modified-release formulation releases the statin, statin analog or derivative, or compound disclosed herein at a specific pH. For example, in some embodiments, the modified-release formulation is substantially stable in an acidic environment and substantially unstable (e.g., dissolves rapidly or is physically unstable) in a near neutral to alkaline environment. In some embodiments, stability is indicative of not substantially releasing while instability is indicative of substantially releasing. For example, in some embodiments, the modified-release formulation is substantially stable at a pH of about 7.0 or less, or about 6.5 or less, or about 6.0 or less, or about 5.5 or less, or about 5.0 or less, or about 4.5 or less, or about 4.0 or less, or about 3.5 or less, or about 3.0 or less, or about 2.5 or less, or about 2.0 or less, or about 1.5 or less, or about 1.0 or less. In some embodiments, the present formulations are stable in lower pH areas and therefore do not substantially release in, for example, the stomach. In some embodiments, modified-release formulation is substantially stable at a pH of about 1 to about 4 or lower and substantially unstable at pH values that are greater. In these embodiments, the modified-release formulation does not substantially release the active ingredient(s) in the stomach. In these embodiments, the modified-release formulation substantially releases in the small intestine (e.g. one or more of the duodenum, jejunum, and ileum) and/or large intestine (e.g. one or more of the cecum, ascending colon, transverse colon, descending colon, and sigmoid colon). In some embodiments, modified-release formulation is substantially stable at a pH of about 4 to about 5 or lower and consequentially is substantially unstable at pH values that are greater and therefore does not substantially release in the stomach and/or small intestine (e.g. one or more of the duodenum, jejunum, and ileum). In these embodiments, the modified-release formulation substantially releases in the large intestine (e.g. one or more of the cecum, ascending colon, transverse colon, descending colon, and sigmoid colon). In various embodiments, the pH values recited herein may be adjusted as known in the art to account for the state of the subject, e.g. whether in a fasting or postprandial state.

In some embodiments, the modified-release formulation is substantially stable in gastric fluid and substantially unstable in intestinal fluid and, accordingly, substantially releases the active ingredient(s) in the small intestine (e.g. one or more of the duodenum, jejunum, and ileum) and/or large intestine (e.g. one or more of the cecum, ascending colon, transverse colon, descending colon, and sigmoid colon).

In some embodiments, the modified-release formulation is stable in gastric fluid or stable in acidic environments. These modified-release formulations release about 30% or less by weight of the statin, statin analog or derivative, or compound disclosed herein in the modified-release formulation in gastric fluid with a pH of about 4 to about 5 or less, or simulated gastric fluid with a pH of about 4 to about 5 or less, in about 15, or about 30, or about 45, or about 60, or about 90 minutes. Modified-release formulations of the of the invention may release from about 0% to about 30%, from about 0% to about 25%, from about 0% to about 20%, from about 0% to about 15%, from about 0% to about 10%, about 5% to about 30%, from about 5% to about 25%, from about 5% to about 20%, from about 5% to about 15%, from about 5% to about 10% by weight of the statin, statin analog or derivative, or compound disclosed herein in the modified-release formulation in gastric fluid with a pH of 4-5, or less or simulated gastric fluid with a pH of 4-5 or less, in about 15, or about 30, or about 45, or about 60, or about 90 minutes. Modified-release formulations of the invention may release about 1%, about 2%, about 3%, about 4%, about 5%, about 6%, about 7%, about 8%, about 9%, or about 10% by weight of the total statin, statin analog or derivative, or compound disclosed herein in the modified-release formulation in gastric fluid with a pH of 5 or less, or simulated gastric fluid with a pH of 5 or less, in about 15, or about 30, or about 45, or about 60, or about 90 minutes.

In some embodiments, the modified-release formulation is unstable in intestinal fluid. These modified-release formulations release about 70% or more by weight of the statin, statin analog or derivative, or compound disclosed herein in the modified-release formulation in intestinal fluid or simulated intestinal fluid in about 15, or about 30, or about 45, or about 60, or about 90 minutes. In some embodiments, the modified-release formulation is unstable in near neutral to alkaline environments. These modified-release formulations release about 70% or more by weight of the statin, statin analog or derivative, or compound disclosed herein in the modified-release formulation in intestinal fluid with a pH of about 4-5 or greater, or simulated intestinal fluid with a pH of about 4-5 or greater, in about 15, or about 30, or about 45, or about 60, or about 90 minutes. A modified-release formulation that is unstable in near neutral or alkaline environments may release 70% or more by weight of the statin, statin analog or derivative, or compound disclosed herein in the modified-release formulation in a fluid having a pH greater than about 5 (e.g., a fluid having a pH of from about 5 to about 14, from about 6 to about 14, from about 7 to about 14, from about 8 to about 14, from about 9 to about 14, from about 10 to about 14, or from about 11 to about 14) in from about 5 minutes to about 90 minutes, or from about 10 minutes to about 90 minutes, or from about 15 minutes to about 90 minutes, or from about 20 minutes to about 90 minutes, or from about 25 minutes to about 90 minutes, or from about 30 minutes to about 90 minutes, or from about 5 minutes to about 60 minutes, or from about 10 minutes to about 60 minutes, or from about 15 minutes to about 60 minutes, or from about 20 minutes to about 60 minutes, or from about 25 minutes to about 90 minutes, or from about 30 minutes to about 60 minutes.

In one embodiment, the modified-release formulation may remain essentially intact, or may be essentially insoluble, in gastric fluid. The stability of the delayed-release coating can be pH dependent. Delayed-release coatings that are pH dependent will be substantially stable in acidic environments (pH of about 5 or less), and substantially unstable in near neutral to alkaline environments (pH greater than about 5). For example, the delayed-release coating may essentially disintegrate or dissolve in near neutral to alkaline environments such as are found in the small intestine (e.g. one or more of the duodenum, jejunum, and ileum) and/or large intestine (e.g. one or more of the cecum, ascending colon, transverse colon, descending colon, and sigmoid colon).

Examples of simulated gastric fluid and simulated intestinal fluid include, but are not limited to, those disclosed in the 2005 Pharmacopeia 23NF/28USP in Test Solutions at page 2858 and/or other simulated gastric fluids and simulated intestinal fluids known to those of skill in the art, for example, simulated gastric fluid and/or intestinal fluid prepared without enzymes.

Alternatively, the stability of the modified-release formulation can be enzyme-dependent. Delayed-release coatings that are enzyme dependent will be substantially stable in fluid that does not contain a particular enzyme and substantially unstable in fluid containing the enzyme. The delayed-release coating will essentially disintegrate or dissolve in fluid containing the appropriate enzyme. Enzyme-dependent control can be brought about, for example, by using materials which release the active ingredient only on exposure to enzymes in the intestine, such as galactomannans. Also, the stability of the modified-release formulation can be dependent on enzyme stability in the presence of a microbial enzyme present in the gut flora.

In some embodiments, a dual pulse formulation is provided. In various embodiments, the present invention provides for modified-release formulations that release multiple doses of the statin, statin analog or derivative, or compound disclosed herein at different locations along the intestines, at different times, and/or at different pH. In an illustrative embodiment, the modified-release formulation comprises a first dose of the statin, statin analog or derivative, or compound disclosed herein and a second dose of the statin, statin analog or derivative, or compound disclosed herein, wherein the first dose and the second dose are released at different locations along the intestines, at different times, and/or at different pH. For example, the first dose is released at the duodenum, and the second dose is released at the ileum. In another example, the first dose is released at the jejunum, and the second dose is released at the ileum. In other embodiments, the first dose is released at a location along the small intestine (e.g., the duodenum), while the second dose is released along the large intestine (e.g., the ascending colon). In various embodiments, the modified-release formulation may release at least one dose, at least two doses, at least three doses, at least four doses, at least five doses, at least six doses, at least seven doses, or at least eight doses of the statin, statin analog or derivative, or compound disclosed herein at different locations along the intestines, at different times, and/or at different pH. Each individual dose may comprise the same statin, statin analog or derivative, or compound disclosed herein or may comprise a different statin, statin analog or derivative, or compound disclosed herein.

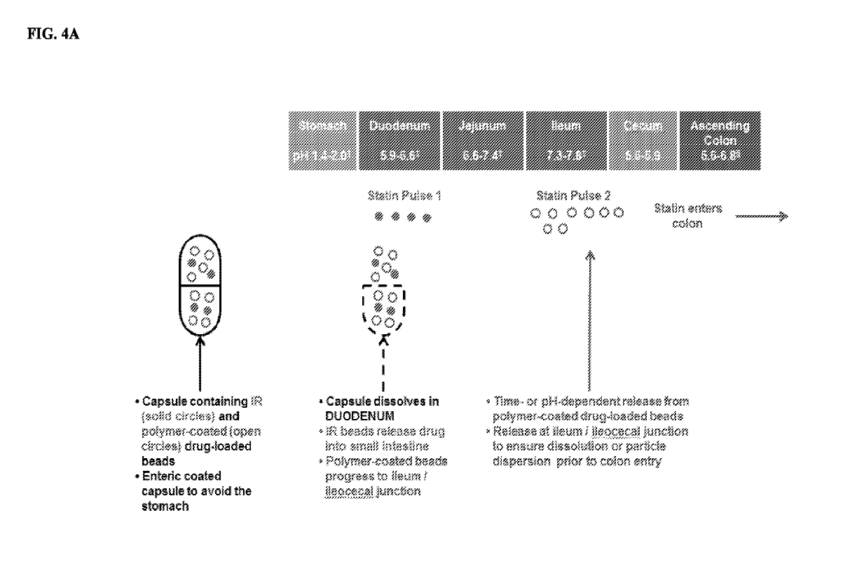

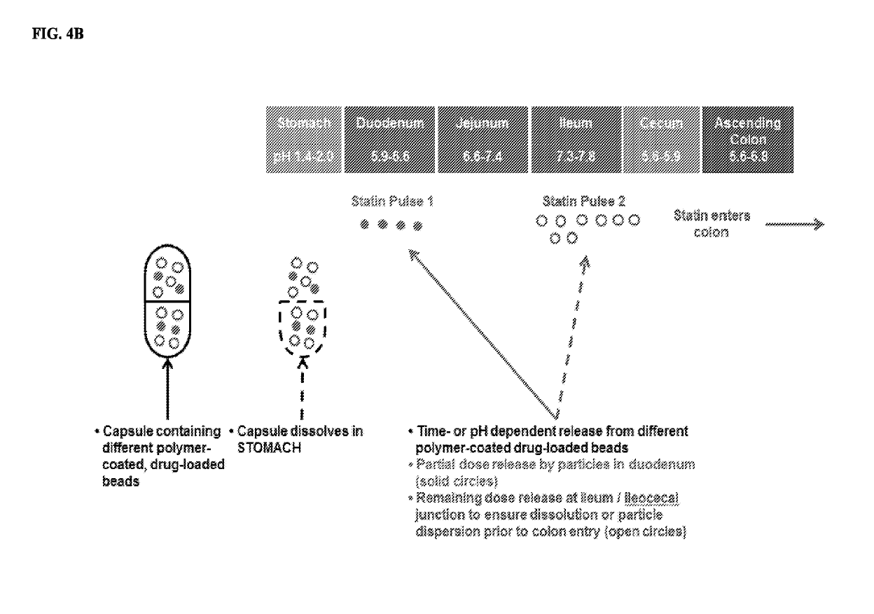

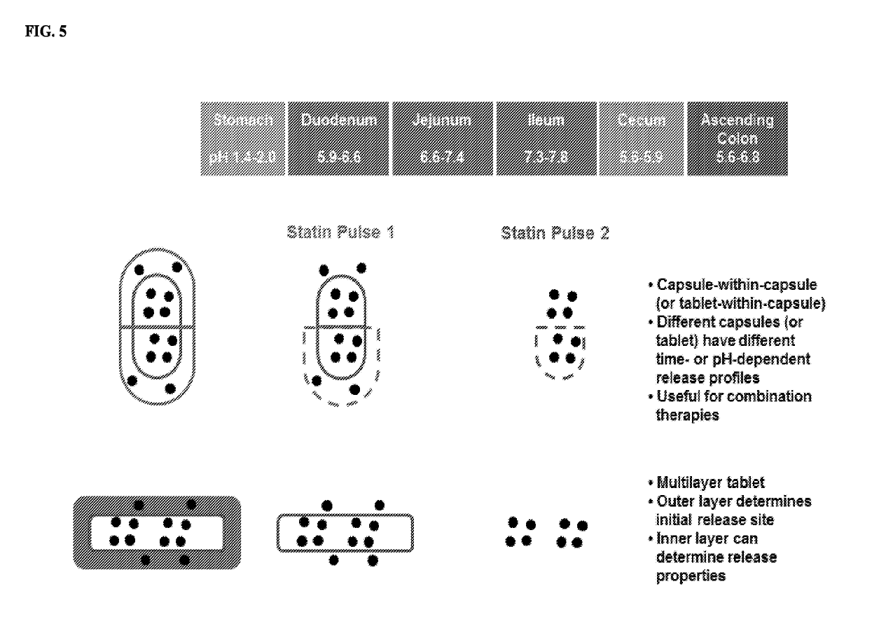

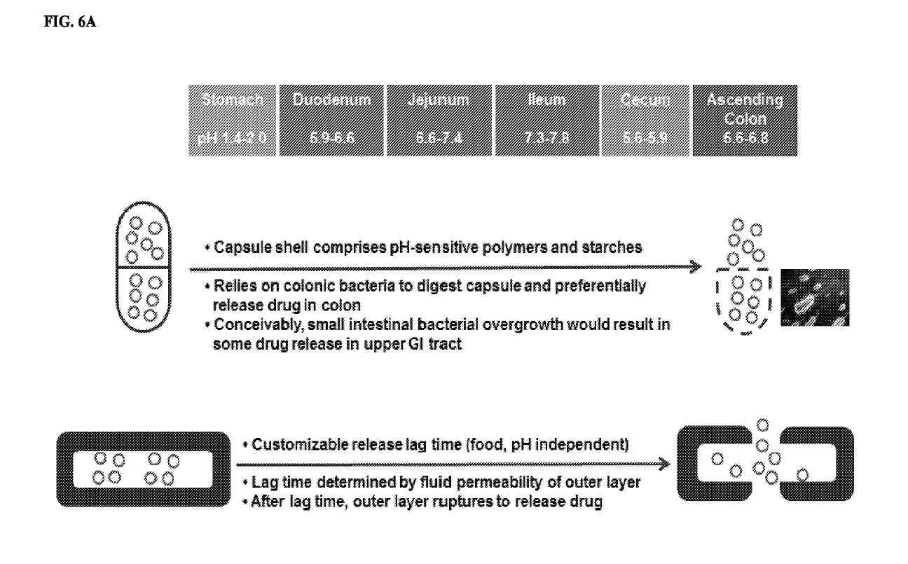

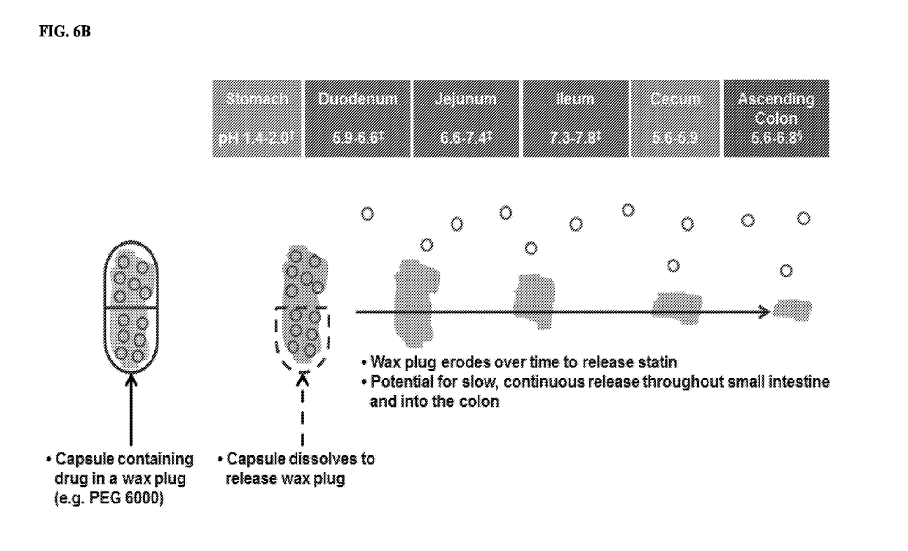

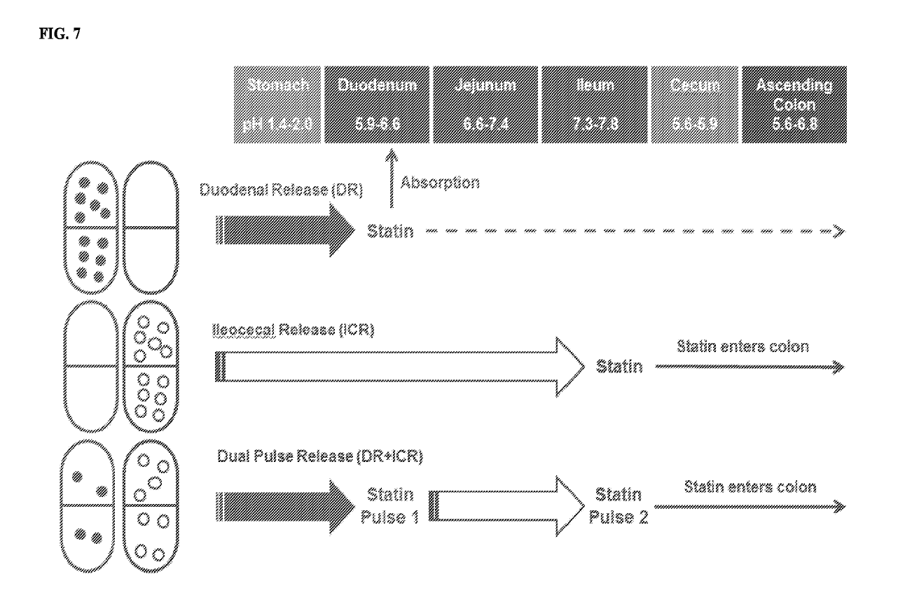

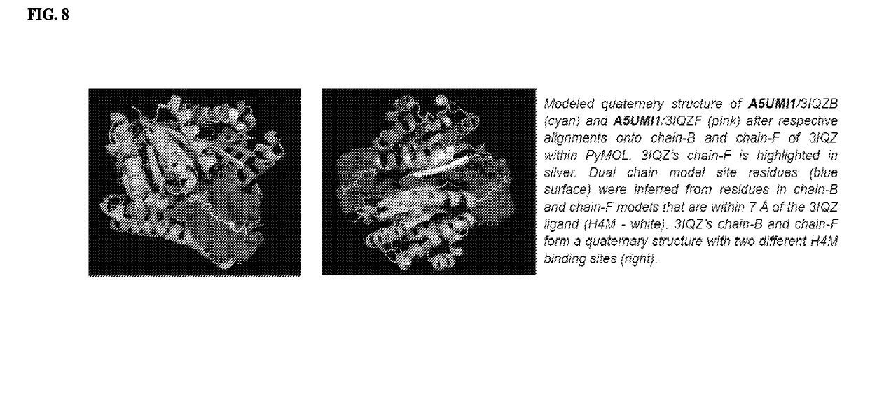

In some embodiments, the dual pulse formulation is an enteric-coated capsule comprising beads that comprise a statin, statin analog or derivative, or compound disclosed herein and optionally an additional therapeutic agent. In some embodiments, the enteric-coated capsule dissolves in a first area of GI tract to release the beads and/or a first population of beads releases in a second area of the GI tract (and that is not the same as the first area of the GI tract) and a second population of beads releases in a third area of the GI tract (and that is not the same as the first or second areas of the GI tract). In some embodiments, the dose/release ratio (e.g. how much agent is released in various locations) can be tuned as needed. In some embodiments, the enteric-coated capsule dissolves in the duodenum to release the beads and/or a first population of beads releases in the duodenum and/or a second population of beads releases in the ileocecal junction (see. e.g. FIG. 4 to FIG. 7).

Exemplary formulations comprising statins are disclosed, for example, in International Patent Application PCT/US2015/045140 and U.S. patent application Ser. No. 14/826,115, the entire contents of which are hereby incorporated by reference. Such formulations, in some embodiments, are applied to compounds described herein, inclusive of compounds of Formulas I-VI and Compounds (1)-(131),

### Dosage Forms

In various embodiments, the present invention pertains to pharmaceutical compositions comprising the statin, statin analog or derivative, or compound disclosed herein described herein and a pharmaceutically acceptable carrier or excipient. Any pharmaceutical compositions described herein can be administered to a subject as a component of a composition that comprises a pharmaceutically acceptable carrier or vehicle. Such compositions can optionally comprise a suitable amount of a pharmaceutically acceptable excipient so as to provide the form for proper administration.

In various embodiments, pharmaceutical excipients can be liquids, such as water and oils, including those of petroleum, animal, vegetable, or synthetic origin, such as peanut oil, soybean oil, mineral oil, sesame oil and the like. The pharmaceutical excipients can be, for example, saline, gum acacia, gelatin, starch paste, talc, keratin, colloidal silica, urea and the like. In addition, auxiliary, stabilizing, thickening, lubricating, and coloring agents can be used. In one embodiment, the pharmaceutically acceptable excipients are sterile when administered to a subject. Water is a useful excipient when any agent described herein is administered intravenously. Saline solutions and aqueous dextrose and glycerol solutions can also be employed as liquid excipients, specifically for injectable solutions. Suitable pharmaceutical excipients also include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. Any agent described herein, if desired, can also comprise minor amounts of wetting or emulsifying agents, or pH buffering agents. Other examples of suitable pharmaceutical excipients are described in Remington's Pharmaceutical Sciences 1447-1676 (Alfonso R. Gennaro eds., 19th ed. 1995), incorporated herein by reference.

The present invention includes the described pharmaceutical compositions (and/or additional therapeutic agents) in various formulations. Any inventive pharmaceutical composition (and/or additional therapeutic agents) described herein can take the form of solutions, suspensions, emulsion, drops, tablets, pills, pellets, capsules, capsules containing liquids, gelatin capsules, powders, sustained-release formulations, suppositories, emulsions, aerosols, sprays, suspensions, lyophilized powder, frozen suspension, desiccated powder, or any other form suitable for use. In one embodiment, the composition is in the form of a capsule. In another embodiment, the composition is in the form of a tablet. In yet another embodiment, the pharmaceutical composition is formulated in the form of a soft-gel capsule.

In a further embodiment, the pharmaceutical composition is formulated in the form of a gelatin capsule. In yet another embodiment, the pharmaceutical composition is formulated as a liquid.

The formulations comprising the inventive pharmaceutical compositions (and/or additional agents) of the present invention may conveniently be presented in unit dosage forms and may be prepared by any of the methods well known in the art of pharmacy. Such methods generally include the step of bringing the therapeutic agents into association with a carrier, which constitutes one or more accessory ingredients. Typically, the formulations are prepared by uniformly and intimately bringing the therapeutic agent into association with a liquid carrier, a finely divided solid carrier, or both, and then, if necessary, shaping the product into dosage forms of the desired formulation (e.g., wet or dry granulation, powder blends, etc., followed by tableting using conventional methods known in the art).

While certain embodiments pertain to GI-based administration, e.g. via the modified-release formulations described herein, the present invention also allows for other modes of administration such as, for example, systemic administration.

Routes of administration include, for example: oral, intradermal, intramuscular, intraperitoneal, intravenous, subcutaneous, intranasal, epidural, sublingual, intranasal, intracerebral, intravaginal, transdermal, rectally, by inhalation, or topically. Administration can be local or systemic. In some embodiments, the administering is effected orally. In another embodiment, the administration is by parenteral injection. The mode of administration can be left to the discretion of the practitioner, and depends in-part upon the site of the medical condition. In most instances, administration results in the release of any agent described herein into the bloodstream.

In one embodiment, the statin, statin analog or derivative, or compound disclosed herein is formulated in accordance with routine procedures as a composition adapted for oral administration. Compositions for oral delivery can be in the form of tablets, lozenges, aqueous or oily suspensions, granules, powders, emulsions, capsules, syrups, or elixirs, for example. Orally administered compositions can comprise one or more agents, for example, sweetening agents such as fructose, aspartame or saccharin; flavoring agents such as peppermint, oil of wintergreen, or cherry; coloring agents; and preserving agents, to provide a pharmaceutically palatable preparation. Moreover, where in tablet or pill form, the compositions can be coated to delay disintegration and absorption in the gastrointestinal tract thereby providing a sustained action over an extended period of time. Selectively permeable membranes surrounding an osmotically active driving any statin, statin analog or derivative, or compound disclosed herein are also suitable for orally administered compositions. In these latter platforms, fluid from the environment surrounding the capsule is imbibed by the driving compound, which swells to displace the agent or agent composition through an aperture. These delivery platforms can provide an essentially zero order delivery profile as opposed to the spiked profiles of immediate release formulations. A time-delay material such as glycerol monostearate or glycerol stearate can also be useful. Oral compositions can include standard excipients such as mannitol, lactose, starch, magnesium stearate, sodium saccharin, cellulose, and magnesium carbonate. In one embodiment, the excipients are of pharmaceutical grade. Suspensions, in addition to the active compounds, may contain suspending agents such as, for example, ethoxylated isostearyl alcohols, polyoxyethylene sorbitol and sorbitan esters, microcrystalline cellulose, aluminum metahydroxide, bentonite, agar-agar, tragacanth, etc., and mixtures thereof.

In various embodiments, the active therapeutic compound is mixed with at least one inert, pharmaceutically acceptable excipient or carrier such as sodium citrate, dicalcium phosphate, etc., and/or a) fillers or extenders such as starches, lactose, sucrose, glucose, mannitol, silicic acid, microcrystalline cellulose, and Bakers Special Sugar, etc., b) binders such as, for example, carboxymethylcellulose, alginates, gelatin, polyvinylpyrrolidone, sucrose, acacia, polyvinyl alcohol, polyvinylpyrrolidone, methylcellulose, hydroxypropyl cellulose, and hydroxymethyl cellulose etc., c) humectants such as glycerol, etc., d) disintegrating agents such as agar-agar, calcium carbonate, potato or tapioca starch, alginic acid, certain silicates, sodium carbonate, cross-linked polymers such as crospovidone (cross-linked polyvinylpyrrolidone), croscarmellose sodium (cross-linked sodium carboxymethylcellulose), sodium starch glycolate, etc., e) solution retarding agents such as paraffin, etc., f) absorption accelerators such as quaternary ammonium compounds, etc., g) wetting agents such as, for example, cetyl alcohol and glycerol monostearate, etc., h) absorbents such as kaolin and bentonite clay, etc., and i) lubricants such as talc, calcium stearate, magnesium stearate, solid polyethylene glycols, sodium lauryl sulfate, glyceryl behenate, etc., and mixtures of such excipients. One of skill in the art will recognize that particular excipients may have two or more functions in the oral dosage form. In the case of an oral dosage form, for example, a capsule or a tablet, the dosage form may also comprise buffering agents.

The solid oral dosage forms can be prepared by any conventional method known in the art, for example granulation (e.g., wet or dry granulation) of the active compound (e.g., statins, statin analogs or derivatives, or compounds disclosed herein) with one or more suitable excipients. Alternatively, the active compound can be layered onto an inert core (e.g., a nonpareil/sugar sphere or silica sphere) using conventional methods such as fluidized bed or pan coating, or extruded and spheronized using methods known in the art, into active compound-containing beads. Such beads can then be incorporated into tablets or capsules using conventional methods.

Liquid dosage forms for oral administration include pharmaceutically acceptable emulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethyl formamide, oils (in particular, cottonseed, groundnut, corn, germ, olive, castor, and sesame oils), glycerol, tetrahydrofurfuryl alcohol, polyethylene glycols and fatty acid esters of sorbitan, etc., and mixtures thereof.

Besides inert diluents, the oral compositions can also include adjuvants such as sweetening, flavoring, and perfuming agents.

Suspensions, in addition to the active compounds, may contain suspending agents such as, for example, ethoxylated isostearyl alcohols, polyoxyethylene sorbitol and sorbitan esters, microcrystalline cellulose, aluminum metahydroxide, bentonite, agar-agar, tragacanth, etc., and mixtures thereof. Dosage forms suitable for parenteral administration (e.g. intravenous, intramuscular, intraperitoneal, subcutaneous and intra-articular injection and infusion) include, for example, solutions, suspensions, dispersions, emulsions, and the like. They may also be manufactured in the form of sterile solid compositions (e.g. lyophilized composition), which can be dissolved or suspended in sterile injectable medium immediately before use. They may contain, for example, suspending or dispersing agents known in the art. Formulation components suitable for parenteral administration include a sterile diluent such as water for injection, saline solution, fixed oils, polyethylene glycols, glycerine, propylene glycol or other synthetic solvents; antibacterial agents such as benzyl alcohol or methyl paraben; antioxidants such as ascorbic acid or sodium bisulfite; chelating agents such as EDTA; buffers such as acetates, citrates or phosphates; and agents for the adjustment of tonicity such as sodium chloride or dextrose.

For intravenous administration, suitable carriers include physiological saline, bacteriostatic water, Cremophor ELTM (BASF, Parsippany, N.J.) or phosphate buffered saline (PBS). The carrier should be stable under the conditions of manufacture and storage, and should be preserved against microorganisms. The carrier can be a solvent or dispersion medium containing, for example, water, ethanol, polyol (for example, glycerol, propylene glycol, and liquid polyetheylene glycol), and suitable mixtures thereof.

The compositions provided herein, alone or in combination with other suitable components, can be made into aerosol formulations (i.e., “nebulized”) to be administered via inhalation. Aerosol formulations can be placed into pressurized acceptable propellants, such as dichlorodifluoromethane, propane, nitrogen, and the like.

In various embodiments, the formulations of the invention are designed to stabilize or prevent the interconversion of the statin lactone to the hydroxyacid form or other chemically defined form. In such embodiments, the stabilization of the lactone form or the prevention of the conversion to the hydroxyacid form may be achieved by varying factors such as pH, buffer concentration, and temperature of the formulation.

Any inventive pharmaceutical compositions (and/or additional agents) described herein can be administered by controlled-release or sustained-release means or by delivery devices that are well known to those of ordinary skill in the art. Examples include, but are not limited to, those described in U.S. Pat. Nos. 3,845,770; 3,916,899; 3,536,809; 3,598,123; 4,008,719; 5,674,533; 5,059,595; 5,591,767; 5,120,548; 5,073,543; 5,639,476; 5,354,556; and 5,733,556, each of which is incorporated herein by reference in its entirety. Such dosage forms can be useful for providing controlled- or sustained-release of one or more active ingredients using, for example, hydropropyl cellulose, hydropropylmethyl cellulose, polyvinylpyrrolidone, other polymer matrices, gels, permeable membranes, osmotic systems, multilayer coatings, microparticles, liposomes, microspheres, or a combination thereof to provide the desired release profile in varying proportions. Suitable controlled- or sustained-release formulations known to those skilled in the art, including those described herein, can be readily selected for use with the active ingredients of the agents described herein. The invention thus provides single unit dosage forms suitable for oral administration such as, but not limited to, tablets, capsules, gelcaps, and caplets that are adapted for controlled- or sustained-release.

Controlled- or sustained-release of an active ingredient can be stimulated by various conditions, including but not limited to, changes in pH, changes in temperature, stimulation by an appropriate wavelength of light, concentration or availability of enzymes, concentration or availability of water, or other physiological conditions or compounds.

In another embodiment, a controlled-release system can be placed in proximity of the target area to be treated, thus requiring only a fraction of the systemic dose (see, e.g., Goodson, in Medical Applications of Controlled Release, supra, vol. 2, pp. 115-138 (1984)). Other controlled-release systems discussed in the review by Langer, 1990, Science 249:1527-1533) may be used.

Pharmaceutical formulations (including the modified-release formulation of the invention) preferably are sterile. Sterilization can be accomplished, for example, by filtration through sterile filtration membranes. Where the composition is lyophilizcd, filter sterilization can be conducted prior to or following lyophilization and reconstitution.

Where necessary, the inventive pharmaceutical compositions (and/or additional agents) can also include a solubilizing agent. Also, the agents can be delivered with a suitable vehicle or delivery device as known in the art. Combination therapies outlined herein can be co-delivered in a single delivery vehicle or delivery device.

The formulation (including the modified-release formulation of the invention) can additionally include a surface active agent. Surface active agents suitable for use in the present invention include, but are not limited to, any pharmaceutically acceptable, non-toxic surfactant. Classes of surfactants suitable for use in the compositions of the invention include, but are not limited to polyethoxylated fatty acids, PEG-fatty acid diesters, PEG-fatty acid mono- and di-ester mixtures, polyethylene glycol glycerol fatty acid esters, alcohol-oil transesterification products, polyglycerized fatty acids, propylene glycol fatty acid esters, mixtures of propylene glycol esters-glycerol esters, mono- and diglycerides, sterol and sterol derivatives, polyethylene glycol sorbitan fatty acid esters, polyethylene glycol alkyl ethers, sugar esters, polyethylene glycol alkyl phenols, polyoxyethylene-olyoxypropylene block copolymers, sorbitan fatty acid esters, lower alcohol fatty acid esters, ionic surfactants, and mixtures thereof. In some embodiments, compositions of the invention may comprise one or more surfactants including, but not limited to, sodium lauryl sulfate, polysorbate 20, polysorbate 40, polysorbate 60, polysorbate 80, and triethyl citrate.

The formulation (including the modified-release formulation of the invention) can also contain pharmaceutically acceptable plasticizers to obtain the desired mechanical properties such as flexibility and hardness. Such plasticizers include, but are not limited to, triacetin, citric acid esters, phthalic acid esters, dibutyl sebacate, cetyl alcohol, polyethylene glycols, polysorbates or other plasticizers.

The formulation (including the modified-release formulation of the invention) can also include one or more application solvents. Some of the more common solvents that can be used to apply, for example, a delayed-release coating composition include isopropyl alcohol, acetone, methylene chloride and the like.

The formulation (including the modified-release formulation of the invention) can also include one or more alkaline materials. Alkaline material suitable for use in compositions of the invention include, but are not limited to, sodium, potassium, calcium, magnesium and aluminum salts of acids such as phosphoric acid, carbonic acid, citric acid and other aluminum/magnesium compounds. In addition the alkaline material may be selected from antacid materials such as aluminum hydroxides, calcium hydroxides, magnesium hydroxides and magnesium oxide.

In various embodiments, the modified-release formulation of the present invention may utilize one or more modified-release coatings such as delayed-release coatings to provide for effective, delayed yet substantial delivery of the statin, statin analog or derivative, and compound disclosed herein to the GI tract together with, optionally, other therapeutic agents.

In one embodiment, the delayed-release coating includes an enteric agent that is substantially stable in acidic environments and substantially unstable in near neutral to alkaline environments. In an embodiment, the delayed-release coating contains an enteric agent that is substantially stable in gastric fluid. The enteric agent can be selected from, for example, solutions or dispersions of methacrylic acid copolymers, cellulose acetate phthalate, hydroxypropylmethyl cellulose phthalate, polyvinyl acetate phthalate, carboxymethylethylcellulose, and EUDRAGIT®-type polymer (poly(methacrylic acid, methylmethacrylate), hydroxypropyl methylcellulose acetate succinate, cellulose acetate trimellitate, shellac or other suitable enteric coating polymers. The EUDRAGIT®-type polymer include, for example, EUDRAGIT® FS 30D, L 30 D-55, L 100-55, L 100, L 12,5, L 12,5 P, RL 30 D, RL PO, RL 100, RL 12,5, RS 30 D, RS PO, RS 100, RS 12,5, NE 30 D, NE 40 D, NM 30 D, S 100, S 12,5, and S 12,5 P. In some embodiments, one or more of EUDRAGIT® FS 30D, L 30 D-55, L 100-55, L 100, L 12,5, L 12,5 P RL 30 D, RL PO, RL 100, RL 12,5, RS 30 D, RS PO, RS 100, RS 12,5, NE 30 D, NE 40 D, NM 30 D, S 100, S 12,5 and S 12,5 P is used. The enteric agent may be a combination of the foregoing solutions or dispersions.

In another embodiment, the delayed-release coating may degrade as a function of time when in aqueous solution without regard to the pH and/or presence of enzymes in the solution. Such a coating may comprise a water insoluble polymer. Its solubility in aqueous solution is therefore independent of the pH. The term “pH independent” as used herein means that the water permeability of the polymer and its ability to release pharmaceutical ingredients is not a function of pH and/or is only very slightly dependent on pH. Such coatings may be used to prepare, for example, sustained release formulations. Suitable water insoluble polymers include pharmaceutically acceptable non-toxic polymers that are substantially insoluble in aqueous media, e.g., water, independent of the pH of the solution. Suitable polymers include, but are not limited to, cellulose ethers, cellulose esters, or cellulose ether-esters, i.e., a cellulose derivative in which some of the hydroxy groups on the cellulose skeleton are substituted with alkyl groups and some are modified with alkanoyl groups. Examples include ethyl cellulose, acetyl cellulose, nitrocellulose, and the like. Other examples of insoluble polymers include, but are not limited to, lacquer, and acrylic and/or methacrylic ester polymers, polymers or copolymers of acrylate or methacrylate having a low quaternary ammonium content, or mixture thereof and the like. Other examples of insoluble polymers include EUDRAGIT RS®, EUDRAGIT RL®, and EUDRAGIT NE®. Insoluble polymers useful in the present invention include polyvinyl esters, polyvinyl acetals, polyacrylic acid esters, butadiene styrene copolymers, and the like. In one embodiment, colonic delivery is achieved by use of a slowly-eroding wax plug (e.g., various PEGS, including for example, PEG6000).

In a further embodiment, the delayed-release coating may be degraded by a microbial enzyme present in the gut flora. In one embodiment, the delayed-release coating may be degraded by a bacteria present in the small intestine. In another embodiment, the delayed-release coating may be degraded by a bacteria present in the large intestine.

In an illustrative embodiment, the statin, statin analog or derivative, or compound disclosed herein is a prodrug which may be converted by an enzyme produced by the targeted organism to a pharmaceutically active form, for example, by a methanogenesis-related F₄₂₀-dependent enzyme. Accordingly, in some embodiments, the statin, statin analog or derivative, or compound disclosed herein is activated locally by the targeted F₄₂₀-dependent enzyme or another enzyme produced by the targeted organism. For example, the enzyme produced by the organism, whether F₄₂₀-dependent or not, may cleave off a moiety from the statin, statin analog or derivative, or compound disclosed herein prodrug thus activating the statin, statin analog or derivative, or compound disclosed herein locally.

The present invention provides for modified-release formulations that release multiple doses of the statin, statin analog or derivative, or compound disclosed herein along the gastrointestinal tract. The overall release profile of such a formulation may be adjusted by utilizing, for example, multiple particle types or multiple layers. In one embodiment, the first dose of the statin, statin analog or derivative, or compound disclosed herein may be formulated for release in, for example, the duodenum, whereas the second dose is formulated for delayed release in, for example, the ileum. In another embodiment, the first dose of the statin, statin analog or derivative, or compound disclosed herein may be formulated for release in, for example, the small intestines, whereas the second dose is formulated for delayed release in, for example, the large intestines. Alternatively, multiple doses are released at different locations alone the intestine.

In one embodiment, one or more doses of the statin, statin analog or derivative, or compound disclosed herein may be encapsulated in a core particle, for example, in the form of a microbead. For example, the first dose of the statin, statin analog or derivative, or compound disclosed herein may be encapsulated in a core particle coated with a modified-release coating designed for release at a first location along the intestinal tract, and the second dose of the statin, statin analog or derivative, or compound disclosed herein may be encapsulated in a core particle coated with a modified-release coating designed for release at a second location along the intestinal tract. The formulation may comprise a plurality of such modified-release particles. For example, the formulation is in the form of capsules comprising multiple microbeads. In such an embodiment, a combination of microbeads may be utilized in which each microbead is designed to release at a specific time point or location. In an alternative embodiment, the formulation is formulated as a capsule within a capsule, with each capsule having different time- or pH-dependent release properties.

In another embodiment, one or more doses of the statin, statin analog or derivative, or compound disclosed herein may be encapsulated in a layer. For example, the first dose of the statin, statin analog or derivative, or compound disclosed herein may be encapsulated in a layer coated with a modified-release coating designed for release at a first location along the intestinal tract, and the second dose of the statin, statin analog or derivative, or compound disclosed herein may be encapsulated in a layer coated with a modified-release coating designed for release at a second location along the intestinal tract. The formulation may comprise a plurality of such modified-release layers. For example, the formulation is in the form of multi-layered tablet or a multi-layered capsule. Each layer may have different time- or pH-dependent release properties.

In the above embodiments, the coated particles or layers with the delayed-release coating may be further covered with an overcoat layer. The overcoat layer can be applied as described for the other coating compositions. The overcoat materials are pharmaceutically acceptable compounds such as sugar, polyethylene glycol, polyvinylpyrrolidone, polyvinyl alcohol, polyvinyl acetate, hydroxypropyl cellulose, methylcellulose, ethylcellulose, hydroxypropyl methylcellulose, carboxymethylcellulose sodium and others, used alone or in mixtures. The overcoat materials can prevent potential agglomeration of particles coated with the delayed-release coating, protect the delayed-release coating from cracking during the compaction process or enhance the tableting process.

Furthermore, in various embodiments, the agents described herein may be in the form of a pharmaceutically acceptable salt, namely those salts which are suitable for use in contact with the tissues of humans and other animals without undue toxicity, irritation, allergic response and the like, and are commensurate with a reasonable benefit/risk ratio. Pharmaceutically acceptable salts are well known in the art. The salts can be prepared in situ during the final isolation and purification of the therapeutic agents, or separately by reacting the free base function with a suitable acid or a free acid functionality with an appropriate alkaline moiety. Representative acid addition salts include acetate, adipate, alginate, ascorbate, aspartate, benzenesulfonate, benzoate, bisulfate, borate, butyrate, camphorate, camphersulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxyethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts, and the like. Representative alkali or alkaline earth metal salts include sodium, lithium, potassium, calcium, magnesium, and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like.

The therapeutic agents or their pharmaceutically acceptable salts which are used in accordance with the present invention may exhibit stereoisomerism by virtue of the presence of one or more asymmetric or chiral centers in the compounds. The present invention contemplates the various stereoisomers and mixtures thereof. Desired enantiomers can be obtained by chiral synthesis from commercially available chiral starting materials by methods well known in the art, or may be obtained from mixtures of the enantiomers by resolution using known techniques.

### Administration and Dosage

It will be appreciated that the actual dose of the statin, statin analog or derivative, and compound disclosed herein to be administered according to the present invention will vary according to the particular compound, the particular dosage form, and the mode of administration. Many factors that may modify the action of the statin, statin analog or derivative, and compound disclosed herein (e.g., body weight, gender, diet, time of administration, route of administration, rate of excretion, condition of the subject, drug combinations, genetic disposition and reaction sensitivities) can be taken into account by those skilled in the art. Administration can be carried out continuously or in one or more discrete doses within the maximum tolerated dose. Optimal administration rates for a given set of conditions can be ascertained by those skilled in the art using conventional dosage administration tests.

Individual doses of the statin, statin analog or derivative, and compound disclosed herein can be administered in unit dosage forms (e.g., tablets or capsules) containing, for example, from about 0.01 mg to about 100 mg, from about 0.1 mg to about 100 mg, from about 0.1 mg to about 90 mg, from about 0.1 mg to about 80 mg, from about 0.1 mg to about 70 mg, from about 0.1 mg to about 60 mg, from about 0.1 mg to about 50 mg, from about 0.1 mg to about 40 mg active ingredient, from about 0.1 mg to about 30 mg, from about 0.1 mg to about 20 mg, from about 0.1 mg to about 10 mg, from about 0.1 mg to about 5 mg, from about 0.1 mg to about 3 mg, from about 0.1 mg to about 1 mg per unit dosage form, or from about 5 mg to about 80 mg per unit dosage form. For example, a unit dosage form can be about 0.01 mg, about 0.02 mg, about 0.03 mg, about 0.04 mg, about 0.05 mg, about 0.06 mg, about 0.07 mg, about 0.08 mg, about 0.09 mg, about 0.1 mg, about 0.2 mg, about 0.3 mg, about 0.4 mg, about 0.5 mg, about 0.6 mg, about 0.7 mg, about 0.8 mg, about 0.9 mg, about 1 mg, about 2 mg, about 3 mg, about 4 mg, about 5 mg, about 6 mg, about 7 mg, about 8 mg, about 9 mg about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, or about 100 mg, inclusive of all values and ranges therebetween.

In one embodiment, the statin, statin analog or derivative, or compound disclosed herein is administered at an amount of from about 0.01 mg to about 100 mg daily, an amount of from about 0.1 mg to about 100 mg daily, from about 0.1 mg to about 95 mg daily, from about 0.1 mg to about 90 mg daily, from about 0.1 mg to about 85 mg daily, from about 0.1 mg to about 80 mg daily, from about 0.1 mg to about 75 mg daily, from about 0.1 mg to about 70 mg daily, from about 0.1 mg to about 65 mg daily, from about 0.1 mg to about 60 mg daily, from about 0.1 mg to about 55 mg daily, from about 0.1 mg to about 50 mg daily, from about 0.1 mg to about 45 mg daily, from about 0.1 mg to about 40 mg daily, from about 0.1 mg to about 35 mg daily, from about 0.1 mg to about 30 mg daily, from about 0.1 mg to about 25 mg daily, from about 0.1 mg to about 20 mg daily, from about 0.1 mg to about 15 mg daily, from about 0.1 mg to about 10 mg daily, from about 0.1 mg to about 5 mg daily, from about 0.1 mg to about 3 mg daily, from about 0.1 mg to about 1 mg daily, or from about 5 mg to about 80 mg daily. In various embodiments, the statin, statin analog or derivative, or compound disclosed herein is administered at a daily dose of about 0.01 mg, about 0.02 mg, about 0.03 mg, about 0.04 mg, about 0.05 mg, about 0.06 mg, about 0.07 mg, about 0.08 mg, about 0.09 mg, about 0.1 mg, about 0.2 mg, about 0.3 mg, about 0.4 mg, about 0.5 mg, about 0.6 mg, about 0.7 mg, about 0.8 mg, about 0.9 mg, about 1 mg, about 2 mg, about 3 mg, about 4 mg, about 5 mg, about 6 mg, about 7 mg, about 8 mg, about 9 mg about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, or about 100 mg, inclusive of all values and ranges therebetween.

In some embodiments, a suitable dosage of the statin, statin analog or derivative, or compound disclosed herein is in a range of about 0.01 mg/kg to about 10 mg/kg of body weight of the subject, for example, about 0.01 mg/kg, about 0.02 mg/kg, about 0.03 mg/kg, about 0.04 mg/kg, about 0.05 mg/kg, about 0.06 mg/kg, about 0.07 mg/kg, about 0.08 mg/kg, about 0.09 mg/kg, about 0.1 mg/kg, about 0.2 mg/kg, about 0.3 mg/kg, about 0.4 mg/kg, about 0.5 mg/kg, about 0.6 mg/kg, about 0.7 mg/kg, about 0.8 mg/kg, about 0.9 mg/kg, about 1 mg/kg, about 1.1 mg/kg, about 1.2 mg/kg, about 1.3 mg/kg, about 1.4 mg/kg, about 1.5 mg/kg, about 1.6 mg/kg, about 1.7 mg/kg, about 1.8 mg/kg, 1.9 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg body weight, inclusive of all values and ranges therebetween. In other embodiments, a suitable dosage of the statin, statin analog or derivative, or compound disclosed herein is in a range of about 0.01 mg/kg to about 10 mg/kg of body weight, in a range of about 0.01 mg/kg to about 9 mg/kg of body weight, in a range of about 0.01 mg/kg to about 8 mg/kg of body weight, in a range of about 0.01 mg/kg to about 7 mg/kg of body weight, in a range of 0.01 mg/kg to about 6 mg/kg of body weight, in a range of about 0.05 mg/kg to about 5 mg/kg of body weight, in a range of about 0.05 mg/kg to about 4 mg/kg of body weight, in a range of about 0.05 mg/kg to about 3 mg/kg of body weight, in a range of about 0.05 mg/kg to about 2 mg/kg of body weight, in a range of about 0.05 mg/kg to about 1.5 mg/kg of body weight, or in a range of about 0.05 mg/kg to about 1 mg/kg of body weight.

In accordance with certain embodiments of the invention, the statin, statin analog or derivative, and compound disclosed herein may be administered, for example, more than once daily (e.g., about two times, about three times, about four times, about five times, about six times, about seven times, about eight times, about nine times, or about ten times daily), about once per day, about every other day, about every third day, about once a week, about once every two weeks, about once every month, about once every two months, about once every three months, about once every six months, or about once every year.

In various embodiments, the statin, statin analog or derivative, and compound or formulation disclosed herein may be administered in a patient that is fasting. In various embodiments, the statin, statin analog or derivative, and compound or formulation disclosed herein may be administered in a patient with a meal. In various embodiments, the statin, statin analog or derivative, and compound or formulation disclosed herein may be administered in a patient that is postprandial. In various embodiments, patient is on an elemental diet. A comestible total enteral nutrition (TEN) formulation, which is also called an “elemental diet” are commercially available, for example, VIVONEX T.E.N. (Nestle) and its variants, or the like. A useful total enteral nutrition formulation satisfies all the subject's nutritional requirements, containing free amino acids, carbohydrates, lipids, and all essential vitamins and minerals, but is in a form that is readily absorbable in the upper gastrointestinal tract, thus depriving or “starving” the methanogen syntrophic microorganism of nutrients or at least some of the nutrients they use for proliferating. Thus, methanogen syntrophic microorganism growth is inhibited.

### Additional Agents and Combination Therapy or Co-Formulation

Administration of the statin, statin analog or derivative, and compound or formulation disclosed herein may be combined with additional therapeutic agents. Co-administration of the additional therapeutic agent and the present formulations may be simultaneous or sequential. Further the present formulations may comprise an additional therapeutic agent (e.g. via co-formulation).

In some embodiments, the statin, statin analog or derivative, and compound or formulation as described herein are administered in combination with an additional therapeutic agent. In an embodiment, the additional therapeutic agent and the statin, statin analog or derivative, or compound disclosed herein, are combined into a single formulation. In some embodiments, the methods of treatment and/or prevention comprise administering the statin, statin analog or derivative, and compound or formulation disclosed herein to a subject that is undergoing treatment with an additional therapeutic agent.

In one embodiment, the additional therapeutic agent and the statin, statin analog or derivative, or compound disclosed herein is administered to a subject simultaneously. The term “simultaneously” as used herein, means that the additional agent and the statin, statin analog or derivative, or compound disclosed herein are administered with a time separation of no more than about 60 minutes, such as no more than about 30 minutes, no more than about 20 minutes, no more than about 10 minutes, no more than about 5 minutes, or no more than about 1 minute. Administration of the additional agent and the statin, statin analog or derivative, or compound disclosed herein can be by simultaneous administration of a single formulation (e.g., a formulation comprising the additional therapeutic agent and the statin, statin analog or derivative, or compound disclosed herein) or of separate formulations (e.g., a first formulation including the additional therapeutic agent and a second formulation including the statin, statin analog or derivative, or compound disclosed herein).

Co-administration does not require the therapeutic agents to be administered simultaneously, if the timing of their administration is such that the pharmacological activities of the additional agent and the statin, statin analog or derivative, or compound disclosed herein overlap in time, thereby exerting a combined therapeutic effect. For example, the additional agent and the statin, statin analog or derivative, or compound disclosed herein can be administered sequentially. The term “sequentially” as used herein means that the additional therapeutic agent and the statin, statin analog or derivative, or compound disclosed herein are administered with a time separation of more than about 60 minutes. For example, the time between the sequential administration of the additional therapeutic agent and the statin, statin analog or derivative, or compound disclosed herein can be more than about 60 minutes, more than about 2 hours, more than about 5 hours, more than about 10 hours, more than about 1 day, more than about 2 days, more than about 3 days, or more than about 1 week apart. The optimal administration times will depend on the rates of metabolism, excretion, and/or the pharmacodynamic activity of the additional agent and the statin, statin analog or derivative, and compound disclosed herein being administered. Either the additional therapeutic agent or the statin, statin analog or derivative, or compound disclosed herein may be administered first.

In a further embodiment, the additional therapeutic agent and the statin, statin analog or derivative, or compound disclosed herein are administered to a subject simultaneously but the release of the additional therapeutic agent and the statin, statin analog or derivative, or compound disclosed herein from their respective dosage forms (or single unit dosage form if co-formulated) in the GI tract occurs sequentially.

Co-administration also does not require the therapeutic agents to be administered to the subject by the same route of administration. Rather, each therapeutic agent can be administered by any appropriate route, for example, parenterally or non-parenterally.

In some embodiments, the formulation may further include agent which prevents or reduces lactone ring-opening, such as an esterase inhibitor (e.g. grapefruit juice or components naringenin, kaempferol) and/or a paraoxonase inhibitor (e.g. PON1 or PON3 inhibitor). In some embodiments, the esterase inhibitor and/or a paraoxonase inhibitor is one or more of amiodarone, anastrozole, azithromyzcin, cannabinoids, cimetidine, clarithromycin, clotrimazole, cyclosporine, danazol, delavirdine, dexamethasone, diethyldithiocarbamate, diltiazem, dirithyromycin, disulfiram, entacapone, erythromycin, ethinyl estradiol, fluconazole, fluoxetine, fluvoaxamine, gestodene, grapefruit juice, indinavir, isoniazid, ketoconazole, metronidazole, mibefradil, miconazole, nefazodone, nelfinavir, nevirapine, norfloxacin, norfluoxetine, omeprazole, oxiconazole, paroxetine, propoxyphene, quinidine, quinine, quinupristine and dalfopristin, ranitidine, ritonavir, saquinavir, sertindole, sertraline, troglitazone, troleandomycin, valproic acid and/or a lactam agent selected from oxindole, isatin, δ-valerolactam, ε-caprolactam, 2-hydroxyquinoline, and 3,4-dihydro-2(1H)-quinoline and N-bromo-ε-caprolactam.

In embodiments, the statin, statin analog or derivative, or compound or formulation as described herein is administered in combination with an inhibitor of the organic anion transporting polypeptide (OATP) transporter. In an embodiment, the OATP inhibitor and the statin, statin analog or derivative, or compound disclosed herein are combined into a single formulation. Without wishing to be bound by theory, it is believed that inclusion of the OATP inhibitor minimizes absorption of the statin, statin analog or derivative, or compound disclosed herein from the intestine and/or reduces the enterohepatic recirculation of the statin, statin analog or derivative, or compound disclosed herein, thereby maximizing retention of the statin, statin analog or derivative, or compound disclosed herein in the intestine and minimizing any potential systemic side effects of the statin, statin analog or derivative, or compound disclosed herein. Illustrative OATP inhibitors include, but are not limited to, grapefruit juice or grapefruit juice constituents such as naringin and hesperidin, orange juice and orange juice constituents, apple juice and apple juice constituents, and green tea and green tea extracts such as epicatechin gallate (ECG), epigallocatechin gallate (EGCG). In an embodiment, the OATP inhibitor is released in the intestine prior to release of the statin, statin analog or derivative, or compound disclosed herein.

In one embodiment, the additional therapeutic agent is an agent that inhibits methanogenesis. Exemplary methanogenesis inhibitors include, but are not limited to, structural analogs of coenzyme M such as 2-bromoethanesulfonate (BES), 2-chloroethanesulfonate (CES), 2-mercaptoethanesulfonate (MES), and lumazine, medium or long chain fatty acids, such as lauric acid and hexadecatrienoic acid, and nitrocompounds such as nitrate, nitrite, nitroethane, and 2-nitropropanol, phosphate, sulfate, alkoxylates of mono- and poly-valent alcohols, red yeast rice, vitamin B 10 derivatives, and ethanesulfonates. Additional agents that inhibit methanogenesis are disclosed, for example, in U.S. Patent Publication Nos. 20150208691, 20120219527, and 20140251900 and Liu et al., Appl Microbiol Biotechnol. (2011), 89(5): 1333-40, the entire contents of which are hereby incorporated by reference.

In one embodiment, the additional therapeutic agent is a prokinetic agent that facilitates movement of a mass through the intestinal tract. Illustrative prokinetic agents include, but are not limited to, prucalopride (e.g. RESOLOR), metoclopramide, cisapride, domperidone, or a macrolide antibiotic such as erythromycin. In another embodiment, the additional therapeutic agent is a natural product such as peppermint oil, which alleviates abdominal pain.

The present invention also contemplates the use of additional therapeutic agent that are useful for treating constipation such as, for example, laxatives, guanylate cyclase C agonist (e.g., linaclotide), a serotonin agonist (e.g., prucalopride, tegaserod), a chloride channel agonist (e.g., lubiprostone), and combinations thereof.

In some embodiments, the additional therapeutic agent is an agent useful for treating IBS (including IBS-C). In some embodiments, the additional therapeutic agent is a selective chloride channel activator, including, for example, molecules derived from prostaglandins such as lubiprostone (e.g. AMITIZA) and those compounds described in U.S. Pat. Nos. 5,284,858, 6,414,016 and 6,583,174, the contents of which are hereby incorporated by reference in their entireties. In some embodiments, the additional therapeutic agent is an agent, including a peptide agent, that increases the secretion of chloride and/or water in the intestines and/or soften stools and/or stimulate bowel movements, such as, for example, linaclotide (e.g. LINZESS) and those compounds described in U.S. Pat. No. 7,304,036, the contents of which are hereby incorporated by reference in their entirety. In some embodiments, the additional therapeutic agent is an agent that relaxes the colon and/or slows the movement of waste through the lower bowel. In some embodiments the additional therapeutic agent is a 5-HT₃ antagonist, including, but not limited to, alosetron (e.g. LOTRONEX).

In some embodiments, the additional therapeutic agent is a small molecule that acts as a peripherally selective κ-opioid agonist, such as, for example, EMD-61753 ((N-methyl-N-[(1S)-1-phenyl-2-((3S)-3-hydroxypyrrolidin-1-yl)-ethyl]-2,2-diphenyl-acetamide hydrochloride, ASMADOLINE) and those compounds described in U.S. Pat. No. 6,344,566, the contents of which are hereby incorporated by reference in their entirety. In some embodiments, the additional therapeutic agent is a cholecystokinin antagonist, e.g. one selective for the CCK_{A} subtype and/or inhibits gastrointestinal motility and reduces gastric secretions, such as, for example, Dexloxiglumide ((4R)-4-[(3,4-dichlorobenzoyl)amino]-5-(3-methoxypropylpentylamino)-5-oxopentanoic acid) and those compounds described in U.S. Pat. No. 5,602,179, the contents of which are hereby incorporated by reference in their entirety. In some embodiments, the additional therapeutic agent is tapentadol (1R,2R)-3-(3-dimethylamino-1-ethyl-2-methyl-propyl)-phenol), as described in US Patent Publication No. 2013/0116334, the contents of which are hereby incorporated by reference in their entirety In some embodiments, the additional therapeutic agent is a laxative, including but not limited to osmotic laxatives (such as, for example, magnesium carbonate, magnesium hydroxide (e.g. Milk of Magnesia), magnesium oxide, magnesium peroxide, magnesium sulfate, lactulose, lactitol, sodium sulfate, pentacrythritol, macrogol, mannitol, sodium phosphate, sorbitol, magnesium citrate, sodium tartrate, laminarid, and polyethylene glycol (e.g., macrogol-containing products, such as MOVICOL and polyethylene glycol 3350, or SOFTLAX, MIRALAX, DULCOLAX BALANCE, CLEARLAX, OSMOLAX OR GLYCOLAX, GOLYTELY, GAVILYTE C, NULYTELY, GLYCOLAX, FORTRANS, TRILYTE, COLYTE, HALFLYTELY, SOFTLAX, LAX-A-DAY, CLEARLAX AND MOVIPREP). In some embodiments, the additional therapeutic agent is a laxative, including but not limited to stimulant laxatives (such as, for example, SENOKOT). Also provided are contact laxatives (e.g. oxyphenisatine, bisacodyl, dantron, phenolphthalein, castor oil, senna glycosides, cascara, sodium picosulfate, and bisoxatin) and bulk-forming laxatives (e.g. ispaghula, ethulose, sterculia, linseed, methylcellulose, triticum, and polycarbophil calcium). In some embodiments, the additional therapeutic agent is an enema, such as, for example, sodium laurylsulfate, sodium phosphate, bisacodyl, dantron, glycerol, oil, and sorbitol. Peripheral opioid antagonists such as, for example, alvimopan and methylnaltrexone, as well as prostaglandins such as, for example, lubiprostone are also additional therapeutic agents in some embodiments. Also, linaclotide, prucalopride, and tegaserod may be additional therapeutics.

In some embodiments, the additional therapeutic agent is an agent used for long-term pain and cramping, including but not limited to anticholinergics (antispasmodics), such as, for example, dicyclomine (BENTYL) and or antidepressants, including, for example, desipramine (such as, for example, NORPRAMIN), imipramine (TOFRANIL) or nortriptyline (PAMELOR), which are optionally administered at low doses. In low doses, they can help with pain caused by IBS.

In some embodiments, the additional therapeutic agent is fiber supplement, such as, for example, psyllium (METAMUCIL) or methylcellulose (CITRUCEL).

In some embodiments, the additional therapeutic agent is an agent useful for treating obesity. Illustrative agents include, but are not limited to, orlistat, loracaserin, phentermine-topiramate, sibutramine, rimonabant, exenatide, pramlintide, phentermine, benzphetamine, diethylpropion, phendimetrazine, bupropion, and metformin. In various embodiments, the additional agent is an agent that that interfere with the body's ability to absorb specific nutrients in food, such as orlistat, glucomannan, and guar gum. Agents that suppress appetite are also among the additional agents, e.g. catecholamines and their derivatives (such as phentermine and other amphetamine-based drugs), various anti-depressants and mood stabilizers (e.g. bupropion and topiramate), anorectics (e.g. dexedrine, digoxin). Agents that increase the body's metabolism are also among the additional agents. In some embodiments, additional agents may be selected from among appetite suppressants, neurotransmitter reuptake inhibitors, dopaminergic agonists, serotonergic agonists, modulators of GABAergic signaling, anticonvulsants, antidepressants, monoamine oxidase inhibitors, substance P (NK1) receptor antagonists, melanocortin receptor agonists and antagonists, lipase inhibitors, inhibitors of fat absorption, regulators of energy intake or metabolism, cannabinoid receptor modulators, agents for treating addiction, agents for treating metabolic syndrome, peroxisome proliferator-activated receptor (PPAR) modulators; and dipeptidyl peptidase 4 (DPP-4) antagonists. In some embodiments, additional agents may be selected from among amphetamines, benzodiazepines, sulfonyl ureas, meglitinides, thiazolidinediones, biguanides, beta-blockers, ACE inhibitors, diuretics, nitrates, calcium channel blockers, phenlermine, sibutramine, lorcaserin, cetilistat, rimonabant, taranabant, topiramate, gabapentin, valproate, vigabatrin, bupropion, tiagabine, sertraline, fluoxetine, trazodone, zonisamide, methylphenidate, varenicline, naltrexone, diethylpropion, phendimetrazine, repaglinide, nateglinide, glimepiride, pioglitazone, rosiglilazone, and sitagliptin.

In an embodiment, the additional therapeutic agent is an agent for treating pre-diabetes, diabetes, type II diabetes, insulin resistance, glucose intolerance, or hyperglycemia. Examples of drugs include, but are not limited to, alpha-glucosidase inhibitors, amylin analogs, dipeptidyl peptidase-4 inhibitors, GLP1 agonists, meglitinides, sulfonylureas, biguanides, thiazolidinediones (TZD), and insulin. Additional examples of such agents include bromocriptine and Welchol. Examples of alpha-glucosidase inhibitors include but are not limited to acarbose and miglitol. An example of an amylin analog is pramlintide. Examples of dipeptidyl peptidase-4 inhibitors include but are not limited to saxagliptin, sitagliptin, vildagliptin, linagliptin, and alogliptin. Examples of GLP1 agonist include but are not limited to liraglutide, exenatide, exenatide extended release. Examples of meglitinides include but are not limited to nateglinide, and repaglinide. Examples of sulfonylureas include but are not limited to chlorpropamide, glimepiride, glipizide, glyburide, tolazamide, and tolbutamide. Examples of biguanides include but are not limited to metformin, Riomet, Glucophage, Glucophage XR, Glumetza. Examples of thiazolidinedione include but are not limited to rosiglitazone and pioglitazone. Examples of insulin include but are not limited to Aspart, Detemir, Glargine, Glulisine, and Lispro. Examples of combination drugs include but are not limited to glipizide/metformin, glyburide/metformin, pioglitazone/glimepiride, pioglitazone/metformin, repaglinide/metformin, rosiglitazone/glimepiride, rosiglitazone/metformin, saxagliptin/metformin, sitagliptin/simvastatin, sitagliptin/metformin, linagliptin/metformin, alogliptin/metformin, and alogliptin/pioglitazone.

In another embodiment, the additional therapeutic agent is a probiotic. In some embodiments, enteric dietary formulations containing low residual material, such as pre-digested or basic amino acid formulations and other methods and products as described in U.S. Pat. No. 8,110,177 (the contents of which are incorporated herein by reference) may be employed. In a further embodiment, such low residual enteric dietary formulations may be formulated in low carbohydrate and low fat forms either with or without immediate or sustained release statins or red yeast rice which may be particularly useful for weight loss and diabetes. In various embodiments, the probiotic may comprise the following illustrative cells: E. coli Nissle 1917, a lactobacillus (e.g. acidophilus, Lactobacillus brevis, L. bulgaricus, L. plantarum, L. rhamnosus, Rhamnosus L. fermentum, L. caucasicus, L. helveticus, L. lactis, L. reuteri and L. casei) or a bifidobacteria (Bifidobacterium bifidum, B. infantis) Streptococcus thermophilus, and Enterococcus faecium. Other suitable probiotics and prebiotics are disclosed for example in R. Spiller, Aliment Pharmacol Ther 28, 385-396, the contents of which are hereby incorporated by reference in their entirety.

In some embodiments, a probiotic agent that optionally inhibits the growth of methanogens, for example, Bifidobacterium spp. or Lactobacillus species or strains, e.g., L. acidophilus, L. rhamnosus, L. plantarum, L. reuteri, L. paracasei subsp. paracasei, or L. casei Shirota, or probiotic Saccharomyces species, e.g., S. cerevisiae, is selected and/or administered. The probiotic agent that inhibits methanogenesis may be administered in a pharmaceutically acceptable ingestible formulation, such as in a capsule, or for some subjects, consuming a food supplemented with the inoculum is effective, for example a milk, yogurt, cheese, meat or other fermentable food preparation. Probiotic agents can inhibit the growth of methanogens, for example, by competing against methanogens for growth and thus reduce or inhibit the growth of methanogens.

### Methods of Treatment

In one aspect, the present invention provides methods of treating or preventing a methanogen-associated disorder by administering a statin, a statin analog or derivative, or a compound or formulation as described herein to the intestine (i.e., small and/or large intestine) in a subject in need thereof.

In some embodiments, the methanogen-associated disorder is a disease or disorder or condition caused by, resulted from, or related to one or more of the abnormal presence or absence of methanogens, abnormal levels of methanogens, overgrowth of methanogens, elevated levels of methanogenesis, elevated enteric methane levels, excessive hydrogen scavenging by hydrogen-consuming methanogens or colonization of methanogens in an abnormal location (e.g., in the small bowel rather than large bowel), either alone or in combination with non-methanogen syntrophic organisms.

Illustrative methanogen-associated disorders include, but are not limited to, enteric methanogen colonization, IBS, IBS-C, IBS-M, constipation, diabetes, type 2 diabetes, metabolic syndrome, insulin resistance, metabolic syndrome, obesity, constipation, chronic constipation, chronic intestinal pseudo-obstruction, systemic sclerosis, systemic lupus, erythematosus, dermatomysitis/polymyositis, polyarteritis nodosa, mixed connective tissue disorder, rheumatoid arthritis, spinal cord injury, Parkinson's disease, hypothyroidism/hypoparathyroidism, Hirschsprung's disease, Chagas' disease, intestinal hypoganglionosis, and Ehlers-Danlos Syndrome.

In one aspect, the present invention provides methods of reducing or eliminating the production and/or accumulation of methane in the GI tract by administering a statin, a statin analog or derivative, or a compound or formulation as described herein to the intestine (e.g. the small and/or large intestine) of a subject in need thereof. In another aspect, the present invention provides methods of reducing or eliminating methane, for example as produced by a methanogen in the GI tract by administering a statin, a statin analog or derivative, or a compound or formulation as described herein to the intestine (i.e., small and/or large intestine) of a subject in need thereof.

In various embodiments, the methanogen is a microorganism that produces methane as a metabolic byproduct. Methanogens are classified as archaea. Examples of methanogens include but are not limited to Methanobacterium bryantii, Methanobacterium formicum, Methanobrevibacter arboriphilicus, Methanobrevibacter gottschalkii, Methanobrevibacter ruminantium, Methanobrevibacter smithii, Methanocalculus chunghsingensis, Methanococcoides burtonii, Methanococcus aeolicus, Methanococcus deltae, Methanococcus jannaschii, Methanococcus maripaludis, Methanococcus vannielii, Methanocorpusculum labreanum, Methanoculleus bourgensis (Methanogenium olentangyi, Methanogenium bourgense), Methanoculleus marisnigri, Methanofollis liminatans, Methanogenium cariaci, Methanogenium frigidum, Methanogenium organophilum, Methanogenium wolfei, Methanomicrobium mobile, Methanopyrus kandleri, Methanoregula boonei, Methanosaeta concilii, Methanosaeta thermophila, Methanosarcina acetivorans, Methanosarcina barkeri, Methanosarcina mazei, Methanosphaera stadtmanae, Methanospirillium hungatei, Methanothermobacter defluvii (Methanobacterium defluvii), Methanothermobacter thermautotrophicus (Methanobacterium thermoautotrophicum), Methanothermobacter thermoflexus (Methanobacterium thermoflexum), Methanothermobacter wolfei (Methanobacterium wolfei), and Methanothrix sochngenii.

In one aspect, the present invention provides methods of reducing or eliminating the methane derived from Methanobrevibacter smithii in the GI tract. In another aspect, the present invention provides methods of reducing or eliminating methane, for example as produced by Methanobrevibacter smithii, in the GI tract by administering a statin, a statin analog or derivative, or a compound or formulation as described herein to the intestine (i.e., small and/or large intestine) in a subject in need thereof. In various embodiments, the present invention relates to the substantial reduction of methane gas in a subjects GI tract (e.g. eradication of intestinal methane). In some embodiments the present formulations and methods prevent the increase in levels of methane gas in a subject's GI tract. In some embodiments, the patient's GI methane levels (as assessed by methods described herein and methods known in the art) are reduced to about 1 ppm, or about 2 ppm, or about 3 ppm, or about 4 ppm, or about 5 ppm, or about 10 ppm, or about 15 ppm, or about 20 ppm, or about 25 ppm, or about 30 ppm, or about 35 ppm, or about 40 ppm, or about 45 ppm, or about 50 ppm, or about 55 ppm, or about 60 ppm, or about 65 ppm, or about 70 ppm, or about 75 ppm, or about 80 ppm, or about 85 ppm, or about 90 ppm, or about 100 ppm. In various embodiments, the present formulations and methods reduce the patient's GI methane levels to less than about 250 ppm, or less than about 225 ppm, or less than about 200 ppm, or less than about 175 ppm, or less than about 150 ppm, or less than about 125 ppm, or less than about 100 ppm, or less than about 50 ppm. In various embodiments, substantial reduction of methane gas is not accompanied by a substantial reduction in hydrogen gas.

In various embodiments, the present invention relates to the treatment of IBS, including IBS-C as described by ICD-10 (International Statistical Classification of Diseases and Related Health Problems, WHO edition). In various embodiments, the present invention relates to the treatment of irritable colon, as classified in ICD-10 as [K58]. IBS may include irritable bowel syndrome without diarrhea, as classified in ICD-10 as [K58.9]. Irritable bowel syndrome without diarrhea may also include irritable bowel syndrome not otherwise specified (NOS). Further, the diseases as classified in ICD-10 as K59 are also included (e.g. constipation; K59.1 Functional diarrhea; K59.2 Neurogenic bowel, not elsewhere classified; K59.3 Megacolon, not elsewhere classified (including dilatation of colon, toxic megacolon, megacolon in Chagas disease (B57.3), congenital (aganglionic) (Q43.1), and Hirschsprung disease (Q43.1)); K59.4 Anal spasm (including Proctalgia fugax); K59.8 Other specified functional intestinal disorders (including any of colon) and K59.9 Functional intestinal disorder, unspecified).

In various embodiments, the present invention relates to the treatment of spastic colon, nervous colitis, mucous colitis, functional colitis or colonic neurosis. In various embodiments, the present invention relates to the treatment of diseases that have been described as sigma elongatum mobile, cecum mobile, chronic colitis, splanchnoptosia and the like. Typological classification of the disease generally include convulsive large bowel, diarrhea nervosa and colica mucosa, and the disease may also be classified in convulsive constipation type, atonic constipation type, intestinal gas syndrome, or chronic celiopathy.

Furthermore, IBS may also include cholangiodyskinesia, gastric emptying hypofunction, hysteric globus, non-specific esophagus functional abnormalities, nervous vomiting, recurrent abdominal pain, simple constipation, chronic idiopathic constipation and the like. As diagnostic criteria of IBS those of NIH, Manning, Cook et al. and the like are suitable (see Asakura, Clinical Digestive Internal Medicine. 8 (8): 1373-1381 (1993), the contents of which are hereby incorporated by reference in their entirety).

In various embodiments, the present invention relates to the treatment of IBS, including IBS-C of varying stages or severity. In one embodiment, stages or severity of the IBS may be evaluated with a health-related quality of life (HRQoL) evaluation. In some embodiments, the stage or severity of the disease in the patient to be treated is assessed by an evaluation of one or more of patient pain, distension, bowel dysfunction and quality of life/global well-being.

In some embodiments, the stage or severity of the disease in the patient to be treated is assessed by the Rome Scale (for the last 3 months with symptom onset at least 6 months prior to diagnosis: recurrent abdominal pain or discomfort (e.g. uncomfortable sensation not described as pain.) at least 3 days/month in the last 3 months associated with two or more of improvement with defecation, onset associated with a change in frequency of stool, and onset associated with a change in the form (appearance) of stool.

In some embodiments, the stage or severity of the disease in the patient to be treated is assessed by the Kruis scale (Gastroenterology 87: 1-7, the contents of which are hereby incorporated by reference). This scale incorporates both the “cardinal” symptoms (pain, bloating, altered bowel function) and “red flag” signs of potential underlying organic disease that would thus exclude an IBS diagnosis. IBS is diagnosed if the sum of scores >44. See, e.g., Table 1.

TABLE 1
Kruis Scoring System, IBS is diagnosed if the sum of scores >44

| Parameter | Score |
|---|---|
| Signs | |
| Pain, flatulence, or bowel irregularity | 34 |
| Duration of symptoms > 2 yr | 16 |
| Description of abdominal pain (Scale from burning to “not so bad”) | 23 |
| Alternating diarrhea and constipation | 14 |
| Red Flags | |
| Abnormal physical findings or history pathognomonic of other | −47 |
| disease | |
| ESR > 10 mm/h | −13 |
| WBC > ×10⁹ | −50 |
| Anemia | −98 |
| History of blood in stool | −98 |

In some embodiments, the patient is evaluated with the assessment described in Francis, et al Aliment Pharmacol Ther 1997; 11: 395-402, the contents of which are hereby incorporated by reference in their entirety. For instance, a scoring system based on patient ranking of pain, distension, bowel dysfunction and quality of life/global well-being on a scale of up to 500 is used. Mild, moderate and severe cases were indicated by scores of 75 to 175, 175 to 300 and >300. In some embodiments, the patient of the present invention has a score of 75 to 175. In some embodiments, the patient of the present invention has a score of 175 to 300. In some embodiments, the patient of the present invention has a score of >300. In some embodiments the scales are described in Wong and Drossman (Expert Rev. Gastroenterol. Hepatol. 4(3), (2010), the contents of which are hereby incorporated by reference in their entirety). For example, in some embodiments, the patients of the present invention are evaluated for the parameters of dysphoria, activity interference, body image, health worry, food avoidance, social reaction, and sexual relationships and optionally scored on a 0-100 as described on the Patrick scale; and/or the patients of the present invention are evaluated for the parameters of daily activities, emotional impact, family relations, food, sleep and fatigue, social impact, sexual relations symptoms and optionally scored on a 0-216 as described on the Groll scale; the patients of the present invention are evaluated for the parameters of activities, anxiety, diet, sleep, discomfort, health perception, disease coping and stress and optionally scored on a 0-100 as described on the Chassany scale; the patients of the present invention are evaluated for the parameters of emotional health, mental health, sleep, energy, physical functioning, diet, social role, physical role, and sexual relations and optionally scored on a 0-100 as described on the Hahn scale; and/or the patients of the present invention are evaluated for the parameters of bowel symptoms, fatigue, activity impairment, emotional dysfunction and optionally scored as domain average scores (calculated by dividing the domain sum score by the number of items: range 1-7) as described on the Wong scale.

In some embodiments, patients may be stratified based on one or more of methane detection (e.g. via breath test) and methanogen detection (e.g. via PCR, e.g. qPCR). In some embodiments, the patient is considered methane breath test positive if the subject presents with greater than about 3 ppm methane. In some embodiments, the patient of the present invention has greater than about 10⁴, or about 10⁵, or about 10⁶ copies of M. smithii per grams of wet stool. In some embodiments, the patient of the present invention is defined by a measurement of the fractional methanogen contribution to the total microbial content of the feces. In some embodiments, the patient has greater than about 0.5%, or about 0.6%, or about 0.7%, or about 0.8%, or about 0.9%, or about 1.0%, or about 1.1%, or about 1.2%, or about 1.3%, or about 1.4%, or about 1.5%, or about 2.5% M. smithii fraction of the total microbial content of the feces.

In some embodiments, methods of the present invention treat or prevent constipation. Constipation may be associated with, for example, chemotherapy, vinca alkaloids, oxaliplatins, taxanes, thalidomide, opioids, sedatives, anticholinergics, gastrointestinal antispasmodics, antiparkinsonism agents, antidepressants, phenothiazines, calcium- and aluminum-based antacids, diuretics, tranquilizers, sleeping medications, general anesthesia, pudendal blocks, inadequate fluid intake, excessive use of laxatives and/or enemas, prolonged immobility, inadequate exercise, spinal cord injury or compression, fractures, fatigue, weakness, inactivity, bed rest, cardiac problems, diverticulitis, neurological lesions, cerebral tumors, spinal cord injury, spinal cord compression, paraplegia, cerebrovascular accident with paresis, weak abdominal muscles, hypothyroidism, lead poisoning, uremia, dehydration, hypercalcemia, hypokalemia, hyponatremia, anorexia, immobility, antidepressants, inability to increase intra-abdominal pressure, emphysema, neuromuscular impairment of the diaphragm, neuromuscular impairment of abdominal muscles, abdominal hernias, malnutrition, cachexia, anemia, carcinoma, and senility.

In various embodiments, the constipation is associated with IBS. But, the present invention, in some embodiments, can also relate to chronic functional constipation.

In various embodiments, the present invention relates to the treatment of increased visceral hypersensitivity. In various embodiments, the present invention relates to the treatment of one or more of stomachaches, pain, nausea, straining, and bloating and/or gas. The present formulations and methods also treat one or more of as hard stools, infrequent stools, difficulty or straining at stools, feeling of being unable to completely empty during a bowel movement, and the sensation of wanting to go but not being able to.

In various embodiments, the present invention relates to the treatment for diabetes (type 1 or type 2) and/or glucose intolerance. In some embodiments, the present invention relates to a method for treating patient at risk of diabetes, one or more of insulin resistance, prediabetes, impaired fasting glucose (IFG), impaired glucose tolerance (IGT), and acanthosis nigricans.

In some embodiments, methods for inducing weight loss or preventing weight gain (or treating or preventing obesity or inducing weight loss or preventing weight gain in a patient that does not substantially change caloric intake), comprising administering a statin, statin analog or derivative, or a compound or formulation of the present invention are provided. Patients may have undertaken or will undertake a surgery of the digestive system; be greater than about 80-100 pounds overweight; have a BMI of greater than about 35 kg/m²; or have a health problem related to obesity

In embodiments, administration of the statin, statin analog or derivative, or compound or formulation of the present invention does not confer cholesterol-lowering cardiovascular effects associated with systemic administration. For example, the present formulations and methods may avoid or reduce a subject's systemic exposure to the statin or statin analog or derivative. For example, the present formulations and methods may provide an average reduction of less than about 20%, about 19%, about 18%, about 17%, about 16%, about 15%, about 14%, about 13%, about 12%, about 11%, about 10%, about 9%, about 8%, about 7%, about 6%, about 5%, about 4%, about 3%, or about 2% in serum LDL-C levels after treatment.

In some embodiments, the patient is one who does not require statins for their cardiovascular therapeutic uses. In some embodiments, the patient is one who does not require statins for their cardiovascular therapeutic uses and is methane-positive (e.g. as assessed by the methods described herein such as the methane breath test and qPCR).

By maximizing retention of the statins to the intestines, the methods of the invention also minimize the side effects associated with systemic release. For example, the present method prevents and/or minimizes various adverse effects associated with statin usage including, muscle-associated adverse effects, such as myositis, myalgia, rhabdomyolysis, drug-drug-interactions, cognitive effects, increased cancer risk, increases in liver enzymes, hemorrhagic stroke, increase in blood glucose levels, sleep disorders, peripheral neuropathy, sexual dysfunction, thyroid dysfunction, renal toxicity, irritability, shortness of breath, hyperkalemia, weight gain, neurodegenerative disease, pancreatitis, liver pathology, mitochondrial syndromes, dermatologic conditions, dry mouth, cataracts, olfaction, hematological and bone marrow adverse effects, hypotension, gastrointestinal adverse effects, including, ulcerative colitis and gastric ulceration, fatigue and headache. In some embodiments, the methods of the invention also minimizes the following side effects associated with systemic release of statins: muscle pain, tenderness, or weakness, lack of energy, weakness, fever, dark colored urine, jaundice, pain in the stomach, including the upper right part of the stomach, nausea, unusual bleeding or bruising, loss of appetite, flu-like symptoms, rash, hives, itching, difficulty breathing or swallowing, and swelling of the face, throat, tongue, lips, eyes, hands, feet, ankles, or lower legs, hoarseness.

Accordingly, the statin, statin analog or derivative, or compound or formulation as described herein may be used to target subjects where systemic statin levels are undesirable. In one embodiment, the subject may be women and children who are otherwise healthy and have no need for a cardiovascular medicine (as characterized, for example, as having low or zero myocardial event risk factors as per the ATP III Guideline). In another embodiment, the subject may be a child with IBS-C who has no need for a cholesterol-lowering agent. In such embodiments, administration of the statin, statin analog or derivative, or compound or formulation of the present invention results in an average reduction of less than about 20%, about 19%, about 18%, about 17%, about 16%, about 15%, about 14%, about 13%, about 12%, about 11%, about 10%, about 9%, about 8%, about 7%, about 6%, about 5%, about 4%, about 3%, or about 2% in serum LDL-C levels after treatment.

The statin, statin analog or derivative, or compound or formulation of the present invention may also be utilized as part of a treatment regimen wherein a subject is provided with an initial anti-methanogenic therapy followed by a chronic anti-methanogenic or methane-reducing and/or eliminating maintenance therapy.

The initial anti-methanogenic therapy may employ agents other than statins such as, for example, antibiotics which eradicate the methanogens. For example nitroimidazoles such as metronidazole, metronidazole esters and/or isomers or hydrophobic imidazole derivatives or rifaximin or neomycin sufficient to eradicate, substantially reduce, or reduce the enteric methanogen colonization may be used. Such initial therapy may be for 1, 2, 3, 4, 5, 6, 7, 8, 9, 10, 14, 28, 42, 56, 60, 90, 120 or 180 days or more. Examples of antibiotics include but are not limited to aminoglycosides (e.g., amikacin, gentamicin, kanamycin, neomycin, netilmicin, streptomycin, tobramycin, paromomycin), ansamycins (e.g., geldanamycin, herbimycin), carbacephems (e.g., loracarbef), carbapenems (e.g., ertapenem, doripenem, imipenem, cilastatin, meropenem), cephalosporins (e.g., first generation: cefadroxil, cefazolin, cefalotin or cefalothin, cefalexin; second generation: cefaclor, cefamandole, cefoxitin, cefprozil, cefuroxime; third generation: cefixime, cefdinir, cefditoren, cefoperazone, cefotaxime, cefpodoxime, ceftazidime, ceftibuten, ceftizoxime, ceftriaxone; fourth generation: cefepime; fifth generation: ceftobiprole), glycopeptides (e.g., teicoplanin, vancomycin), macrolides (e.g., azithromycin, clarithromycin, dirithromycin, erythromycin, roxithromycin, troleandomycin, telithromycin, spectinomycin), monobactams (e.g., aztreonam), penicillins (e.g., amoxicillin, ampicillin, azlocillin, carbenicillin, cloxacillin, dicloxacillin, flucloxacillin, mezlocillin, meticillin, nafcillin, oxacillin, penicillin, piperacillin, ticarcillin), antibiotic polypeptides (e.g., bacitracin, colistin, polymyxin b), quinolones (e.g., ciprofloxacin, enoxacin, gatifloxacin, levofloxacin, lomefloxacin, moxifloxacin, norfloxacin, ofloxacin, trovafloxacin), rifamycins (e.g., rifampicin or rifampin, rifabutin, rifapentine, rifaximin), sulfonamides (e.g., mafenide, prontosil, sulfacetamide, sulfamethizole, sulfanilamide, sulfasalazine, sulfisoxazole, trimethoprim, trimethoprim-sulfamethoxazole (co-trimoxazole, “tmp-smx”), and tetracyclines (e.g., demeclocycline, doxycycline, minocycline, oxytetracycline, tetracycline) as well as arsphenamine, chloramphenicol, clindamycin, lincomycin, ethambutol, fosfomycin, fusidic acid, furazolidone, isoniazid, linezolid, metronidazole, mupirocin, nitrofurantoin, platensimycin, pyrazinamide, quinupristin/dalfopristin combination, and tinidazole.

Following the initial therapy, a subject may be placed on maintenance therapy in order to maintain reduced methanogen and/or methane levels. In some embodiments, the maintenance therapy utilizes a statin, a statin analog or derivative, or a compound or formulation of the present invention. In an embodiment, the initial therapy includes an antibiotic followed by a chronic maintenance regimen of low dose statin, statin analog or derivative, or compound or formulation of the invention. In various embodiments, the maintenance regiment may be administered for at least 1 week, at least 2 weeks, at least 3 weeks, at least 4 weeks, at least one month, at least two months, at least three months, at least four months, at least five months, at least six months, at least seven months, at least eight months, at least nine months, at least ten months, at least eleven months, at least 1 year, at least 2 years, at least 3 years, at least 4 years, at least 5 years, at least 10 years, or indefinitely.

In some embodiments, the statin, statin analog or derivative, or compound or formulation of the present invention may be utilized solely for chronic maintenance therapy. In various embodiments, the present invention provides a method of treating previously methane positive patients who do not have one or more of cardiovascular disease, an LDL level of 190 mg/dL or higher, Type 2 diabetes who are between 40 and 75 years of age, an estimated 10-year risk of cardiovascular disease of 7.5 percent or higher who are between 40 and 75 years of age with a statin, a statin analog or derivative, or a compound or formulation as described herein in order to maintain their methane negative status. Accordingly, in some embodiments, the statin, statin analog or derivative, or compound or formulation as described herein finds use as a prevention measure in a high risk patient.

In various embodiments, the subject is a mammal, e.g., a human, mouse, rat, guinea pig, dog, cat, horse, cow, pig, rabbit, sheep, or non-human primate, such as a monkey, chimpanzee, or baboon. In some embodiments, the subject is a non-human animal, and therefore the invention pertains to veterinary use. In a specific embodiment, the non-human animal is a livestock animal as described herein.

In various embodiments, methods of the invention are useful in treatment a human subject. In some embodiments, the human is a pediatric human. In other embodiments, the human is an adult human. In other embodiments, the human is a geriatric human. In other embodiments, the human may be referred to as a patient. In some embodiments, the human is a female. In some embodiments, the human is a male.

In embodiments, the human has an age in a range of from about 1 to about 18 months old, from about 18 to about 36 months old, from about 1 to about 5 years old, from about 5 to about 10 years old, from about 10 to about 15 years old, from about 15 to about 20 years old, from about 20 to about 25 years old, from about 25 to about 30 years old, from about 30 to about 35 years old, from about 35 to about 40 years old, from about 40 to about 45 years old, from about 45 to about 50 years old, from about 50 to about 55 years old, from about 55 to about 60 years old, from about 60 to about 65 years old, from about 65 to about 70 years old, from about 70 to about 75 years old, from about 75 to about 80 years old, from about 80 to about 85 years old, from about 85 to about 90 years old, from about 90 to about 95 years old or from about 95 to about 100 years old. In an embodiment, the human is a child. In one embodiment, the human is female.

### Patient Selection and Evaluation/Methods to Determine Methanogen Levels

In various embodiments, the present methods provide for a manner for selecting patients who are likely to respond to treatment with the statin, statin analog or derivative, or compound or formulation of the invention. In some embodiments, the present methods provide for a manner for selecting patients who are likely to respond to treatment with a lactone form of a statin or statin analog or derivative or any of the compounds disclosed herein. Such patients include those suffering from a methanogen-associated disorder as described herein. In an embodiment, the patient is a patient with a GI disorder such as an irritable bowel syndrome patient.

In embodiments, patients who are likely to respond to treatment with the statin, statin analog or derivative, or compound or formulation of the invention have GI microbes which express a greater level of methanogenesis-related F₄₂₀-dependent enzymes than those who do not respond to treatment, e.g. patients having biological samples with greater levels of methanogenesis-related F₄₂₀-dependent enzymes than those who do not respond to treatment. In some embodiments, patients who are likely to respond to treatment with the statin, statin analog or derivative, or compound or formulation of the invention have GI microbes which express a high level of methanogenesis-related F₄₂₀-dependent enzymes. In an embodiment, patients who are likely to respond to treatment with the statin, statin analog or derivative, or compound or formulation of the invention have GI microbes which express a high level of mtd/A5UMI1 (F₄₂₀-dependent methylenetetrahydromethanopterin dehydrogenase). Accordingly, in embodiments, patient who are likely to benefit from treatment using the statin, statin analog or derivative, or compound or formulation of the invention have GI microbes which express a high level of methanogenesis-related F₄₂₀-dependent enzymes (e.g., high level of mtd/A5UMI1). In some embodiments, methods of the invention are practiced on patients who have GI microbes which express a high level of methanogenesis-related F₄₂₀-dependent enzymes (e.g., high level of mtd/A5UMI1).

In such embodiments, biological samples are obtained from the patient prior to treatment for quantitative analysis. Exemplary biological samples include stool, mucosal biopsy from a site in the gastrointestinal tract, aspirated liquid from a site in the gastrointestinal tract, sputum, blood, or combinations thereof. Quantitative analysis may include, for example, quantitative polymerase chain reaction (qPCR) or other molecular biology approaches described herein. In various quantitative analyses are undertaken to determine the level of F₄₂₀-dependent enzyme.

Methods of “quantitative” amplification are well known to those of skill in the art. For example, quantitative PCR involves simultaneously co-amplifying a known quantity of a control sequence using the same primers. This provides an internal standard that may be used to calibrate the PCR reaction. Detailed protocols for quantitative PCR are provided in, for example, Innis, et al. (1990) PCR Protocols, A Guide to Methods and Applications, Academic Press, Inc. N.Y.). Measurement of DNA copy number at microsatellite loci using quantitative PCR analysis is described in, for example, Ginzonger, et al. (2000) Cancer Research 60:5405-5409. The known nucleic acid sequence for the genes is sufficient to enable one of skill in the art to routinely select primers to amplify any portion of the gene. Fluorogenic quantitative PCR may also be used in the methods of the invention. In fluorogenic quantitative PCR, quantitation is based on amount of fluorescence signals, e.g., TaqMan and Sybr green.

Other suitable amplification methods include, but are not limited to, ligase chain reaction (LCR) (see, for example, Wu and Wallace (1989) Genomics 4: 560, Landegren, et al. (1988) Science 241:1077, and Barringer et al. (1990) Gene 89: 117), transcription amplification (Kwoh, et al. (1989) Proc. Natl. Acad. Sci. USA 86: 1173), self-sustained sequence replication (Guatelli, et al. (1990) Proc. Nat. Acad. Sci. USA 87: 1874), dot PCR, and linker adaptcr PCR, etc.

In still other embodiments of the methods provided herein, sequencing of individual nucleic acid molecules (or their amplification products) is performed. In one embodiment, a high throughput parallel sequencing technique that isolates single nucleic acid molecules of a population of nucleic acid molecules prior to sequencing may be used. Such strategies may use so-called “next generation sequencing systems” including, without limitation, sequencing machines and/or strategies well known in the art, such as those developed by Illumina/Solexa (the Genome Analyzer; Bennett et al. (2005) Pharmacogenomics, 6:373-20 382), by Applied Biosystems, Inc. (the SOLiD Sequencer; solid.appliedbiosystems.com), by Roche (e.g., the 454 GS FLX sequencer; Margulies et al. (2005) Nature, 437:376-380; U.S. Pat. Nos. 6,274,320; 6,258,568; 6,210,891) and others. Other sequencing strategies such as stochastic sequencing (e.g., as developed by Oxford Nanopore) may also be used, e.g., as described in International Patent Publication No. WO/2010/004273. In still other embodiments of the methods provided herein, deep sequencing can be used to identify and quantify the methanogenesis-related enzymes, the methanogen, or methanogen syntrophic microorganism. These techniques are known in the art.

In embodiments, methods are provided that allow for patient evaluation, e.g., diagnosis or prognosis, based on a patient's status of F₄₂₀-dependent enzyme status (e.g., methanogenesis-related enzymes). Accordingly, the various methods of treatment described herein may involve a step of profiling a patient for F₄₂₀-dependent enzyme status (e.g., methanogenesis-related enzymes). Profiling may be conducted by obtaining a biological sample from the patient and performing a quantitative analysis such as qPCR as described herein. In various embodiments, treatments using the statin, statin analog or derivative, or compound or formulation of the invention result in a reduction in the levels of methanogenesis-related enzyme such as F₄₂₀-dependent enzyme. In an embodiment, treatments using the statin, statin analog or derivative, or compound or formulation of the invention result in a reduction of the levels of mtd/A5UMI1.

In some embodiments, a patient's methane level may be evaluated. Intestinal methanogen and/or methane levels can be determined by breath tests that measure breath methane levels. Breath testing may be utilized to identify subjects who are “methane-positive” and who can potentially benefit from methods of the present invention. Further, breath testing can also be used to monitor the efficacy of treatment. Breath testing analysis methods and equipment are known in the art (see, for example, International Patent Publication WO/2014/152754, the entire contents of which are incorporated by reference herein). Examples of such equipment include, for example, the QuinTron BreathTracker gas chromatographic (GC) analyzer or the QuinTron BreathTracker device (QuinTron Instrument Company, Inc., Milwaukee, Wis.).

Further, abnormal lactulose breath test results are common in subjects with IBS and therefore the present invention provides for the use of lactulose breath tests in evaluating patients. In some embodiments, a patient is evaluated with a lactulose breath test before and/or after administration with the statin, statin analog or derivative, or compound or formulations described herein.

In general, individuals having a breath methane level of at least about 3 ppm are generally associated with methanogen-associated disorders and are likely to benefit from methods of the present invention. Alternatively, methods of the invention may be practiced on subjects having a breath methane level of at least 1 ppm, at least 1.5 ppm, at least 2 ppm, at least 2.5 ppm, at least 3 ppm, at least 3.5 ppm, at least 4 ppm, at least 5 ppm, at least 6 ppm, at least 7 ppm, at least 8 ppm, at least 9 ppm, at least 10 ppm.

One method for measuring methanogen levels involves calculation of a subject's breath methane area under the curve (BM-AUC). This method involves obtaining multiple breath samples averaging about 15 minutes apart for a period of about 90 minutes, or about 120 minutes, or for up to 4 hours or more at potentially less frequent intervals. The time period results are used to calculate a person's BM-AUC. For example, a subject may undergo a such as lactulose, xylose, lactose, or glucose breath test after a 12 hour fast. The breadth test may comprise a baseline breath measurement after which the subject ingests about 10 g of such as lactulose, xylose, lactose, or glucose. Following lactulose ingestion, the subject is then asked to provide a breath sample about every 15 minutes for about 90 to about 120 minutes to determine methane production. BM-AUC may be utilized for more precisely determining and monitoring, for example, the efficacy of the anti-methanogenic therapy. BM-AUC measurements could also be utilized to segregate “methane positive” from “methane negative” subjects for improved clinical decision making. BM-AUC may be compared to or utilized with measurement of methanogen levels in stool samples via PCR, e.g. qPCR. Alternatively, measurement of methanogen levels in stool samples via PCR, e.g. qPCR may supplant the use of a breath test. More precise techniques may also involve measurement of breath methane taking into account and subtracting ambient methane levels.

Spot breath methane analysis via commercially available equipment such as BreathTracker may be used in discriminating “methane-positive” from “methane-negative” individuals, and monitoring the success, failure, dose titration, dosing schedule (daily or non-daily, for example) of the statin, statin analog or derivative, or compound or formulation of the invention. For example, the lowest minimum effective dose may be identified as such. Additional instruments and techniques for measuring methane levels may include cavity enhanced absorption techniques such as a LGR-FMR methane measurement instrument having a range as low as 0.01 ppm (Los Gatos Research, Inc., Mountain View, Calif.), wavelength-scanned cavity down-ring spectroscopy, carbon isotope analysis (G2132-i13C, Picarro, Inc, Santa Clara, Calif.), gas chromatography, mass spectroscopy, membrane extracted carbon isotope analysis (Pollock, 2012 GSA Annual Meeting, “Membrane Extracted Carbon Isotope Analysis Of Dissolved Methane”), headspace gas chromatography with FID detector and GC combustion with IRMS instruments, for example. Other instruments having the ability to measure low concentration breath methane levels at higher precision than the clinical validated instrument marketed as the QuinTron BreathTracker include high precision breath methane analysis (HPBMA). Use of HPBMA may be used to test spot breath methane levels or in BM-AUC form.

In some embodiments, detection of hydrogen quantity and methane quantity is by gas chromatography with mass spectrometry and/or radiation detection to measure breath emissions of isotope-labeled carbon dioxide, methane, or hydrogen, after administering an isotope-labeled substrate that is metabolizable by gastrointestinal bacteria but poorly digestible by the human host, such as lactulose, xylose, mannitol, or urea (e.g., G. R. Swart and J. W. van den Berg, ¹³C breath test in gastrointestinal practice, Scand. J. Gastroenterol. [Suppl.] 225:13-18 [1998]; S. F. Dellert et al., The ¹³C-xylose breath test for the diagnosis of small bowel bacterial overgrowth in children, J. Pediatr. Gastroenterol. Nutr. 25(2): 153-58 [1997]; C. E. King and P. P. Toskes, Breath tests in the diagnosis of small intestinal bacterial overgrowth, Crit. Rev. Lab. Sci. 21(3):269-81 [1984]). A poorly digestible substrate is one for which there is a relative or absolute lack of capacity in a human for absorption thereof or for enzymatic degradation or catabolism thereof.

Suitable isotopic labels include ¹³C or ¹⁴C. For measuring methane suitable isotopic labels can also include ²H and ³H or ¹⁷O and ¹⁸O, as long as the substrate is synthesized with the isotopic label placed in a metabolically suitable location in the structure of the substrate, i.e., a location where enzymatic biodegradation by intestinal microflora results in the isotopic label being sequestered in the gaseous product. If the isotopic label selected is a radioisotope, such as ¹⁴C, ³H, or ¹⁵O, breath samples can be analyzed by gas chromatography with suitable radiation detection means (e.g., Chang et al., Increased accuracy of the carbon-14 D-xylose breath test in detecting small-intestinal bacterial overgrowth by correction with the gastric emptying rate, Eur. J. Nucl. Med. 22(10): 1118-22 [1995]; King and Toskes, Comparison of the 1-gram [¹⁴C]xylose, 10-gram lactulose-H₂, and 80-gram glucose-H₂ breath tests in patients with small intestine bacterial overgrowth, Gastroent. 91(6):1447-51 [1986]; A. Schneider et al., Value of the ¹⁴C-D-xylose breath test in patients with intestinal bacterial overgrowth, Digestion 32(2):86-91 [1985]).

In embodiments, treatments using the statin, statin analog or derivative, or compound or formulation of the invention result in a reduction of breath methane level of at least about 1 ppm, at least about 2 ppm, at least about 3 ppm, at least about 4 ppm, at least about 5 ppm, at least about 6 ppm, at least about 7 ppm, at least about 8 ppm, at least about 9 ppm, at least about 10 ppm, at least about 20 ppm, at least about 30 ppm, at least about 40 ppm, at least about 50 ppm, at least about 60 ppm, at least about 70 ppm, at least about 80 ppm, at least about 90 ppm, at least about 100 ppm, at least about 110 ppm, at least about 120 ppm, at least about 130 ppm, at least about 140 ppm, at least about 150 ppm, at least about 160 ppm, at least about 170 ppm, at least about 180 ppm, at least about 190 ppm, at least about 200 ppm, at least about 210 ppm, at least about 220 ppm, at least about 230 ppm, at least about 240 ppm, and at least about 250 ppm.

The samples used for the present invention include a patient's breath. In various embodiments, measurement of methanogen levels in stool samples via PCR, e.g. qPCR or other molecular biology approaches as described herein. Further, aspirates of the fluid in the GI tract may be analyzed for methanogen and/or methane levels. Also mucosal biopsies from a site in the gastrointestinal tract may be analyzed for methanogen and/or methane levels.

### Livestock Applications

In various embodiments, the present invention provides methods of modifying a methanogenesis-related F₄₂₀-dependent enzyme in a livestock animal such as a ruminant. The term “ruminant” as used herein refers to any artiodactyl mammal of the suborder Ruminantia. Exemplary ruminants include cattle, calf, cow, goat, sheep, giraffe, bison, yak, water buffalo, deer, camel, alpaca, llama, wildebeest, antelope, pronghorn or nilgai. In an embodiment, the ruminant is cattle. In another embodiment, the ruminant is a goat or a sheep.

In various embodiments, the present invention provides methods for reducing methane production in ruminants comprising administering to a ruminant the statin, statin analog or derivative, or compound or formulation of the invention. The ruminant digestive tract is made up of four gastric compartments, the rumen, the reticulum, the abomasum and the omasum. The largest of these is the rumen. The rumen functions as a fermentation compartment. It contains large populations of microorganisms including methane producing archaea such as methanogens, which break down the plant material. Methane is expelled out from rumen through cructation. In various embodiments, methods of the invention reduces methane production from ruminants by at least about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, or about 95%.

In various embodiments, the present invention provides methods of reducing or eliminating the methane derived from Methanobrevibacter ruminantium. In another aspect, the present invention provides methods of reducing or eliminating methane, for example as produced by Methanobrevibacter ruminantium by administering a statin, a statin analog or derivative, or a compound or formulation as described herein to a ruminant in need thereof.

In various embodiments, the ruminants are fed with a feed composition comprising the statin, statin analog or derivative, or compound or formulation of the invention. In some embodiments, the statin, statin analog or derivative, or compound or formulation of the invention (for example, in the form of a capsule or tablet) may be added and mixed in with a standard feed compositions. The shape of the feed composition according to the present invention may be in any form of a conventional feed composition, such as a powder and a pellet. The feed composition according to the present invention can additionally contain other feed ingredients supplements and additives such as vitamins, enzymes, mineral salts, ground cereals, protein-containing components, carbohydrate-containing components, wheat middlings and/or brans.

It will be appreciated that the actual amount of the statin, statin analog or derivative, or compound disclosed herein to be administered to the ruminants will vary according to the particular compound, the particular dosage form, and the mode of administration. Many factors that may modify the action of the statin, statin analog or derivative, and compound disclosed herein (e.g., body weight, gender, diet, time of administration, route of administration, rate of excretion, condition of the ruminant, drug combinations, genetic disposition and reaction sensitivities) can be taken into account by those skilled in the art.

### Kits

The present invention is also directed to a kit for modulating F₄₂₀-dependent enzymes (e.g., specific methanogenesis pathway enzymes). In various embodiments, the kits may be utilized for the treatment of a methanogen-associated disorder. The kit is an assemblage of materials or components, including at least one statin, statin analog or derivative, and compound disclosed herein or formulation described herein. The kit may further include materials and components for the quantification of methanogens. The exact nature of the components configured in the kit depends on its intended purpose. In one embodiment, the kit is configured for the purpose of treating human subjects. In another embodiment, the kit is configured for treating ruminants.

Instructions for use may be included in the kit. Instructions for use typically include a tangible expression describing the technique to be employed in using the components of the kit to affect a desired outcome, such as to treat a disorder associated with methanogens. Optionally, the kit also contains other useful components, such as, diluents, buffers, pharmaceutically acceptable carriers, syringes, catheters, applicators, pipetting or measuring tools, bandaging materials or other useful paraphernalia as will be readily recognized by those of skill in the art.

The materials and components assembled in the kit can be provided to the practitioner store in any convenience and suitable ways that preserve their operability and utility. For example, the components can be provided at room, refrigerated or frozen temperatures. The components are typically contained in suitable packaging materials. In various embodiments, the packaging material is constructed by well-known methods, preferably to provide a sterile, contaminant-free environment. The packaging material may have an external label which indicates the contents and/or purpose of the kit and/or its components.

### Definitions

The term “alkyl” as used herein, refers to saturated, straight- or branched-chain hydrocarbon radicals. In some embodiments, the alkyl group may consist of 1 to 12 carbon atoms, e.g. 1 carbon atom, 2 carbon atoms, 3 carbon atoms, 4 carbon atoms etc., up to and including 12 carbon atoms. Illustrative alkyl groups include, but are in no way limited to, methyl, ethyl, propyl, isopropyl, n-butyl, iso-butyl, sec-butyl isobutyl, tertiary butyl, pentyl, isopentyl, neopentyl, hexyl, septyl, octyl, nonyl and decyl. Examples of such alkyl radicals include but are not limited to methyl, ethyl, propyl, isopropyl, n-butyl, tert-butyl, neopentyl, n-hexyl, n-propyl, n-octyl, n-decyl and n-dodecyl radicals.

The term “alkenyl” as used herein, refers to branched, unbranched or cyclic hydrocarbons, or combination thereof, having one or more carbon-carbon double bond. In some embodiments, the alkenyl group may contain from 2 carbon atoms to 12 carbon atoms, e.g., the alkenyl group may consist of 2 carbon atoms, 3 carbon atoms, 4 carbon atoms etc., up to and including 12 carbon atoms.

The term “cycloalkyl” as used herein, refers to a monocyclic or polycyclic radical that contains carbon and hydrogen, and may be saturated, or partially unsaturated. In some preferred embodiments, cycloalkyl groups include groups having from 3 to 12 ring atoms (i.e. (C₃₋₁₂)cycloalkyl or C(₃₋₁₂)cycloalkyl). Illustrative examples of cycloalkyl groups include, but are not limited to cyclopropyl, cyclobutyl, cyclopentyl, cyclopentenyl, cyclohexyl, cyclohexenyl, cycloseptyl, cyclooctyl, cyclononyl, cyclodecyl, norbornyl, and the like. Unless stated otherwise specifically in the specification, a cycloalkyl group is optionally substituted by one or more described as suitable substituents for alkyl and cycloalkyl respectively.

The terms “heteroalkyl”, “heteroalkenyl” and “heteroalkynyl” include optionally substituted alkyl, alkenyl and alkynyl radicals and which have one or more skeletal chain atoms selected from an atom other than carbon, e.g., oxygen, nitrogen, sulfur, phosphorus or combinations thereof. A heteroalkyl group may be substituted with one or more substituents which are described herein as suitable substitution groups.

“Heterocyclic” refers to a stable 3- to 18-membered non-aromatic ring radical that comprises two to twelve carbon atoms and from one to six heteroatoms selected from nitrogen, oxygen and sulfur. Unless stated otherwise specifically in the specification, the heterocycloalkyl radical is a monocyclic, bicyclic, tricyclic or tetracyclic ring system, which may include fused or bridged ring systems. The heteroatoms in the heterocycloalkyl radical may be optionally oxidized. One or more nitrogen atoms, if present, are optionally quaternized. The heterocycloalkyl radical is partially or fully saturated. The heterocycloalkyl may be attached to the rest of the molecule through any atom of the ring(s). Examples of such heterocycloalkyl radicals include, but are not limited to, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl. Unless stated otherwise specifically in the specification, a heterocycloalkyl moiety is optionally substituted by one or more substituents which are described herein as suitable substitution groups.

In some embodiments, the definition of terms used herein is according to IUPAC.

### EXAMPLES

### Example 1: Results of Computational M. smithii Enzyme-Ligand Docking Experiments

Protein-ligand docking experiments were conducted to investigate whether, in addition to their cholesterol-lowering effects, HMG-CoA reductase inhibitors (specifically lovastatin lactone) directly inhibit enzymes in the archaeal methanogenesis pathway. Methanobrevibacter smithii F420-dependent methylenetetrahydromethanopterin dehydrogenase (mtd), a key methanogenesis enzyme with a known sequence but no tertiary protein structural information, was modeled using Protein Databank (PDB) templates employing Eidogen STRUCTFAST technology (Bioinformatics. 2005 Jun. 15; 21(12):2827-31).

Ligand binding sites were identified by inference from the respective PDB templates used in modeling and from the Eidogen SiteSeeker algorithm, were used to develop models for A5UMI1 and Q02394.

Other sites were manually inferred within PyMOL v1.8 after aligning models and templates containing their respective co-complexed ligands. Residues on model structures with a 7 Å cutoff of co-complexed ligands within the templates were exported and also processed as sites.

The ligands for docking were the F420-coenzyme (as natural ligand), lovastatin and simvastatin, both in their respective lactone and β-hydroxyacid forms.

Ligands were carefully prepared considering different protonation states, isomers, and tautomers. Charges were standardized, missing hydrogens added, ionization states enumerated, functional groups ionized, tautomers and isomers generated, and starting-point 3D coordinates for each ligand using BIOVIA's (Accelrys') Pipeline Pilot technology v8.5 generated. Ligands were finally prepared into mol2 format and then docked into each identified site and scored using AutoDock Vina v1.1.2.

A total of 88 ligand variations were systematically docked across the extracted 12 sites for a total of 1,056 docking simulations. Because the docking process scores ligand conformations based on ligand conformation and ligand-to-receptor interactions within a grid box, after the 1,056 docking simulations were complete, all docked ligand variations against their respective full model structures were rescored.

Three different PDB templates that had sufficient sequence homology to model the Q02394 sequence were identified. The top three PDBs showing significant sequence homology to Q02394 included: 3F47 (57%), 3H65 (57%), and 4JJF (52%). Each template was used to model Q02394.

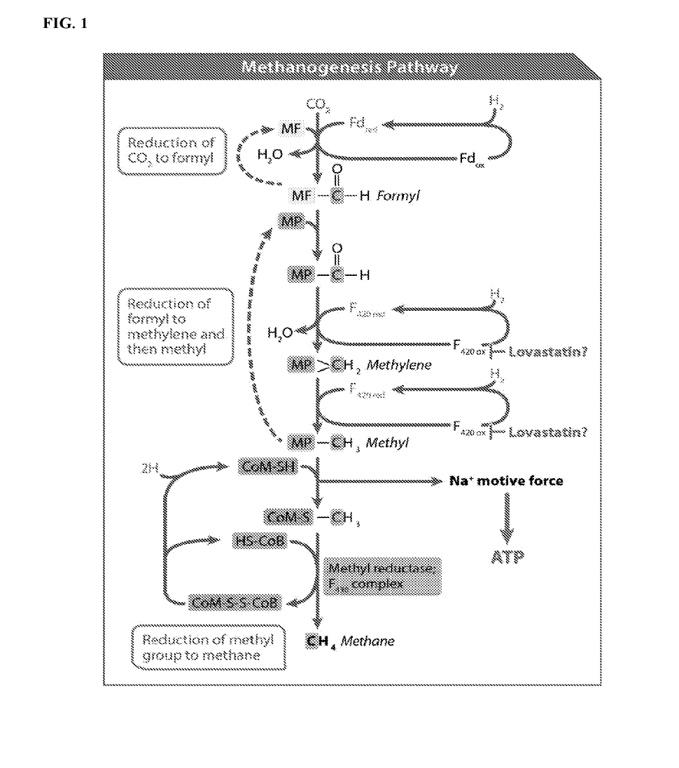

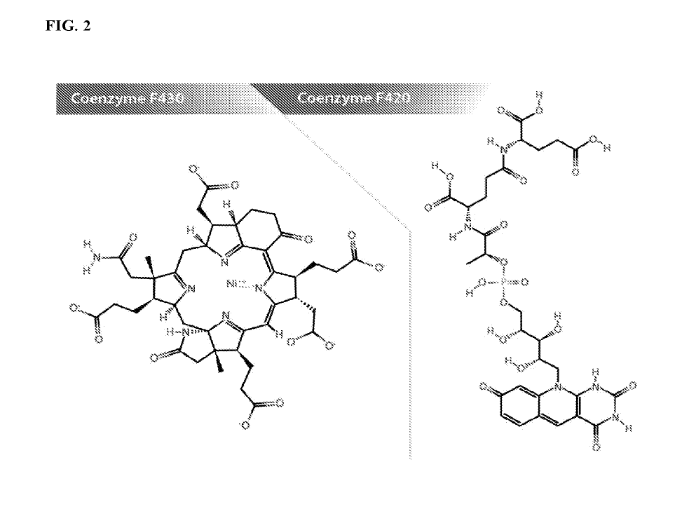

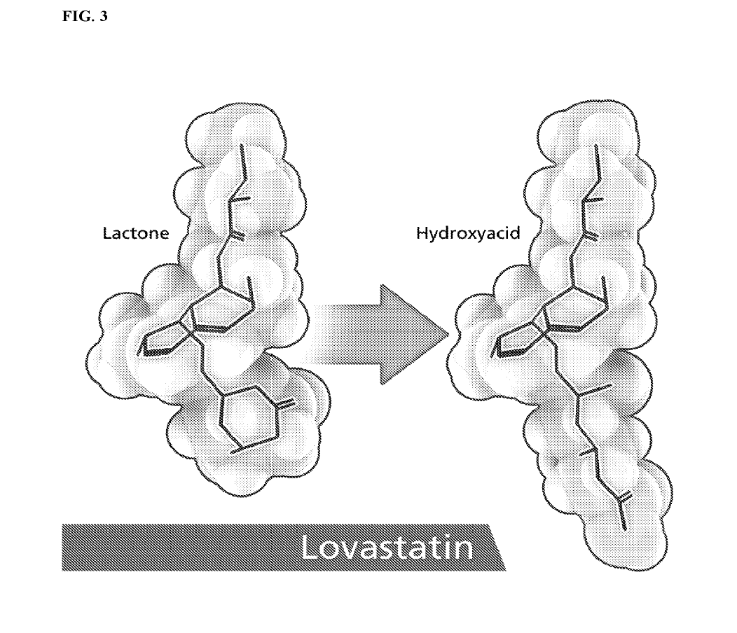

The modeling of sequence A5UMI1 was straightforward given its high 52% sequence homology to 3IQZ. The Eidogen SiteSeeker algorithm identified only one site when template chains A, C, D were used, while two sites were identified in models leveraging template chains B, E, F. The H4M site was modeled manually (FIG. 1).

Four ligand sites from the A5UMI1 modeling and six sites from Q02394 modeling were used in the docking simulations.

Key ligands included lovastatin (lactone and hydroxyacid forms), F420, and simvastatin (lactone and Hydroyacid forms).

Process ligands found in PB3 templates were used to model sequences and computationally processed prior to docking.

A total of 88 ligand variations were docked into 10 identified binding sites across all models for a total of 880 docking simulations.

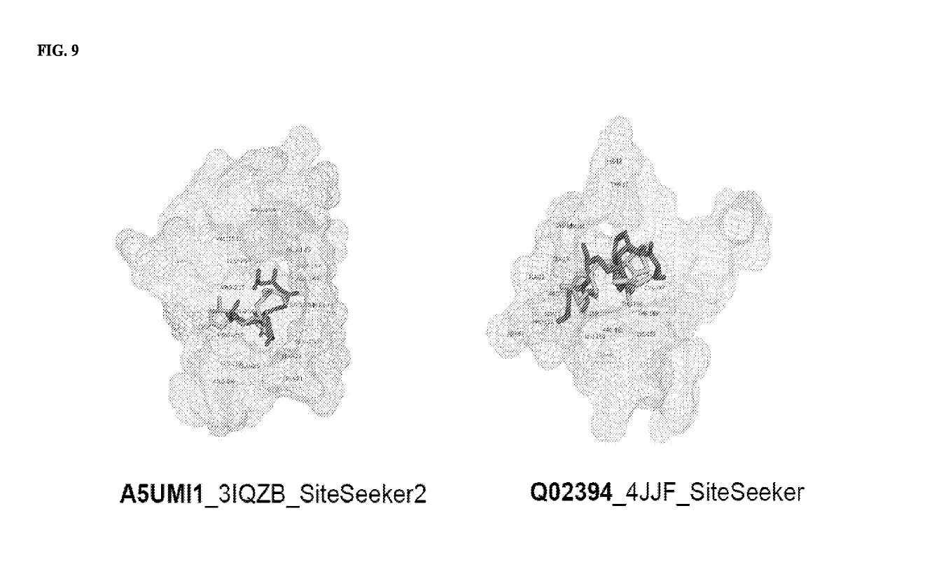

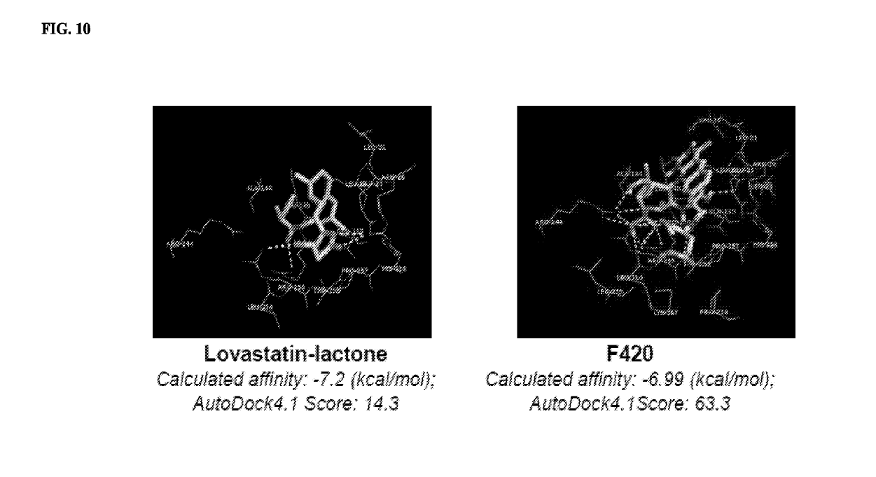

The top two scoring sites were A5UMI1_3IQZB and Q02394_4JJF. These were used to rank order each ligand (FIG. 9).

The lactone form statins docked into each site with favorable site interactions (i.e. lower docking scores) as compared to F420 for the same sequence/site grouping.

The statin lactone forms generally had more favorable docking scores, even relative to the native template PDB ligands.

The statin acid forms had less favorable docking scores and typically scored in the middle with some of the F420 forms.

The F420 scores were generally the lowest for each sequence/site models of A5UM1 and Q02394.

The conclusions of this Examples are, in part, statin binding is likely for the two key targets: A5UMI1 and Q02394; lactone forms of statins exhibit preferential binding over the native-F420 coenzyme ligand in silico and thus could inhibit the activity of the key M. smithii methanogenesis enzyme mtd in vivo; and statin lactones may exert a methane-reducing effect which is distinct from their cholesterol lowering activity.

### Example 2: Identification of F420-Modulating Compounds

A high-throughput screening was utilized to identify compounds that bind to and modulate the activities of F₄₂₀-dependent enzymes. Specifically, screening was conducted to identify compounds that exhibit improved binding to F₄₂₀-dependent enzymes as compared to statins (e.g., lovastatin) using a Pharmacophoric Fingerprinting (PFP) methodology, as described, for example, in McGregor et al. (1999) J. Chem. Inf. Comput. Sci. 39:569-574 and McGregor et al. (2000) J. Chem. Inf. Comput Sci. 40:117-125, the entire contents of which are hereby incorporated by reference.

Generally, lovastatin served as a pharmacophore against which a library of 9 million compounds was screened. The top hits were then docked to four different proteins (e.g., three well-characterized Mycobacterium tuberculosis (Mib) F₄₂₀ enzymes and one Pseudomonas control). The top hits were then collected and divided into five scaffold groups. A list of thirty lead candidates that bind significant better to the targets than lovastatin was selected for further studies. The following compounds are identified through the screen:

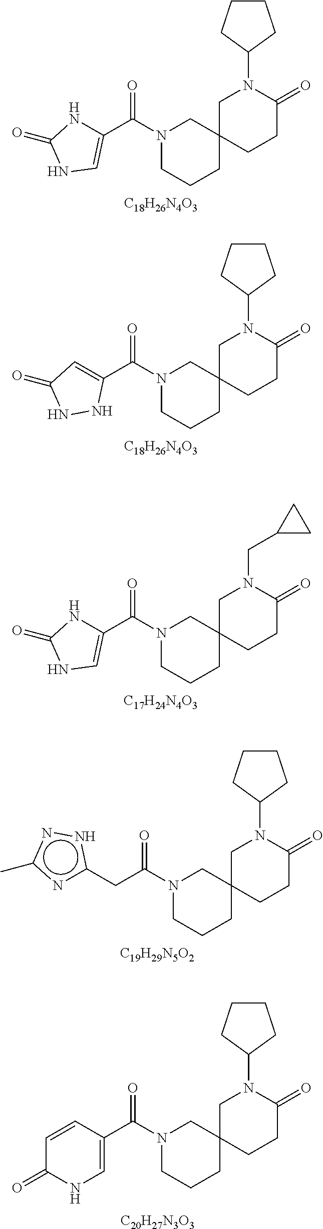

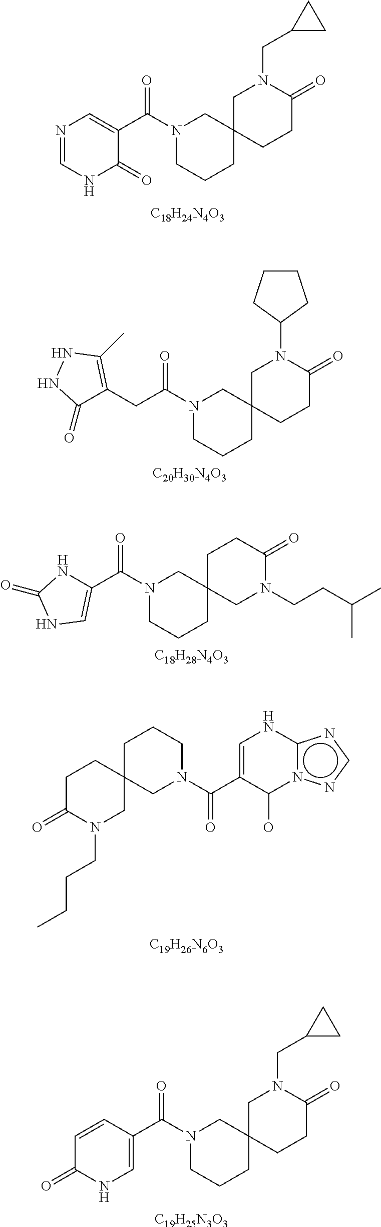

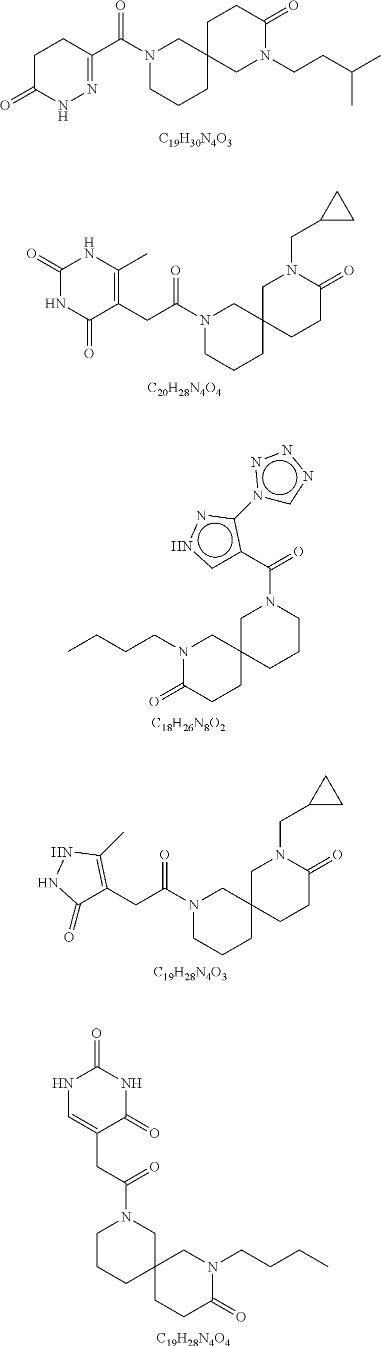

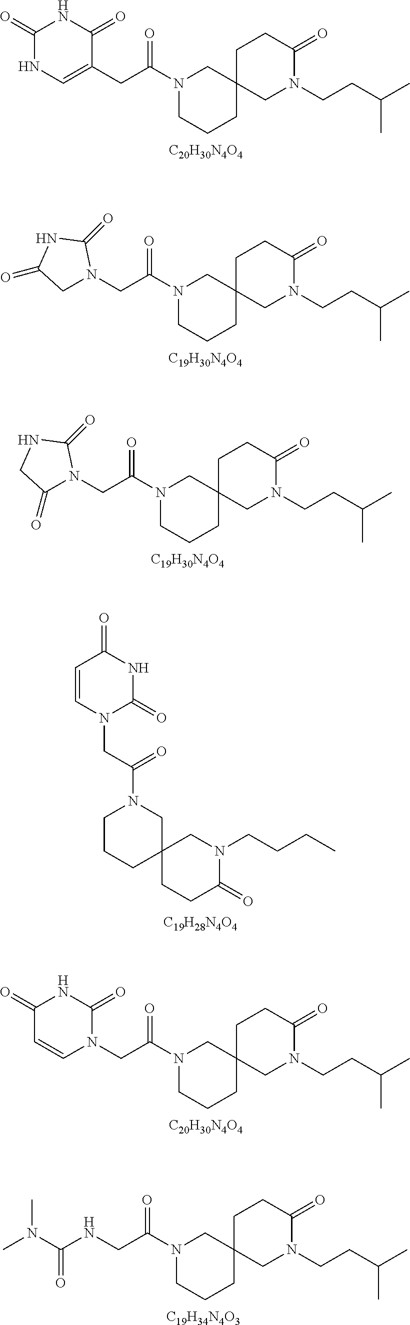

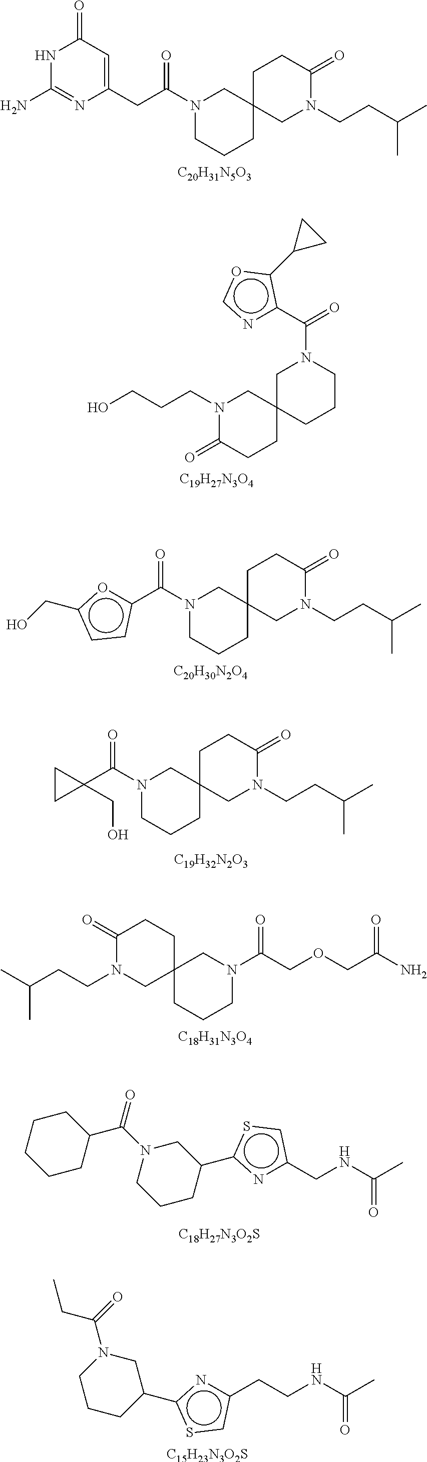

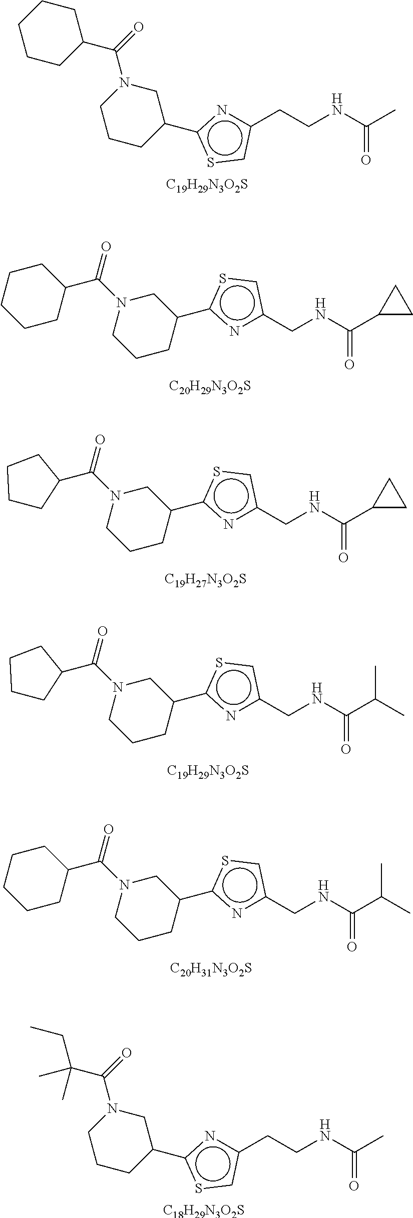

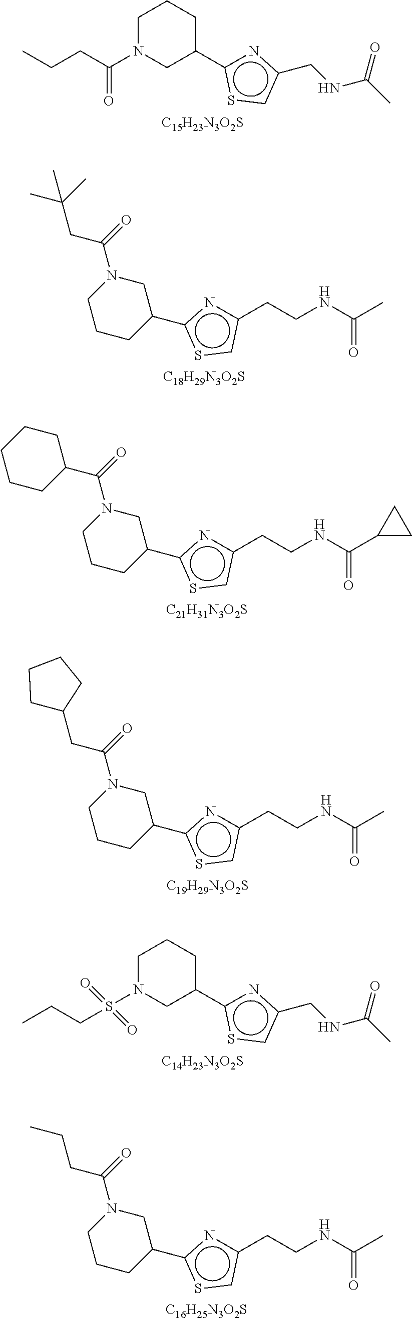

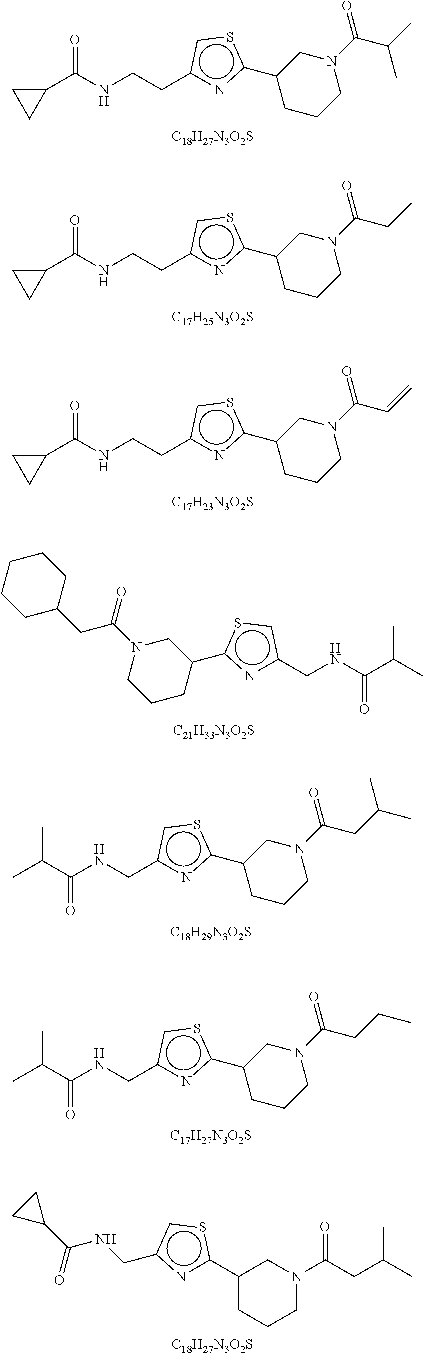

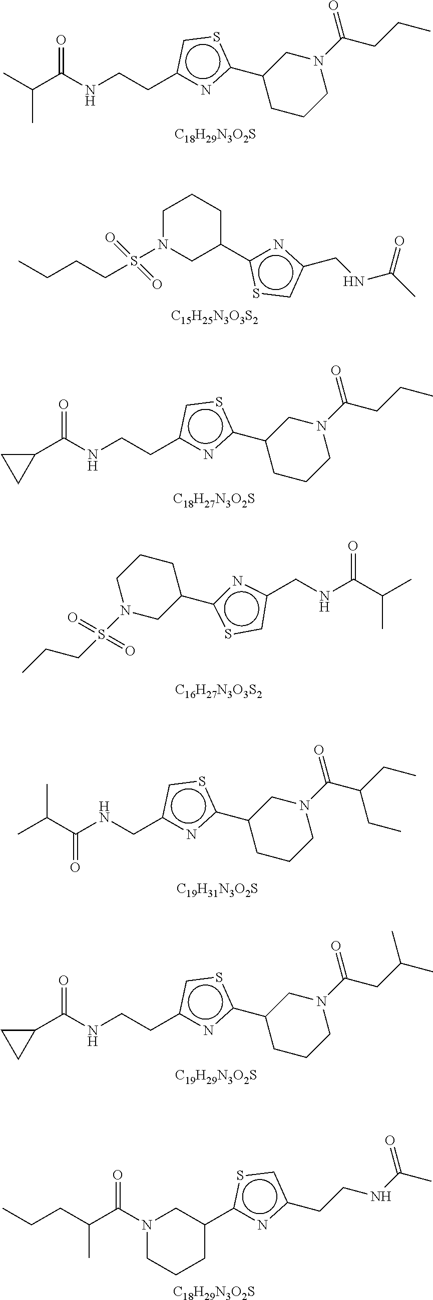

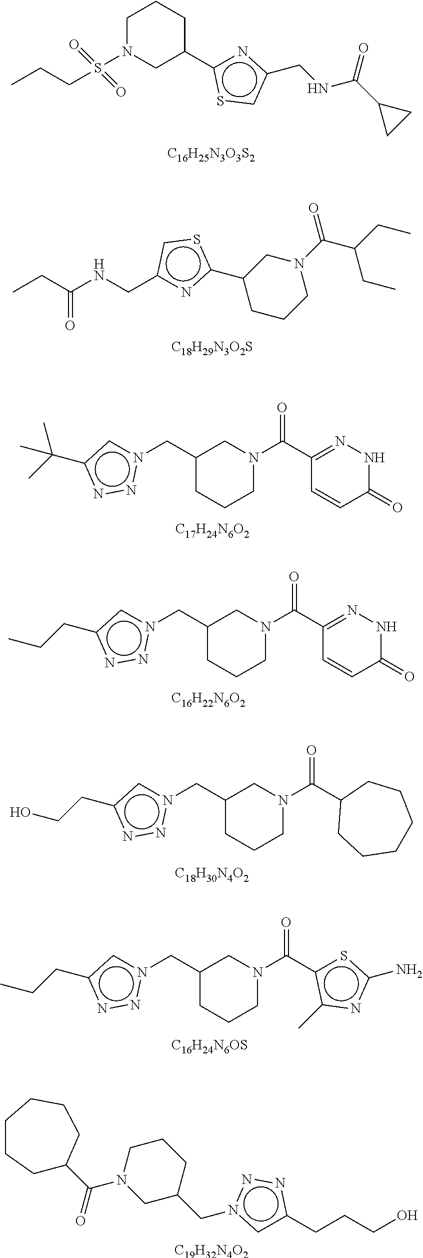

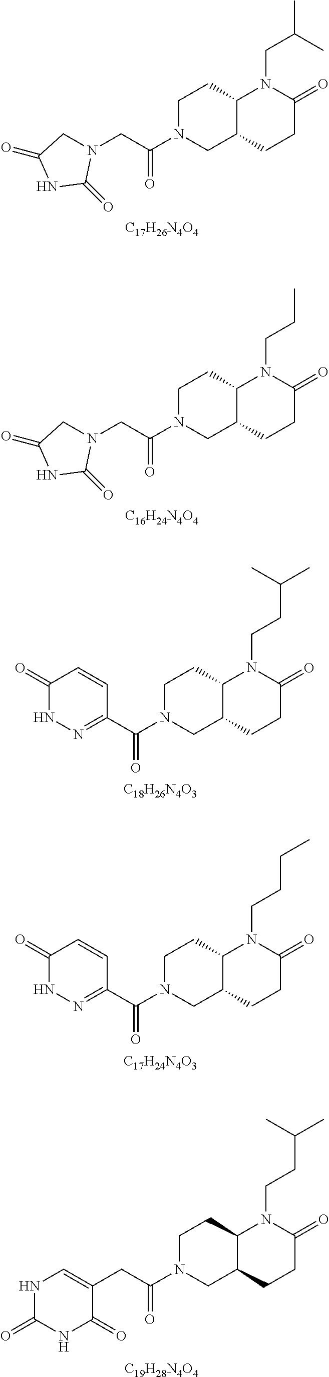

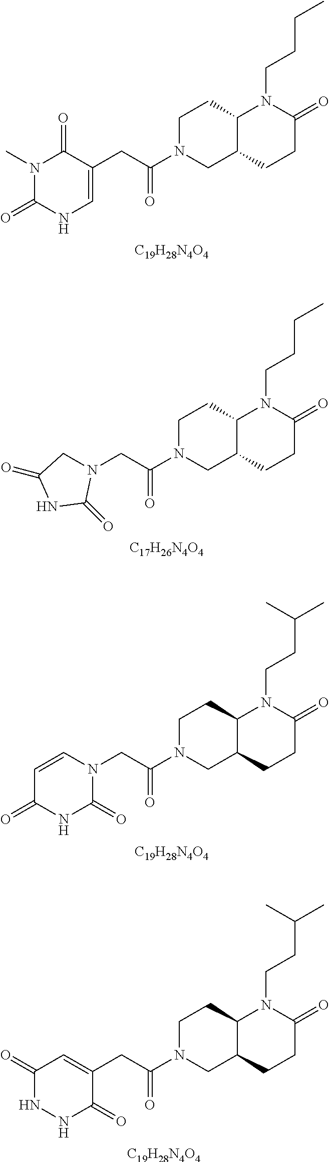

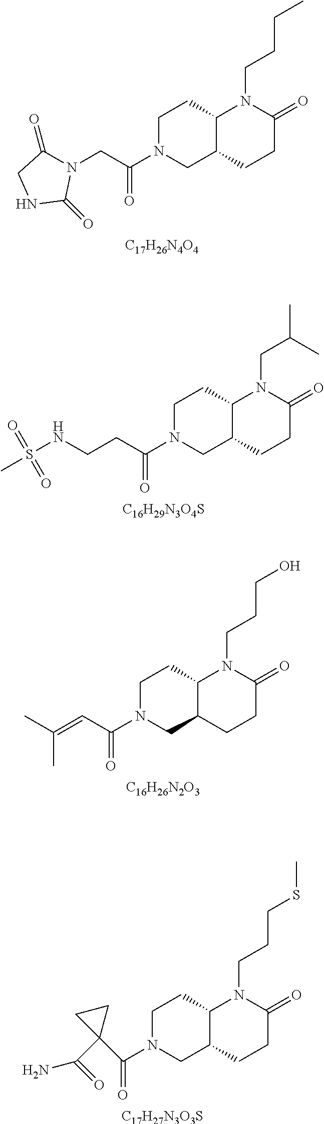

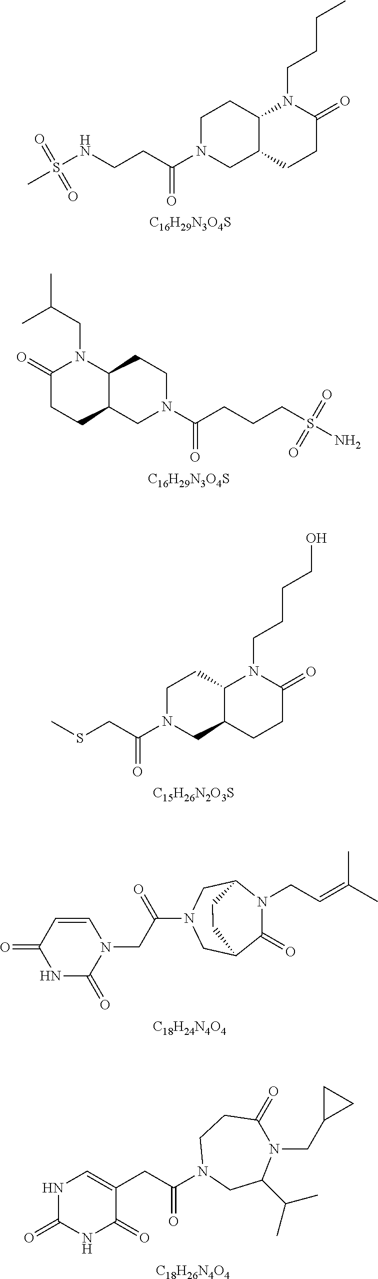

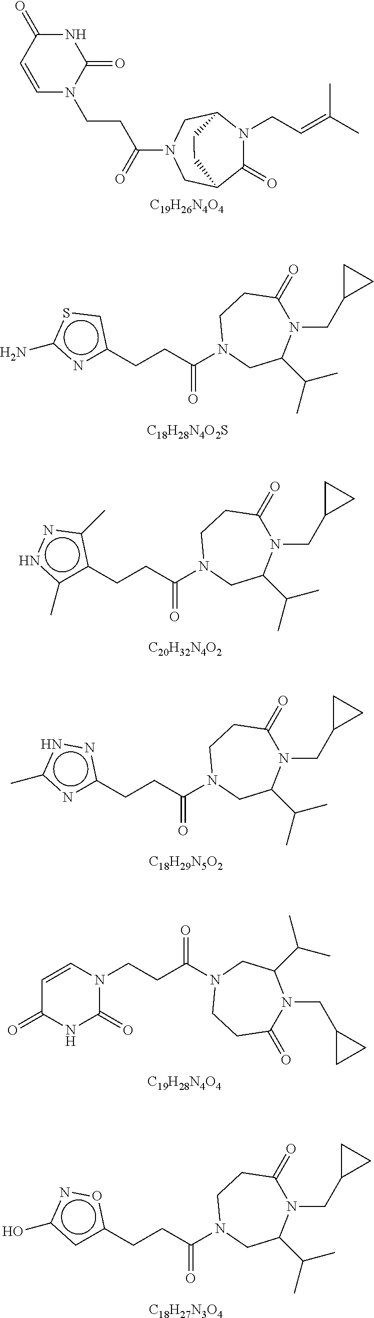

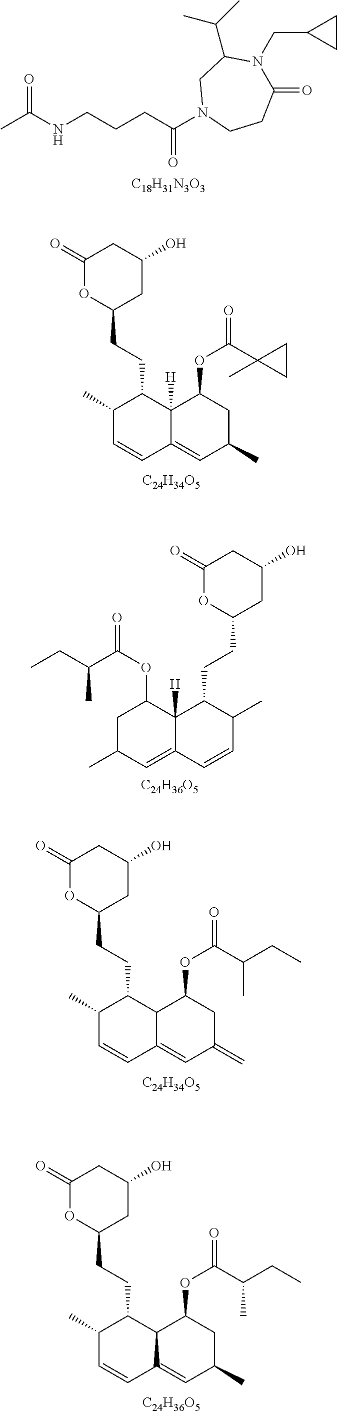

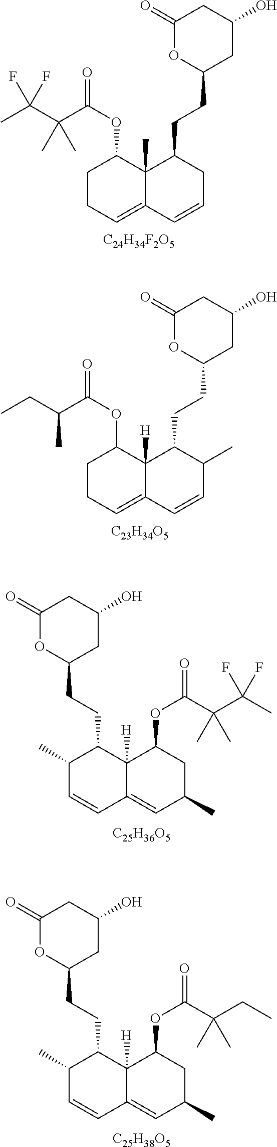

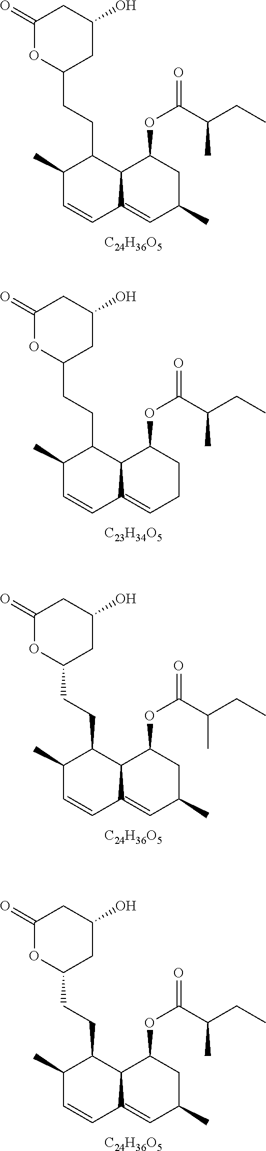

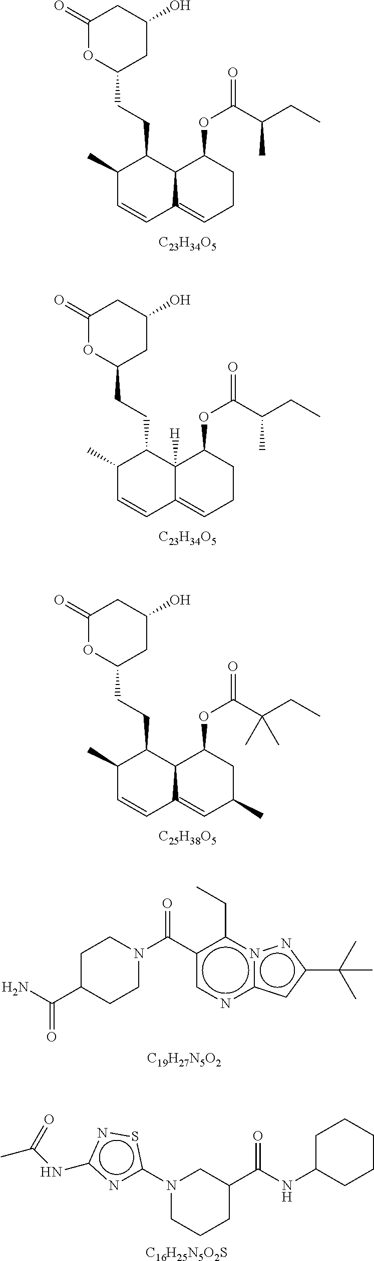

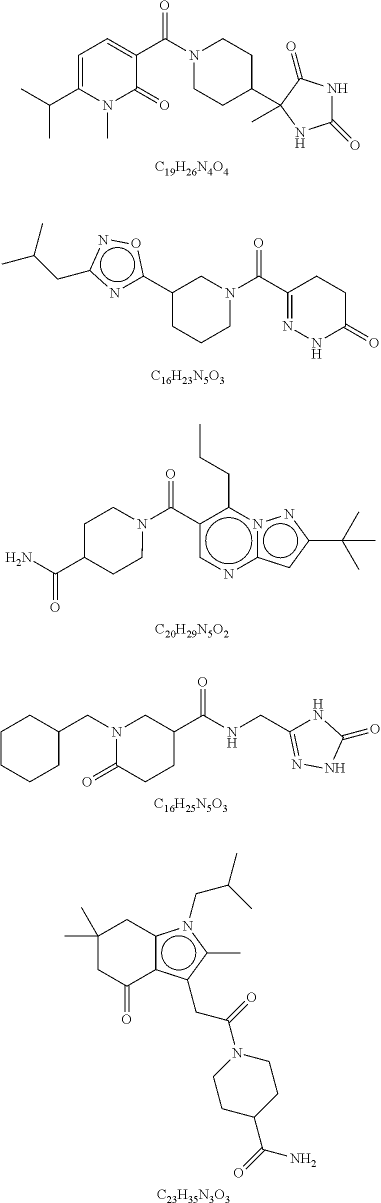

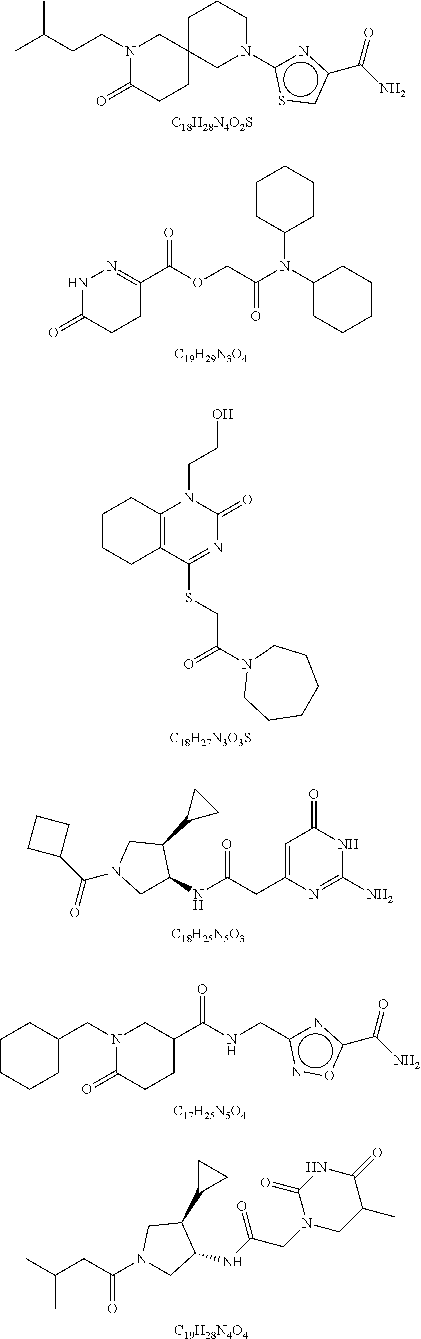

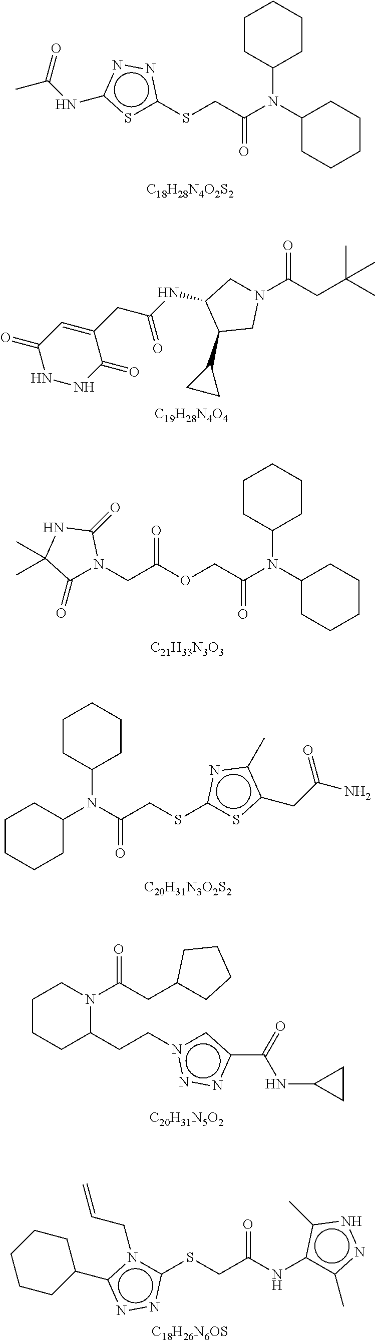

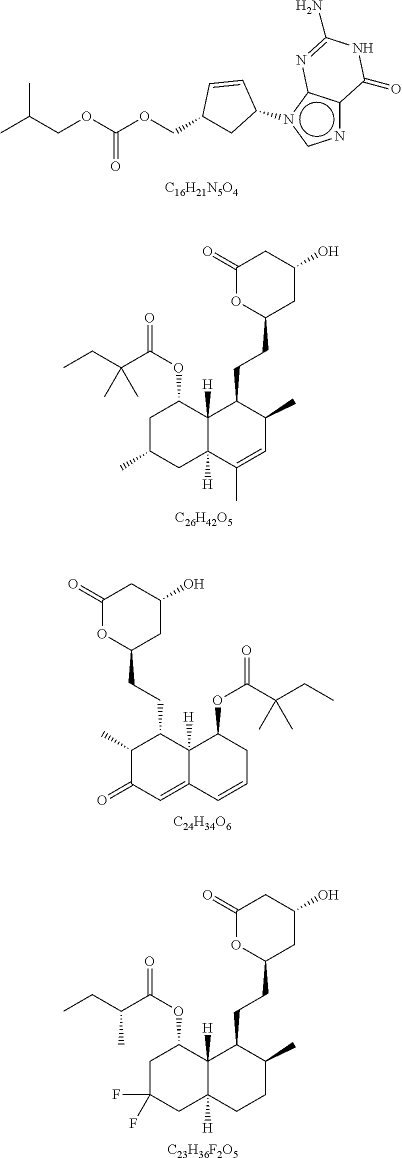

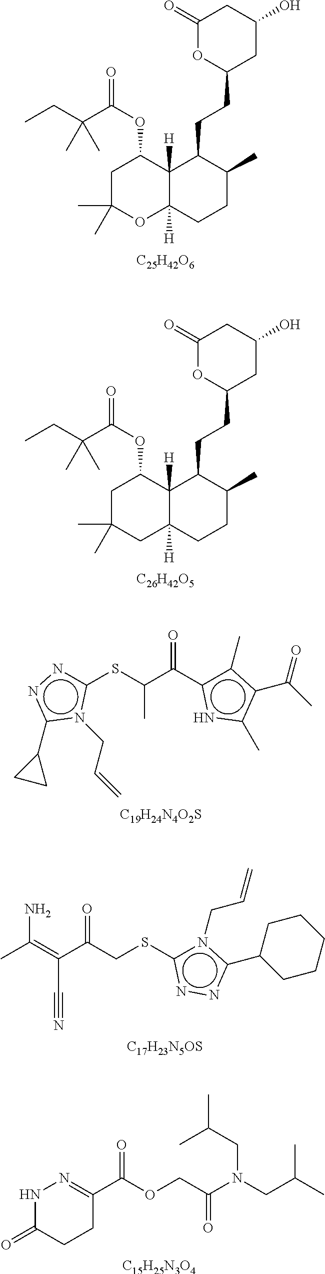

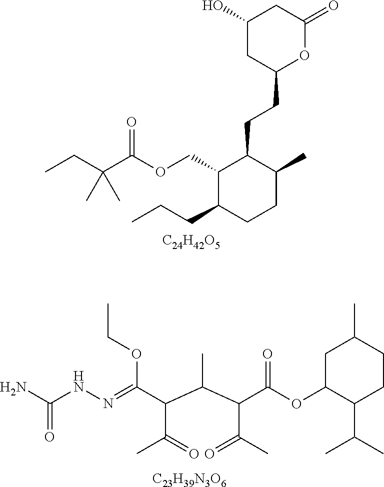

### Example 3: Inhibition of Methanogenesis

Experiments are carried out to test the effectiveness of the various compounds identified in Example 1 for inhibiting methanogenesis in various methanogens. MIC be determined.

For instance, Methanobrevibacter smithii will be evaluated.

The various methanogens tested include Methanobrevibacter smithii, Methanobrevibacter ruminantium, Methanobacterium bryantii, Methanobacterium formicum, Methanobrevibacter arboriphilicus, Methanobrevibacter gottschalkii, Methanocalculus chunghsingensis, Methanococcoides burtonii, Methanococcus aeolicus, Methanococcus deltae, Methanococcus jannaschii, Methanococcus maripaludis, Methanococcus vannielii, Methanocorpusculum labreanum, Methanoculleus bourgensis (Methanogenium olentangyi, Methanogenium bourgense), Methanoculleus marisnigri, Methanofollis liminatans, Methanogenium cariaci, Methanogenium frigidum, Methanogenium organophilum, Methanogenium wolfei, Methanomicrobium mobile, Methanopyrus kandleri, Methanoregula boonei, Methanosaeta concilii, Methanosaeta thermophila, Methanosarcina acetivorans, Methanosarcina barkeri, Methanosarcina mazei, Methanosphaera stadtmanae, Methanospirillium hungatei, Methanothermobacter defluvii (Methanobacterium defluvii), Methanothermobacter thermautotrophicus (Methanobacterium thermoautotrophicum), Methanothermobacter thermoflexus (Methanobacterium thermoflexum), Methanothermobacter wolfei (Methanobacterium wolfei), and Methanothrix sochngenii. As a control, the Pseudomonas aeruginosa and Gentamicin methicillin resistant staphylococcus (GMRSA) are also tested.

Results indicate that the compounds effectively inhibit methanogenesis in the various methanogens tested.

### EQUIVALENTS

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth and as follows in the scope of the appended claims.

Those skilled in the art will recognize, or be able to ascertain, using no more than routine experimentation, numerous equivalents to the specific embodiments described specifically herein. Such equivalents are intended to be encompassed in the scope of the following claims.

### INCORPORATION BY REFERENCE

All patents and publications referenced herein are hereby incorporated by reference in their entireties.

The publications discussed herein are provided solely for their disclosure prior to the filing date of the present application. Nothing herein is to be construed as an admission that the present invention is not entitled to antedate such publication by virtue of prior invention.

As used herein, all headings are simply for organization and are not intended to limit the disclosure in any manner. The content of any individual section may be equally applicable to all sections.

What is claimed is:

1. A method of modulating a methanogenesis-related F₄₂₀-dependent enzyme, comprising administering an effective amount of a statin, a statin analog or derivative, or a compound disclosed herein to a subject in need thereof.

2. The method of claim 1, wherein the F₄₂₀-dependent enzyme is an enzyme in the methanogenesis pathway.

3. The method of claim 2, wherein the F₄₂₀-dependent enzyme is selected from mtd/A5UMI1 (F₄₂₀-dependent methylenetetrahydromethanopterin dehydrogenase), fno F₄₂₀-dependent NADP oxidoreductase, mtd F₄₂₀-dependent methylene-H4MPT dehydrogenase, mer F₄₂₀-dependent methylene-H4MPT reductase, coenzyme F₄₂₀ hydrogenase, and F₄₂₀-dependent sulfite reductase.

4. The method of claim 3, wherein the F₄₂₀-dependent enzyme is mtd/A5UMI1 (F₄₂₀-dependent methylenetetrahydromethanopterin dehydrogenase).

5. A method of treating a gastrointestinal (GI) disorder, comprising administering an effective amount of a compound disclosed herein to a subject in need thereof.

6. The method of claim 5, wherein the gastrointestinal (GI) disorder is irritable bowel syndrome, optionally constipation-associated IBS (IBS-C).

7. A method of treating obesity, comprising administering an effective amount of a compound disclosed herein to a subject in need thereof.

8. A method of treating diabetes, comprising administering an effective amount of a compound disclosed herein to a subject in need thereof.

9. The method of any one of the above claims, wherein the subject is human.

10. The method of any one of claims 1-9, wherein the compound has the structure of Formula I: 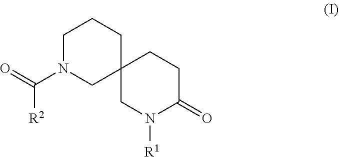
or pharmaceutically acceptable salts, stereoisomers, or prodrugs derivatives thereof,
wherein:
R¹ is selected from C₁-C₆ alkyl, hydroxy-C₁-C₆ alkyl, (C₃-C₆ cycloalkyl)-C₁-C₃ alkyl, and C₃-C₆ cycloalkyl;
R² is selected from R^{hc}, —CH₂—R^{hc}, —CH₂CH₂—R^{hc}, C₃-C₆ cycloalkyl (optionally substituted with C₁-C₃ alkyl or hydroxy-C₁-C₃ alkyl), heteroalkyl (optionally substituted with one or more moieties independently selected from oxo, amino (—NH₂), (C₁-C₃ alkyl)amino, and di(C₁-C₃ alkyl)amino); and
R^{hc} is selected from 5- or 6-membered heterocyclic ring (optionally substituted with one or more moieties independently selected from oxo, tetrazolyl, C₃-C₆ cycloalkyl, hydroxy-C₁-C₃ alkyl, C₁-C₃ alkyl, and amino (—NH₂)), heterocyclic ring system (optionally substituted with oxo, amino (—NH₂), (C₁-C₃ alkyl)amino, and di(C₁-C₃ alkyl)amino)).

11. The method of claim 10, wherein the compound is one of Compounds (1)-(26), or pharmaceutically acceptable salts, stereoisomers, or prodrug derivatives thereof.

12. The method of any one of claims 1-9, wherein the compound has the structure of Formula IIa-IIc: 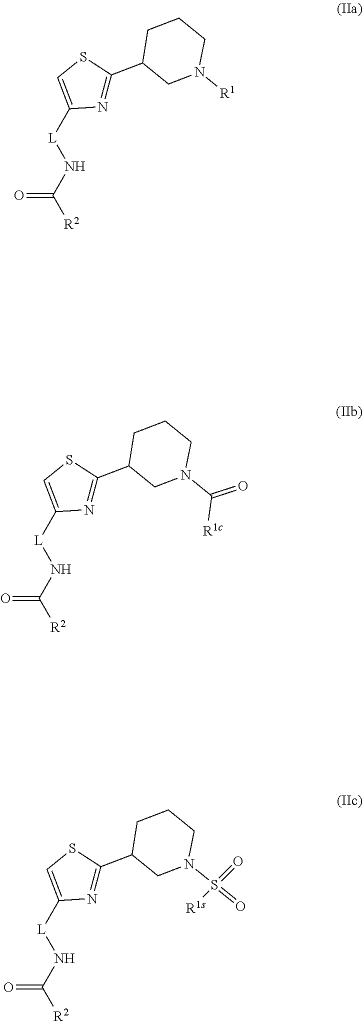
or pharmaceutically acceptable salts, stereoisomers, or prodrugs derivatives thereof, wherein:
L is a bond selected from —CH₂— and —CH₂CH₂—;
R¹ is selected from —C(O)R^{1c} and —SO₂R^{1s};
R^{1c} is selected from C₁-C₆ alkyl, C₃-C₇ cycloalkyl-C₁-C₃ alkyl, C₂-C₆ alkenyl, and C₃-C₇ cycloalkyl;
R^{1s} is C₁-C₆ alkyl, C₃-C₇ cycloalkyl-C₁-C₃ alkyl, C₂-C₆ alkenyl, and C₃-C₇ cycloalkyl; and
R² is selected from C₁-C₆ alkyl and C₃-C₆ cycloalkyl.

13. The method of claim 12, wherein the compound is one of Compounds (27)-(56), or pharmaceutically acceptable salts, stereoisomers, or prodrugs derivatives thereof.

14. The method of any one of claims 1-9, wherein the compound has the structure of Formula III: 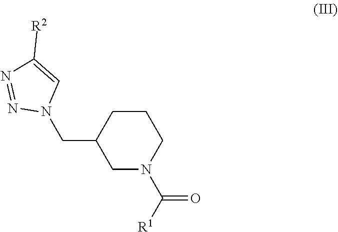
or pharmaceutically acceptable salts, stereoisomers, or prodrugs derivatives thereof, wherein:
R¹ is selected from C₅-C₈ cycloalkyl and 5- or 6-membered heterocyclic ring (optionally substituted with one or more moieties independently selected from oxo, C₁-C₃ alkyl, and amino (—NH₂)); and
R² is selected from C₁-C₆ alkyl and hydroxy-C₁-C₆ alkyl.

15. The method of claim 14, wherein the compound is one of Compounds (57)-(61), or pharmaceutically acceptable salts, stereoisomers, or prodrugs derivatives thereof.

16. The method of any one of claims 1-9, wherein the compound has the structure of Formula IV: 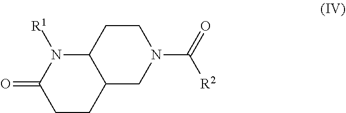
or pharmaceutically acceptable salts, stereoisomers, or prodrugs derivatives thereof, wherein:
R¹ is selected from C₁-C₆ alkyl, hydroxy-C₁-C₆ alkyl, and (C₁-C₃ alkyl)thio-C₁-C₆ alkyl;
R² is selected from R^{hc}, —CH₂—R^{hc}, —CH₂CH₂—R^{hc}, C₃-C₆ cycloalkyl (optionally substituted with carbamoyl (—C(O)NH₂) or N—(C₁-C₃ alkyl)-carbamoyl), C₂-C₆ alkenyl, C₁-C₆ alkyl (optionally substituted with (C₁-C₃ alkyl)sulfonamido (—NHSO₂(C₁-C₃ alkyl)), sulfamoyl (—SO₂NH₂), or N—(C₁-C₃ alkyl)-sulfamoyl), and (C₁-C₃ alkyl)thio-C₁-C₆ alkyl; and
R^{hc} is a 5- or 6-membered heterocyclic ring (optionally substituted with one or more moieties independently selected from oxo and C₁-C₃ alkyl).

17. The method of claim 16, wherein the compound is one of Compounds (62)-(77), or pharmaceutically acceptable salts, stereoisomers, or prodrugs derivatives thereof.

18. The method of any one of claims 1-9, wherein the compound has the structure of Formula V: 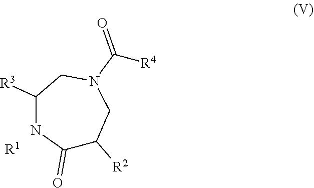
or pharmaceutically acceptable salts, stereoisomers, or prodrugs derivatives thereof, wherein:
R¹ is selected from (C₃-C₆ cycloalkyl)-C₁-C₃ alkyl and C₂-C₆ alkenyl;
R² is H and R³ is C₁-C₆ alkyl; or R² and R³ are joined to form a C₁-C₆ alkyl bridge;
R⁴ is selected from —R^{hc}, —CH₂—R^{hc}, and —CH₂CH₂—R^{hc}; and
R^{hc} is a 5- or 6-membered heterocyclic ring (optionally substituted with one or more moieties independently selected from oxo, amino (—NH₂), C₁-C₃ alkyl, and hydroxy).

19. The method of claim 18, wherein the compound is one of Compounds (78)-(86), or pharmaceutically acceptable salts, stereoisomers, or prodrugs derivatives thereof.

20. The method of any one of claims 1-9, wherein the compound has the structure of Formula VI: 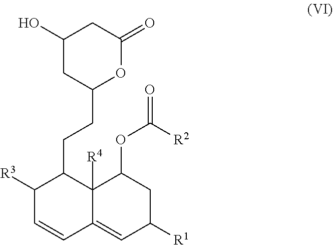
or pharmaceutically acceptable salts, stereoisomers, or prodrugs derivatives thereof, wherein:
R¹ is selected from H, C₁-C₃ alkyl, =CH₂, =CHCH₃, =C(CH₃)₂, and =CHCH₂CH₃;
R² is selected from C₃-C₆ cycloalkyl (optionally substituted with C₁-C₃ alkyl), C₁-C₆ alkyl, and halo-C₁-C₆ alkyl;
R³ is selected from H and C₁-C₃ alkyl; and
R⁴ is selected from H, and C₁-C₃ alkyl.

21. The method of claim 20, wherein the compound is one of compounds (87)-(101), or pharmaceutically acceptable salts, stereoisomers, or prodrugs derivatives thereof.

22. The method of any one of claims 1-9, wherein the compound is one of compounds (102)-(131), or pharmaceutically acceptable salts, stereoisomers, or prodrugs derivatives thereof.

23. A method of selecting a subject for treatment with a statin, a statin analog or derivative, or a compound disclosed herein, comprising:
(i) obtaining a biological sample from the subject;
(ii) profiling the status of an enzyme in the methanogenesis pathway in the biological sample; and
(ii) selecting the subject for treatment with the statin, statin analog or derivative, or compound disclosed herein if the enzyme in the methanogenesis pathway is present at high levels in the biological sample.

24. The method of claim 23, wherein the biological sample is selected from stool, mucosal biopsy from a site in the gastrointestinal tract, aspirated liquid from a site in the gastrointestinal tract, or combinations thereof.

25. The method of claim 23 or 24, wherein the enzyme in the methanogenesis pathway is mtd/A5UMI1.

26. A method of making an agent effective for the treatment of a methanogen-related disorder, comprising:
(a) identifying the agent by:
(i) contacting a test agent with an enzyme in the methanogenesis pathway;
(ii) determining a binding affinity and/or inhibition activity of the test agent with an enzyme in the methanogenesis pathway; and
(iii) selecting the test agent as a candidate agent if the result of the comparison of step (ii) indicates that the test agent is useful for the methanogen-related disorder; and
(b) formulating the candidate agent for administration to the GI tract.

27. The method of claim 26, wherein the methanogen-related disorder is irritable bowel syndrome.

28. The method of claim 26, wherein the methanogen-related disorder is irritable bowel syndrome with constipation (IBS-C).

29. The method of claim 26, wherein the methanogen-related disorder is obesity.

30. The method of claim 26, wherein the methanogen-related disorder is diabetes.

31. The method of any one of claims 26-30, wherein the enzyme in the methanogenesis pathway is selected from mid/A5UMI1 (F₄₂₀-dependent methylenetetrahydromethanopterin dehydrogenase), fno F₄₂₀-dependent NADP oxidoreductase, mtd F₄₂₀-dependent methylene-H4MPT dehydrogenase, mer F₄₂₀-dependent methylene-H4MPT reductase, coenzyme F₄₂₀ hydrogenase, and F₄₂₀-dependent sulfite reductase.

32. The method of claim 31, wherein the enzyme in the methanogenesis pathway is mtd/A5UMI1.

33. The method of claim 31 or claim 32, wherein the identifying step uses a biological sample from a methanogen-related disorder patient.

34. The method of claim 33, wherein the biological sample is selected from stool, mucosal biopsy from a site in the gastrointestinal tract, aspirated liquid from a site in the gastrointestinal tract, or combinations thereof.

35. A pharmaceutical composition comprising one or more of Compounds (1)-(131) and a pharmaceutically acceptable excipient.
